(19) United States
(12) Patent Application Publication
(10) Pub. No.: US 2025/0215011 A1
(43) Pub. Date: Jul. 3, 2025

(54) T-TYPE CALCIUM CHANNEL MODULATORS COMPRISING A DIAZASPIROHEPTANE CORE AND METHODS OF USE THEREOF

(71) Applicant: Praxis Precision Medicines, Inc., Boston, MA (US)

(72) Inventors: Ricardo Lira, East Lyme, CT (US); Andrew Mark Griffin, Montreal (CA); Jon Sutton, Essex (GB); Lampros Milanos, Saffron Walden (GB); Simon Crumpler, Harlow (GB)

(21) Appl. No.: 18/852,396

(22) PCT Filed: Apr. 3, 2023
(86) PCT No.: PCT/US2023/017268

§ 371 Date: Sep. 27, 2024

Related U.S. Application Data
(60) Provisional application No. 63/326,699, filed on Apr. 1, 2022.

(51) Int. Cl. C07D 487/10; A61K 31/397; A61K 31/4184

(52) U.S. Cl. CPC C07D 487/10; A61K 31/397; A61K 31/4184

(57) ABSTRACT

Disclosed herein are compounds for treating a condition modulated by calcium channel ion activity and pharmaceutical compositions comprising the compounds, wherein the compounds comprise a diazaspiroheptane core and left- and right-hand substitutions of the diazaspiroheptane core. Also disclosed herein are methods using the compounds to treat a disease or condition relating to aberrant function or activity of a T-type calcium channel.

### RELATED APPLICATIONS

This application claims the benefit of priority to U.S. Provisional Application No. 63/326,699, filed on Apr. 1, 2022, the entire contents of which are incorporated herein by reference.

### FIELD OF THE DISCLOSURE

Disclosed herein are compounds for treating conditions associated with calcium channel activity and in particular T-type calcium channel activity. Specifically, disclosed herein are compounds comprising a diazaspiroheptane core, wherein the compounds comprise left and right-hand substitutions. Also disclosed herein are methods of treating conditions associated with T-type calcium channel activity by administering the compounds disclosed herein.

### BACKGROUND OF THE DISCLOSURE

The entry of calcium into cells through voltage-gated calcium channels mediates a wide variety of cellular and physiological responses, including excitation-contraction coupling, hormone secretion and gene expression. In neurons, calcium channels directly affect membrane potential and contribute to electrical properties, as well as modulating the activity of calcium-dependent enzymes such as protein kinases C and calmodulin-dependent protein kinase II. T-type calcium channels are low-voltage activated ion channels that mediate the influx of calcium into cells.

T-type calcium channel modulators having a piperidinyl core are disclosed, for example, in PCT Publication No. 2009/146540, published 10 Dec. 2009, and U.S. Pat. Nos. 8,377,968 (published 19 Feb. 2013), 8,569,344 (published 29 Oct. 2013), and 9,096,522 (published 4 Aug. 2015), the entire contents of which are incorporated by reference herein.

Aberrant function of these T-type calcium ion channels is associated with several diseases or conditions, including psychiatric disorders (e.g., mood disorders such as major depressive disorder), pain, tremor (e.g., essential tremor), epilepsy, or an epilepsy syndrome (e.g., absence seizures and juvenile myoclonic epilepsy). Accordingly, additional compounds that selectively modulate T-type calcium channels in mammals may be useful in treatment of such disease states.

### SUMMARY OF THE DISCLOSURE

The present disclosure provides compounds for treating conditions associated with calcium channel activity and in particular T-type calcium channel activity. Specifically, disclosed herein are compounds of Formula (I) or Formula (IA) comprising a diazaspiroheptane core comprising right and left-hand substitutions.

In one aspect, the compounds disclosed herein comprise a compound of Formula (IA) with a diazaspiroheptane core:

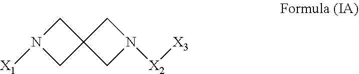

wherein X₁ is a left-hand substitution of the diazaspiroheptane core chosen from:

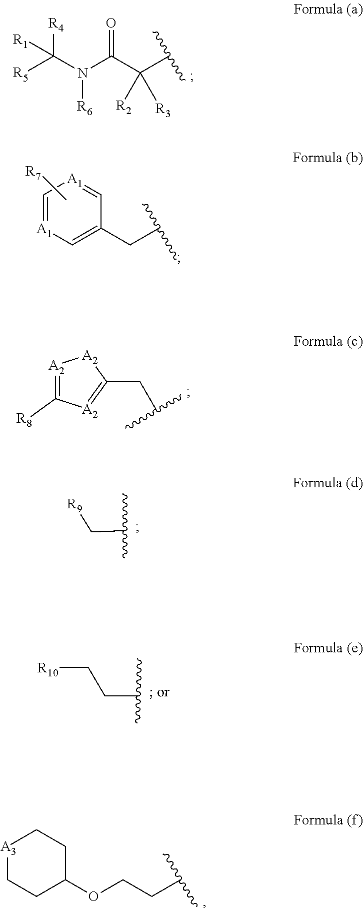

wherein R₁ is chosen from —H, —CH₃, —CH₂OCH₃, —CF₃, —CH₂CH₃, or —(CH₂)₂OCH₃;
R₂ is —H;
R₃ is —H;
R₄ is chosen from —H or —CH₃, or R₁ and R₄ together form a cyclopropane, a cyclobutane, a cyclopentane, or an oxetane ring;
R₅ is chosen from —H, —CH₃, —CF₃, —CH₂OH, —COOCH₃, —COOH, or —CH₂OCH₃;
R₆ is chosen from —H or —CH₃, or R₅ and R₆ together form an azetidine, pyrrolidine, morpholine or piperidine ring, each of which optionally comprises at least one substituent chosen from —CH₃, —OH, —CF₃, or —F;
R₇ is 1, 2, or 3 and independently chosen from —Cl, —F, —CF₃, —CH₃, —OCHF₂, or —OCH₃;
R₅ is chosen from benzene, —CH₃, or tertbutyl;
R₉ is chosen from a cyclohexane optionally comprising at least one —F or —CH₃ substituent and optionally substituted with —O—, or a benzimidazole;
R₁₀ is —C(CH₃)₃, cyclopentyl, —(CH₂) C(CH₃)₃, or a cyclohexane optionally substituted with —N— and optionally comprising at least one substituent chosen from —F or ═O, a cyclopentane optionally comprising an —OCH₃ or an —OH substituent, a tertbutyl, —CF₃, or a cyclopropyl optionally comprising a methyl substituent;
A₁ is chosen from —CH or —N;
A₂ is independently chosen from —CH, —N, or —O; and
A₃ is chosen from —O, CH₂, or CF₂;
X₂ is chosen from —CH₂CONH—, —CH₂—, —CH₂NHCO—, —CH₂NHCOCH₂—, —CH₂NHCO(CH₂)₂—, —NHCO—, —NHCH₂CONH—, —N(CH₃)CH₂CONH—, —CH₂N(CH₃)CO—; —CONH—, —CONHCH₂—, CONHCH₂C(CH₂CH₃)₂—, or —CH₂NH—; and
X₃ is a right-hand substitution of the diazaspiroheptane core chosen from:
an adamantane ring,
a benzofuran,
a phenyl group optionally comprising at least one substituent chosen independently from a halogen. —CH₃, —CF₃, —CHF₂, —OCHF₂, —CN, —OCH(CH₃)₂, —CH₂CH₃, —OCH₂CHF₂, —OCH₃, or cyclopentane,
a cyclohexane optionally comprising a 1,4-CH₂CH₂ bridge,
an imidazole or a benzimidazole, optionally comprising at least one substituent chosen independently from fluorine, chlorine, cyclohexane, —CH₃, —OCHF₂, benzene optionally comprising a halogen substituent, isobutyl, cyclopropyl, tertbutyl, methylpyrazole, —CH(CH₃)₂, or a methyl piperidine,
a pyridine optionally comprising at least one substituent chosen independently from methyl pyrazole, cyclopropyl, or —CH₃,
a naphthalene, or
an isoquinoline optionally comprising at least one halogen substituent.
or a pharmaceutically acceptable salt thereof.

In one aspect, disclosed herein is a compound of Formula (I) having diazaspiroheptane core:

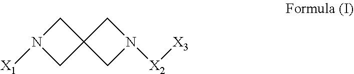

wherein X₁ is a left-hand substitution of the diazaspiroheptane core chosen from:

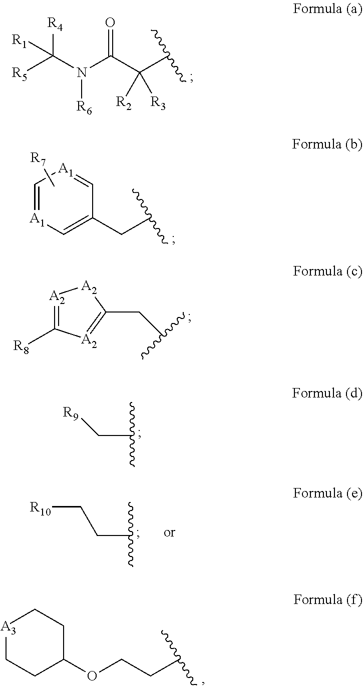

wherein R₁ is chosen from —H, —CH₃, —CH₂OCH₃, —CF₃, —CH₂CH₃, or —(CH₂)₂OCH₃;
R₂ is —H;
R₃ is —H;
R₄ is chosen from —H, or —CH₃ or R₁ and R₄ together form a cyclobutane, a cyclopentane, or an oxetane ring;
R₅ is chosen from —H, —CH₃, —CH₂OH, —COOCH₃, —COOH, or —CH₂OCH₃;
R₆ is chosen from —H or —CH₃, or R₅ and R₆ together form an azetidine, pyrrolidine, morpholine or piperidine ring, each of which optionally comprises at least one substituent chosen from —CH₃, —OH. —CF₃, or —F;
R₇ is 1, 2, or 3, and independently chosen from —Cl, —F, —CF₃, —CH₃, —OCHF₂, or —OCH₃;
R₅ is chosen from benzene, —CH₃, or tertbutyl;
R₉ is chosen from a cyclohexane optionally comprising at least one —F or —CH₃ substituent and optionally substituted with —OH, a benzimidazole;
R₁₀ is a cyclohexane or a piperidine, optionally comprising at least one substituent chosen from —F or ═O, a cyclopentane optionally comprising an —OCH₃ or an —OH substituent, a tertbutyl, —CF₃, or a cyclopropyl optionally comprising a methyl substituent;
A₁ is chosen from —CH or —N;
A₂ is independently chosen from —CH, —N, or —O; and
A₃ is chosen from —O, CH₂, or CF₂;
wherein X₂ is chosen from —CH₂CONH—. —CH₂—, —CH₂NHCO—, —CH₂NHCOCH₂—, —CH₂NHCO(CH₂)₂—, —NHCO—, —NHCH₂CONH—, —N(CH₃)CH₂CONH—, —CH₂N(CH₃)CO—; —CONH—, —CONHCH₂—, CONHCH₂C(CH₂CH₃)₂—, or —CH₂NH—; and
wherein X₃ is a right-hand substitution of the diazaspiroheptane core chosen from:
an adamantane ring,
a benzofuran.
a phenyl group optionally comprising at least one substituent chosen independently from a halogen. —CH₃, —CF₃, —CHF₂, —OCHF₂, —CN, —OCH(CH₃)₂, —CH₂CH₃, —OCH₂CHF₂, —OCH₃, or cyclopentane,
a cyclohexane optionally comprising a 1,4-CH₂CH₂ bridge,
an imidazole or a benzimidazole, optionally comprising at least one substituent chosen independently from chlorine, cyclohexane, —CH₃, benzene optionally comprising a halogen substituent, isobutyl, cyclopropyl, tertbutyl, methylpyrazole, —CH(CH₃)₂, or a methyl piperidine,
a pyridine optionally comprising at least one substituent chosen independently from methyl pyrazole, cyclopropyl, or —CH₃,
a naphthalene, or
an isoquinoline optionally comprising at least one halogen substituent,
   or a pharmaceutically acceptable salt thereof.

In certain embodiments, the compound of Formula (I) is selected from:

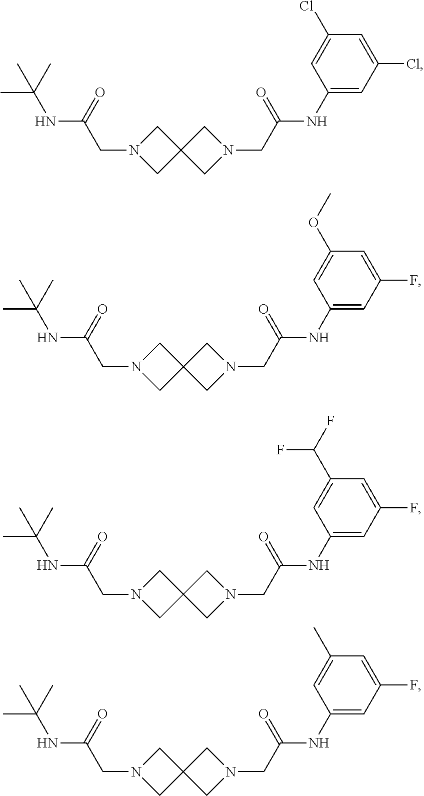 [SMILES: CC(C)(C)NC(=O)CN1CC2(CN(CC(=O)Nc3cc(Cl)cc(Cl)c3)C2)C1 ; COc1cc(F)cc(NC(=O)CN2CC3(C2)CN(CC(=O)NC(C)(C)C)C3)c1 ; CC(C)(C)NC(=O)CN1CC2(CN(CC(=O)Nc3cc(F)cc(C(F)F)c3)C2)C1 ; Cc1cc(F)cc(NC(=O)CN2CC3(C2)CN(CC(=O)NC(C)(C)C)C3)c1]

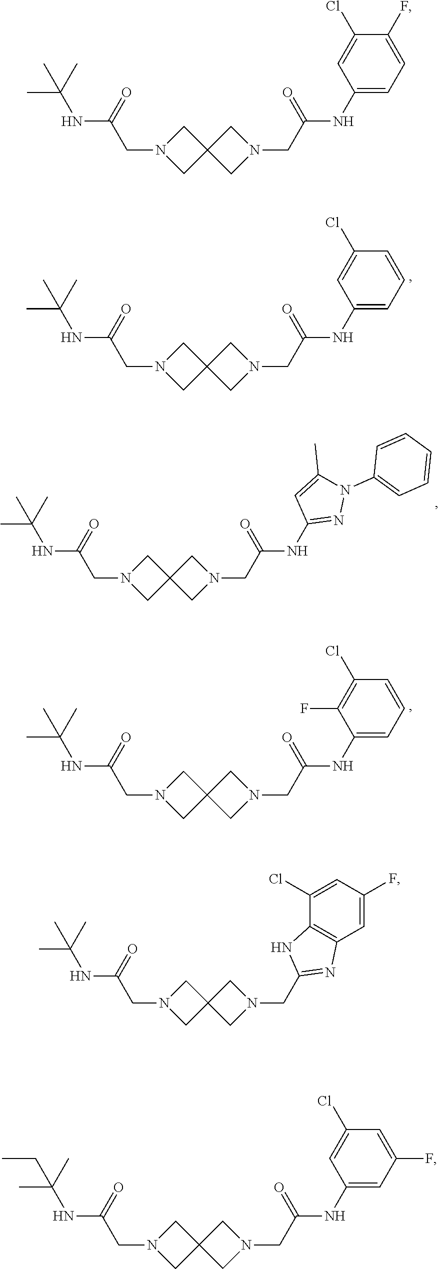 [SMILES: CC(C)(C)NC(=O)CN1CC2(CN(CC(=O)Nc3ccc(F)c(Cl)c3)C2)C1 ; CC(C)(C)NC(=O)CN1CC2(CN(CC(=O)Nc3cccc(Cl)c3)C2)C1 ; Cc1cc(NC(=O)CN2CC3(C2)CN(CC(=O)NC(C)(C)C)C3)nn1-c1ccccc1 ; CC(C)(C)NC(=O)CN1CC2(CN(CC(=O)Nc3cccc(Cl)c3F)C2)C1 ; CC(C)(C)NC(=O)CN1CC2(C1)CN(Cc1nc3cc(F)cc(Cl)c3[nH]1)C2 ; CCC(C)(C)NC(=O)CN1CC2(CN(CC(=O)Nc3cc(F)cc(Cl)c3)C2)C1]

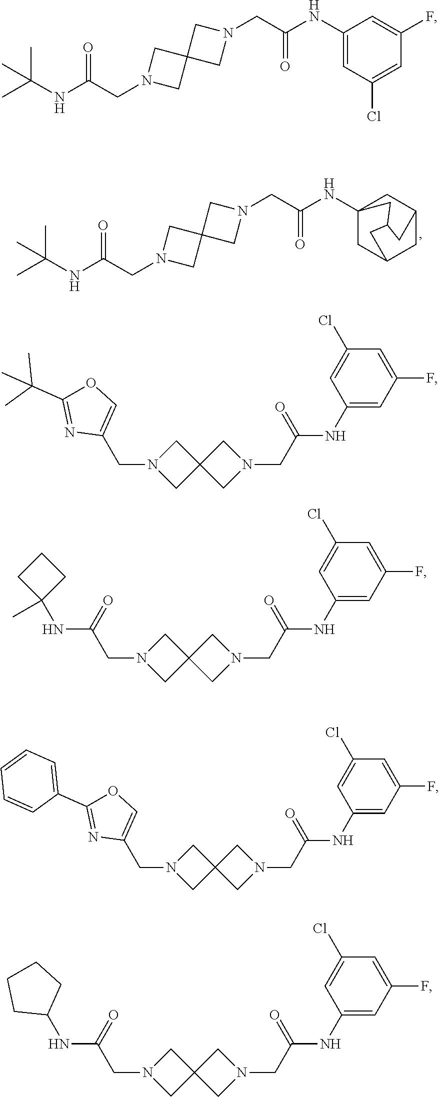 [SMILES: CC(C)(C)NC(=O)CN1CC2(CN(CC(=O)Nc3cc(F)cc(Cl)c3)C2)C1 ; CC(C)(C)NC(=O)CN1CC2(C1)CN(CC(=O)NC13CC4CC(CC(C4)C1)C3)C2 ; CC(C)(C)c1nc(CN2CC3(CN(CC(=O)Nc4cc(F)cc(Cl)c4)C3)C2)co1 ; CC1(NC(=O)CN2CC3(CN(CC(=O)Nc4cc(F)cc(Cl)c4)C3)C2)CCC1 ; O=C(CN1CC2(C1)CN(Cc1coc(-c3ccccc3)n1)C2)Nc1cc(F)cc(Cl)c1 ; O=C(CN1CC2(C1)CN(CC(=O)NC1CCCC1)C2)Nc1cc(F)cc(Cl)c1]

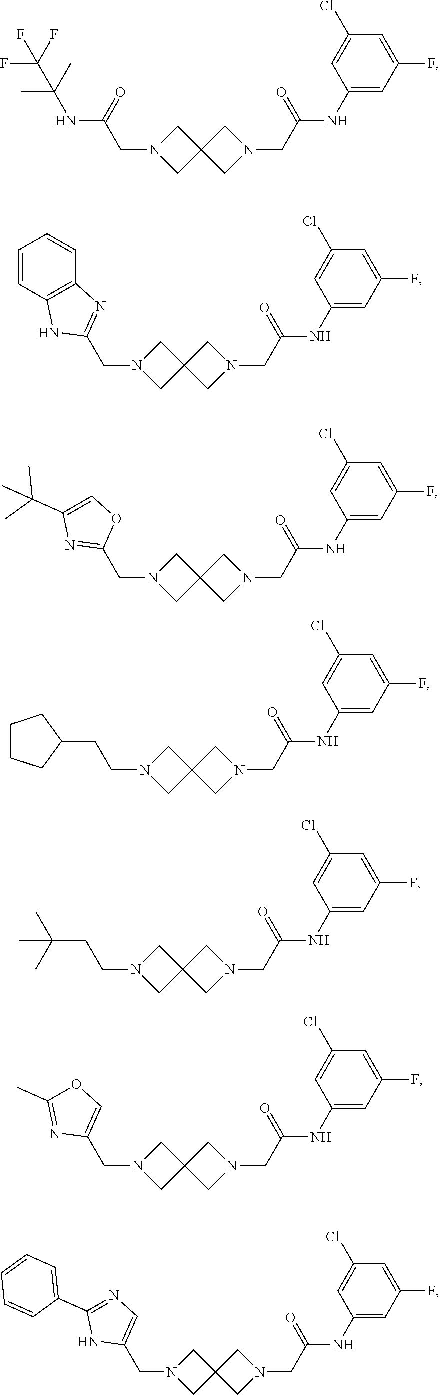

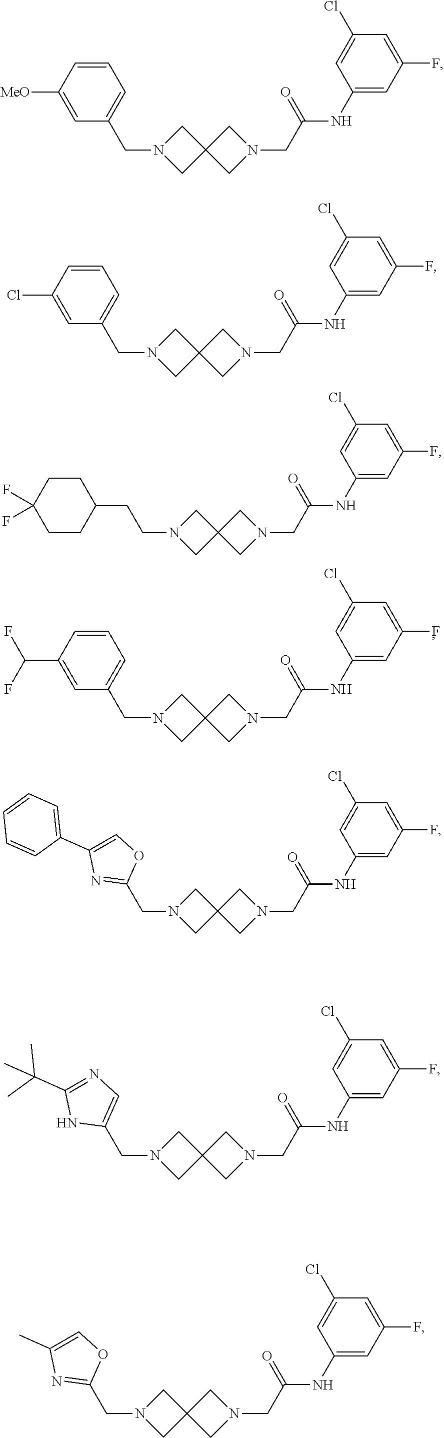

or a pharmaceutically acceptable salt thereof.

In certain embodiments, the compound of Formula (IA) is selected from:

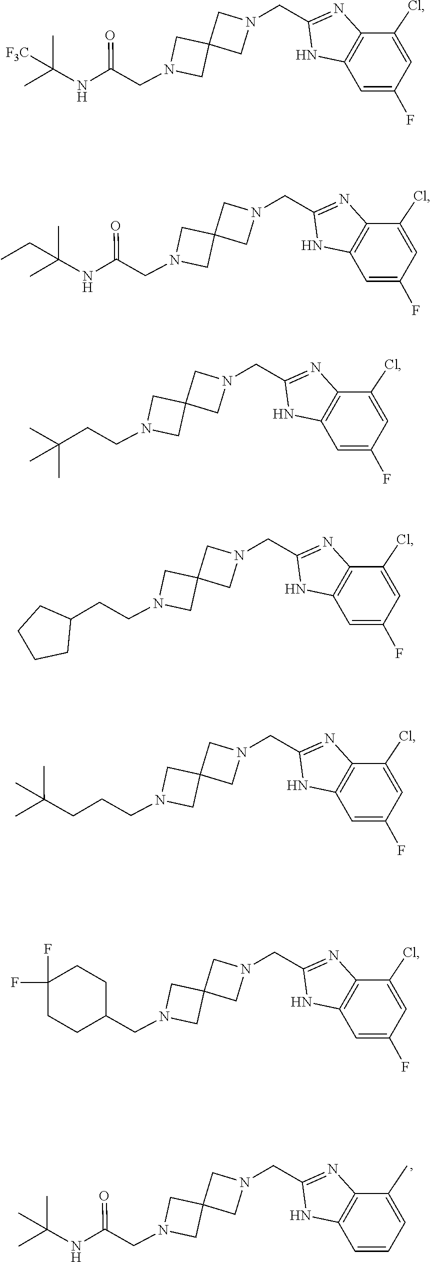

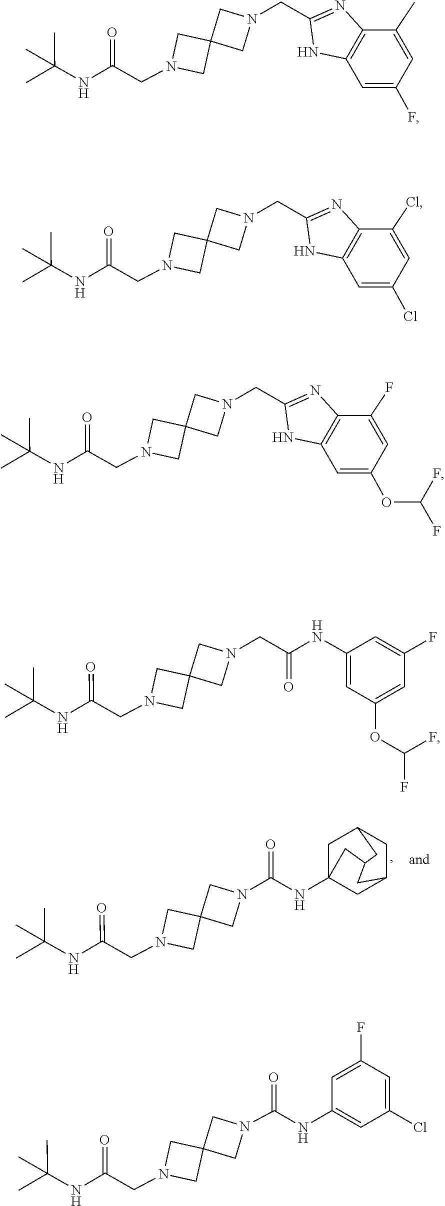

or pharmaceutically acceptable salts thereof.

In certain embodiments of the compounds disclosed herein, X₁ is Formula (a), and in certain embodiments, each of R₁, R₄, and R₅ of Formula (a) is —CH₃. In further embodiments, each of R₂, R₃, and R₆ in Formula (a) is —H. In certain embodiments, X₂ is chosen from —CH₂CONH— or —CH₂—, and in certain embodiments, X₂ is —CONH—. In certain embodiments, R₉ is a cyclohexane with two halogen substituents, such as two fluorine substituents.

In certain embodiments of all aspects, X₃ is an adamantane ring, and in certain embodiments, X₃ is a phenyl. In certain embodiments wherein X₃ is a phenyl, the phenyl group comprises at least one halogen substituent, such as at least two halogen substituents, and in certain embodiments, the at least one halogen is chosen from fluorine or chlorine. In certain aspects disclosed herein, X₃ is a phenyl comprising a fluorine substituent and a chlorine substituent.

In certain embodiments of the compounds disclosed herein, the compound comprises at least one deuterium, and in certain embodiments, the at least one deuterium is in X₁.

In another aspect, disclosed herein are pharmaceutical compositions comprising a compound of Formula (I) or Formula (IA), as disclosed herein, or a pharmaceutically acceptable salt thereof, and a pharmaceutically acceptable carrier. In certain aspects, the pharmaceutical composition further comprises a modified-release polymer, such as hydroxypropyl methylcellulose, ethylcellulose, or a polyacrylate polymer.

In yet another aspect, disclosed herein are methods of treating a disease or condition relating to aberrant function or activity of a T-type calcium channel in a subject in need thereof comprising administering to the subject a therapeutically effective amount of a compound of Formula (I) or Formula (IA), as disclosed herein, or a pharmaceutically acceptable salt thereof or a pharmaceutical composition as disclosed herein. In certain aspects, the disease or condition relating to aberrant function or activity of a T-type calcium channel is a psychiatric disorder, pain, tremor, seizures, epilepsy, or an epilepsy syndrome. In certain embodiments, the disease or condition relating to aberrant function or activity of a T-type calcium channel is tremor, such as essential tremor.

In yet another aspect, disclosed herein is a compound of Formula (I) or Formula (IA), as disclosed herein, or a pharmaceutically acceptable salt thereof or a pharmaceutical composition as disclosed herein for use in treating a disease or condition relating to aberrant function or activity of a T-type calcium channel. In certain aspects, the disease or condition relating to aberrant function or activity of a T-type calcium channel is a psychiatric disorder, pain, tremor, seizures, epilepsy, or an epilepsy syndrome. In certain embodiments, the disease or condition relating to aberrant function or activity of a T-type calcium channel is tremor, such as essential tremor.

### DETAILED DESCRIPTION OF THE DISCLOSURE

Disclosed herein are compounds and compositions useful for preventing and/or treating pain, tremor (e.g., essential tremor), epilepsy or epilepsy syndromes (e.g., absence seizures, juvenile myoclonic epilepsy, or a genetic epilepsy). The compounds and compositions disclosed herein may also be useful for preventing and/or treating psychiatric disorders. Psychiatric disorders may, for example, include, mood disorders such as depression, major depressive disorder, and dysthymic disorder (e.g., mild depression); bipolar disorder (e.g., I and/or II); anxiety disorders (e.g., generalized anxiety disorder (GAD) and social anxiety disorder); stress; post-traumatic stress disorder (PTSD); and compulsive disorders (e.g., obsessive compulsive disorder (OCD)). Methods are also presented that are useful for modulating the function and enhancing the potency of a T-type calcium channel.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used herein, an “effective amount” of a compound refers to an amount sufficient to elicit the desired biological response. As will be appreciated by those of ordinary skill in this art, the effective amount of a compound as disclosed herein may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, the term “refractory” refers to a disease, disorder, or condition that does not readily yield or respond to therapy or treatment or is not controlled by a therapy or treatment. In some embodiments, a disease, disorder, or condition described herein is refractory (e.g., refractory epilepsy or refractory absence seizures) and does not respond to standard therapy or treatment.

As used herein, a “subject” to which administration is contemplated includes, but is not limited to, humans (i.e., a male or female of any age group. e.g., a pediatric subject (e.g., infant, child, or adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human” and “patient” are used interchangeably herein.

The terms “disease.” “disorder.” and “condition” are used interchangeably herein.

As used herein, and unless otherwise specified, the terms “treat.” “treating,” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

As used herein, the term “in some embodiments,” “in other embodiments,” or the like, refers to embodiments of all aspects of the disclosure, unless the context clearly indicates otherwise.

Definitions of specific functional groups and chemical terms are described in more detail below. The chemical elements are identified in accordance with the Periodic Table of the Elements, CAS version. Handbook of Chemistry and Physics. 75th Ed., inside cover, and specific functional groups are generally defined as described therein. Additionally, general principles of organic chemistry, as well as specific functional moieties and reactivity, are described in Thomas Sorrell. Organic Chemistry. 2nd Edition. University Science Books. Sausalito, 2006; Smith and March. March's Advanced Organic Chemistry, 7th Edition, John Wiley & Sons, Inc., New York. 2013; Larock. Comprehensive Organic Transformations, VCH Publishers, Inc., New York, 1989; and Carruthers, Some Modern Methods of Organic Synthesis. 4th Edition. Cambridge University Press, Cambridge, 2004.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereo isomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Race mates and Resolutions (Wiley Interscience. New York, 1981); Wilen et al., Tetrahedron 33:2725 (1977); Eliel. Stereochemistry of Carbon Compounds (McGraw-Hill, NY, 1962); and Wilen. Tables of Resolving Agents and Optical Resolutions p. 268 (E. L. Eliel. Ed., Univ. of Notre Dame Press, Notre Dame. IN 1972). In certain embodiments, the compounds described herein may be individual isomers substantially free of other isomers, or alternatively, as mixtures of various isomers.

As used herein a pure enantiomeric compound is substantially free from other enantiomers or stereo isomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight, or more than 99.9% by weight, of the enantiomer. In certain embodiments, the weights are based upon total weight of all enantiomers or stereo isomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The following terms are intended to have the meanings presented therewith below and are useful in understanding the description and intended scope of the present disclosure. When describing certain aspects of the disclosure, which may include compounds, pharmaceutical compositions containing such compounds, and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

When a range of values is listed, it is intended to encompass each value and sub-range within the range. For example. “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₈, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to, 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2,3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃). 2-propenyl (C₃), 1-butenyl (C₄). 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂). 1-propynyl (C₃). 2-propynyl (C₃), 1-butynyl (C₄), 2-butynyl (C₄), and the like. Examples of C₂_alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₁4 aryl”). In some embodiments, an aryl group has six ring carbon atoms (“C₆ aryl”—e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

“Hetero” when used to describe a compound or a group present on a compound means that one or more carbon atoms in the compound or group have been replaced by a nitrogen, oxygen, or sulfur heteroatom. Hetero may be applied to any of the alkyl groups described above such as alkyl, e.g., heteroalkyl; alkenyl, e.g., heteroalkenyl; alkynyl, e.g., heteroalkynyl; carbocyclyl, e.g., heterocyclyl; aryl, e.g., heteroaryl, and the like having from 1 to 5, and particularly from 1 to 3 heteroatoms.

“Heteroaryl” refers to a radical of a 5-10 membered monocyclic or bicyclic 4n+2 aromatic ring system (e.g., having 6 or 10 n electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋ₛ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C), cyclohexyl (C₆), cyclohexenyl (C), cyclohexadienyl (C₆), and the like. Exemplary C₃₋₅ carbocyclyl groups include, without limitation, the aforementioned C₃₋₆ carbocyclyl groups as well as cycloheptyl (C), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₅ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spire ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The phrase “optionally substituted with —O—” refers to an alkyl or a cycloalkyl group in which a carbon atom in a carbon chain is replaced with an oxygen atom.

The phrase “optionally substituted with —N—” refers to an alkyl or a cycloalkyl group in which a carbon atom in a carbon chain is replaced with a nitrogen atom.

“Cyano” refers to —CN.

“Halo” or “halogen” refers to a fluorine atom (i.e., fluoro or —F), a chlorine atom (i.e., chloro or —Cl), a bromine atom (i.e., bromo or —Br), and an iodine atom (i.e., iodo or —I). In certain embodiments, the halo group is fluoro or chloro.

“Haloalkyl” refers to an alkyl group substituted with one or more halogen atoms.

“Nitro” refers to —NO₂.

In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻). NO₃⁻, ClO₄⁻, OH⁻, H₂PO₄⁻, HSO₄⁻, SO₄⁻, sulfonate ions (e.g., methansulfonate, trifluoromethanesulfonate, p-toluenesulfonate, benzenesulfonate. 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. The general concept of pharmaceutically acceptable salts has been discussed in the art, including, for example. Berge et al., wherein describes pharmaceutically acceptable salts in detail in J Pharmaceutical Sciences (1977) 66: 1-19. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartratc, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “modified-release polymer” refers to a polymer that is used in a formulation (e.g., tablets and capsules) to modify the release rate of the drug upon administration to a subject. For example, a modified-release polymer is used to dissolve a drug over time in order to be released slower and steadier into the bloodstream. For example, a modified-release polymer is a controlled-release polymer. For example, a modified-release polymer or a controlled-release polymer is an HPMC polymer. In some embodiments, a modified-release polymer may include hydrophilic matrix polymers (e.g., hypromellose, hydroxyl-propyl methylcellulose (HPMC)), hydrophobic matrix polymers (e.g., ethyl cellulose, ethocel), or polyacrylate polymers (e.g., Eudragit® RL100, Eudragit® RS100).

The term “diluent” as used herein refers to an excipient used to increase weight and improve content uniformity. For example, diluents include cellulose derivatives (e.g., microcrystalline cellulose), starches (e.g., hydrolyzed starches, and partially pregelatinized starches), anhydrous lactose, lactose monohydrate, di-calcium phosphate (DCP), sugar alcohols (e.g., sorbitol, xylitol and mannitol)).

The term “glidant” as used herein refers to an excipient used to promote powder flow by reducing interparticle friction and cohesion. For example, glidants include fumed silica (e.g., colloidal silicon dioxide), talc, and magnesium carbonate.

The term “lubricant” as used herein refers to an excipient used to prevent ingredients from clumping together and from sticking to the tablet punches or capsule filling machine. Lubricants are also used to ensure that tablet formation and ejection can occur with low friction between the solid and die wall. For example, lubricants include magnesium stearate, calcium stearate, stearic acid, talc, silica, and fats (e.g., vegetable stearin).

The term “coating” as used herein refers to an excipient to protect tablet ingredients from deterioration by moisture in the air and make large or unpleasant-tasting tablets easier to swallow.

The embodiments disclosed herein are not intended to be limited in any manner by the above exemplary listing of chemical groups and substituents.

### Compounds

In one aspect, disclosed herein are compounds and compositions thereof for the modulation of T-type calcium channels, as well as diseases, disorders, or conditions associated with aberrant function thereof (e.g., psychiatric disorders (e.g., mood disorder (e.g., major depressive disorder)); pain; tremor, such as essential tremor; epilepsy or an epilepsy seizure, such as absence seizures, juvenile myoclonic epilepsy, status epilepsy, or a genetic epilepsy.

In some embodiments, the compounds disclosed herein comprise a compound of Formula (IA) with a diazaspiroheptane core:

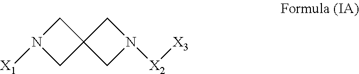

wherein X₁ is a left-hand substitution of the diazaspiroheptane core chosen from:

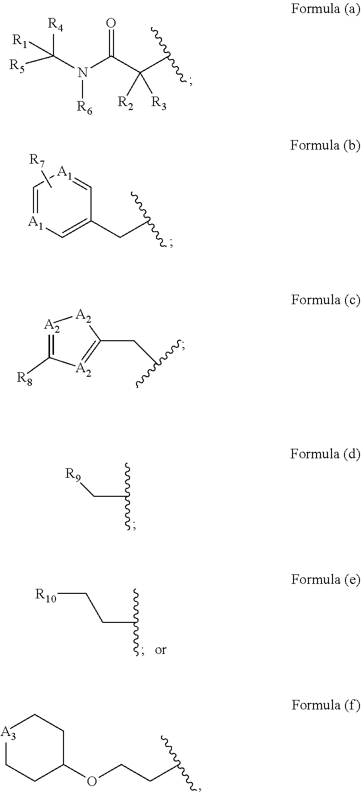

wherein R₁ is chosen from —H, —CH₃, —CH₂OCH₃, —CF₃, —CH₂CH₃, or —(CH₂)₂OCH₃;
R₂ is —H;
R₃ is —H;
R₄ is chosen from —H or —CH₃, or R₁ and R₄ together form a cyclopropane, a cyclobutane, a cyclopentane, or an oxetane ring;
R₅ is chosen from —H, —CH₃, —CF₃, —CH₂OH, —COOCH₃, —COOH, or —CH₂OCH₃;
R₆ is chosen from —H or —CH₃, or R₅ and R₆ together form an azetidine, pyrrolidine, morpholine or piperidine ring, each of which optionally comprises at least one substituent chosen from —CH₃, —OH, —CF₃, or —F;
R₇ is 1, 2, or 3 and independently chosen from —Cl, —F, —CF₃, —CH₃, —OCHF₂, or —OCH₃;
R₅ is chosen from benzene, —CH₃, or tertbutyl;
R₉ is chosen from a cyclohexane optionally comprising at least one —F or —CH₃ substituent and optionally substituted with —O—, or a benzimidazole;
R₁₀ is —C(CH₃)₃, cyclopentyl, —(CH₂) C(CH₃)₃, or a cyclohexane optionally substituted with —N— and optionally comprising at least one substituent chosen from —F or ═O, a cyclopentane optionally comprising an —OCH₃ or an —OH substituent, a tertbutyl, —CF₃, or a cyclopropyl optionally comprising a methyl substituent;
A₁ is chosen from —CH or —N;
A₂ is independently chosen from —CH, —N, or —O; and
A₃ is chosen from —O, CH₂, or CF₂;
X₂ is chosen from —CH₂CONH—, —CH₂—, —CH₂NHCO—, —CH₂NHCOCH₂—, —CH₂NHCO(CH₂)₂—, —NHCO—, —NHCH₂CONH—, —N(CH₃)CH₂CONH—, —CH₂N(CH₃)CO—; —CONH—, —CONHCH₂—, CONHCH₂C(CH₂CH₃)₂—, or —CH₂NH—; and
X₃ is a right-hand substitution of the diazaspiroheptane core chosen from:
an adamantane ring,
a benzofuran,
a phenyl group optionally comprising at least one substituent chosen independently from a halogen. —CH₃, —CF₃, —CHF₂, —OCHF₂, —CN, —OCH(CH₃)₂, —CH₂CH₃, —OCH₂CHF₂, —OCH₃, or cyclopentane,
a cyclohexane optionally comprising a 1,4-CH₂CH₂ bridge,
an imidazole or a benzimidazole, optionally comprising at least one substituent chosen independently from fluorine, chlorine, cyclohexane, —CH₃, —OCHF₂, benzene optionally comprising a halogen substituent, isobutyl, cyclopropyl, tertbutyl, methylpyrazole, —CH(CH₃)₂, or a methyl piperidine,
a pyridine optionally comprising at least one substituent chosen independently from methyl pyrazole, cyclopropyl, or —CH₃,
a naphthalene, or
an isoquinoline optionally comprising at least one halogen substituent.
or a pharmaceutically acceptable salt thereof.

In some embodiments, the compounds disclosed herein comprise a compound of Formula (I) with a diazaspiroheptane core:

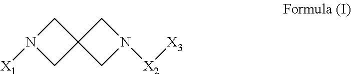

wherein X₁ is a left-hand substitution of the diazaspiroheptane core chosen from:

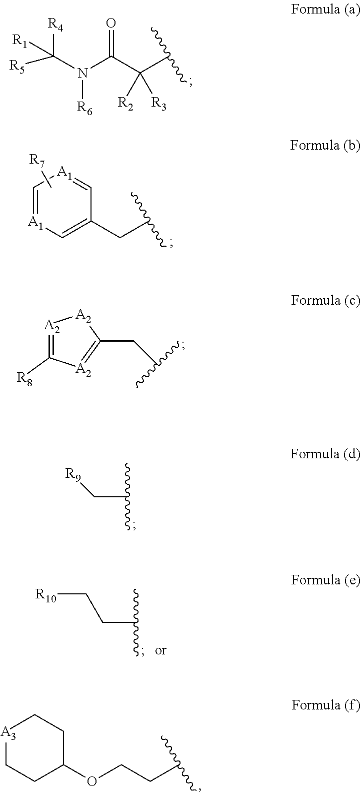

wherein R₁ is chosen from —H, —CH₃, —CH₂OCH₃, —CF₃, —CH₂CH₃, or —(CH₂)₂OCH₃;
R₂ is —H;
R₃ is —H;
R₄ is chosen from —H or —CH₃, or R₁ and R₄ together form a cyclobutane, a cyclopentane, or an oxetane ring;
R₅ is chosen from —H, —CH₃, —CH₂OH, —COOCH₃, —COOH, or —CH₂OCH₃;
R₆ is chosen from —H or —CH₃, or R₅ and R₆ together form an azetidine, pyrrolidine, morpholine or piperidine ring, each of which optionally comprises at least one substituent chosen from —CH₃, —OH. —CF₃, or —F;
R₇ is 1, 2, or 3 and independently chosen from —Cl, —F, —CF₃, —CH₃, —OCHF₂, or —OCH₃;
R₅ is chosen from benzene, —CH₃, or tertbutyl;
R₉ is chosen from a cyclohexane optionally comprising at least one —F or —CH₃ substituent and optionally substituted with —O—, or a benzimidazole;
R₁₀ is a cyclohexane optionally substituted with —N— and optionally comprising at least one substituent chosen from —F or ═O, a cyclopentane optionally comprising an —OCH₃ or an —OH substituent, a tertbutyl, —CF₃, or a cyclopropyl optionally comprising a methyl substituent;
A₁ is chosen from —CH or —N;
A₂ is independently chosen from —CH, —N, or —O; and
A₃ is chosen from —O, CH₂, or CF₂;
X₂ is chosen from —CH₂CONH—, —CH₂—, —CH₂NHCO—, —CH₂NHCOCH₂—, —CH₂NHCO(CH₂)₂—, —NHCO—, —NHCH₂CONH—, —N(CH₃)CH₂CONH—, —CH₂N(CH₃)CO—; —CONH—, —CONHCH₂—, CONHCH₂C(CH₂CH₃)₂—, or —CH₂NH—; and
X₃ is a right-hand substitution of the diazaspiroheptane core chosen from:
an adamantane ring,
a benzofuran,
a phenyl group optionally comprising at least one substituent chosen independently from a halogen, —CH₃, —CF₃, —CHF₂, —OCHF₂, —CN, —OCH(CH₃)₂, —CH₂CH₃, —OCH₂CHF₂, —OCH₃, or cyclopentane,
a cyclohexane optionally comprising a 1,4-CH₂CH₂ bridge,
an imidazole or a benzimidazole, optionally comprising at least one substituent chosen independently from chlorine, cyclohexane, —CH₃, benzene optionally comprising a halogen substituent, isobutyl, cyclopropyl, tertbutyl, methylpyrazole. —CH(CH₃)₂, or a methyl piperidine.
a pyridine optionally comprising at least one substituent chosen independently from methyl pyrazole, cyclopropyl, or —CH₃,
a naphthalene, or
an isoquinoline optionally comprising at least one halogen substituent,
or a pharmaceutically acceptable salt thereof.

In certain embodiments. X₁ is Formula (a) and R₂, R₃, and R₆ are —H, and in certain embodiments. R₁, R₅, and R₆ are —CH₃. In certain embodiments, X₂ is chosen from —CH₂CONH— or —CH₂—, and in certain embodiments, X₂ is X₂ is —CH₂CONH—. In certain embodiments, X₃ is an adamantane ring, and in certain embodiments, X₃ is a phenyl group, for example a phenyl group comprising at least two halogen substituents. In certain embodiments, the halogen substituents may be chosen from chlorine, fluorine, or any combination thereof. In certain embodiments, R₉ is a cyclohexane with two halogen substituents, such as two fluorine substituents.

In certain embodiments, the compounds disclosed herein comprise a compound of Formula (I), wherein the compound has a structure as set forth below:

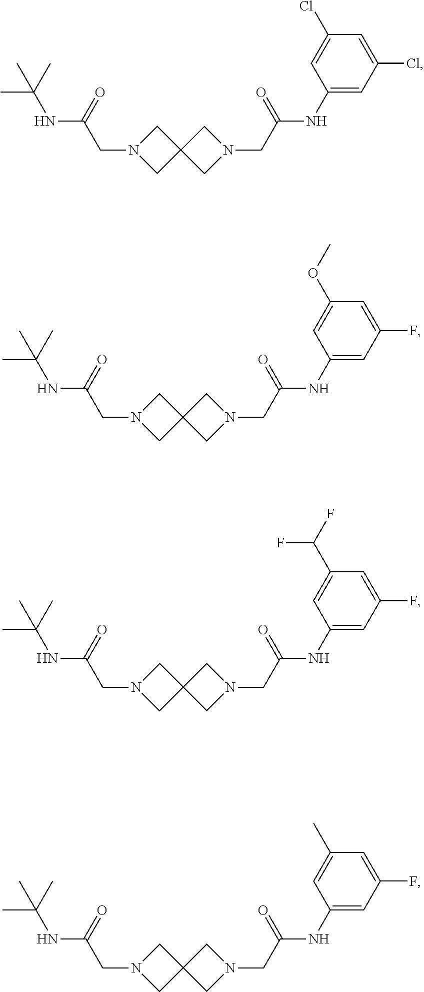 [SMILES: CC(C)(C)NC(=O)CN1CC2(CN(CC(=O)Nc3cc(Cl)cc(Cl)c3)C2)C1 ; COc1cc(F)cc(NC(=O)CN2CC3(C2)CN(CC(=O)NC(C)(C)C)C3)c1 ; CC(C)(C)NC(=O)CN1CC2(CN(CC(=O)Nc3cc(F)cc(C(F)F)c3)C2)C1 ; Cc1cc(F)cc(NC(=O)CN2CC3(C2)CN(CC(=O)NC(C)(C)C)C3)c1]

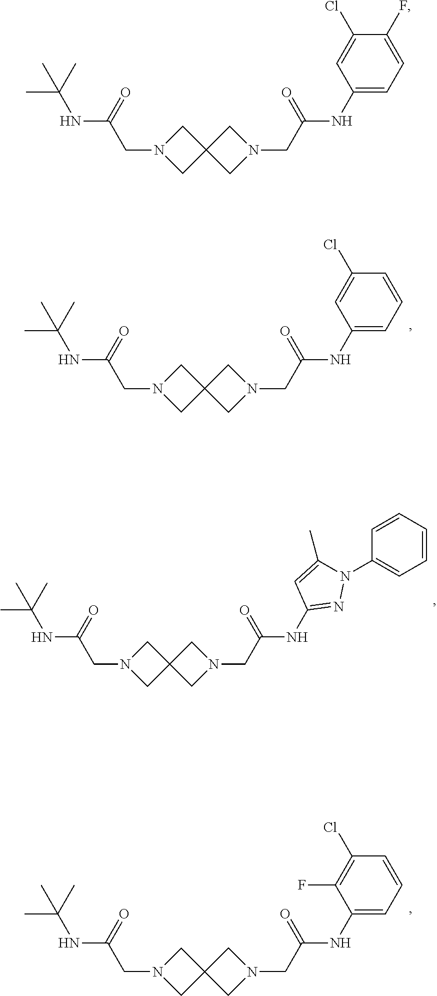 [SMILES: CC(C)(C)NC(=O)CN1CC2(CN(CC(=O)Nc3ccc(F)c(Cl)c3)C2)C1 ; CC(C)(C)NC(=O)CN1CC2(CN(CC(=O)Nc3cccc(Cl)c3)C2)C1 ; Cc1cc(NC(=O)CN2CC3(C2)CN(CC(=O)NC(C)(C)C)C3)nn1-c1ccccc1 ; CC(C)(C)NC(=O)CN1CC2(CN(CC(=O)Nc3cccc(Cl)c3F)C2)C1]

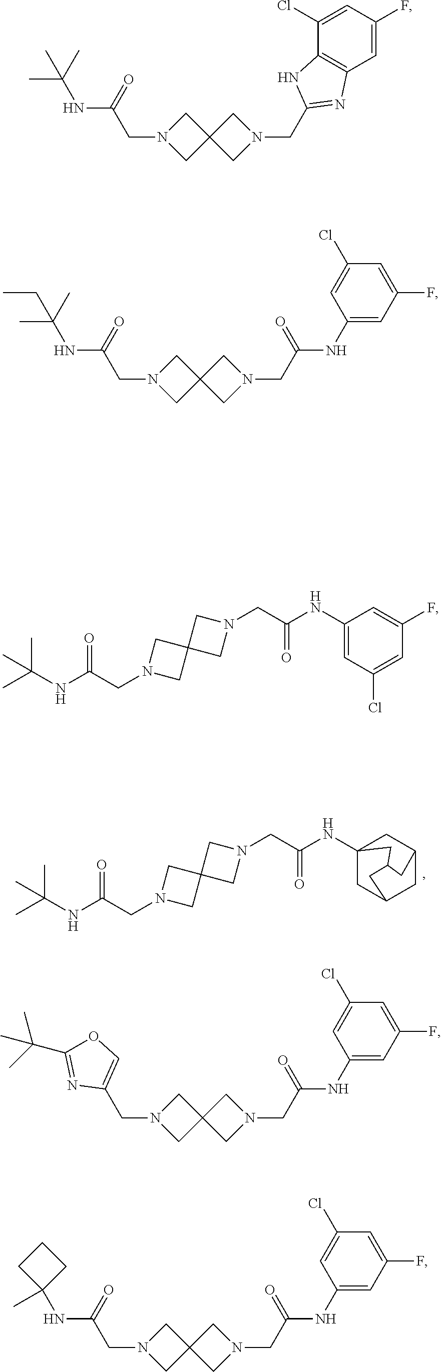 [SMILES: CC(C)(C)NC(=O)CN1CC2(C1)CN(Cc1nc3cc(F)cc(Cl)c3[nH]1)C2 ; CCC(C)(C)NC(=O)CN1CC2(CN(CC(=O)Nc3cc(F)cc(Cl)c3)C2)C1 ; CC(C)(C)NC(=O)CN1CC2(CN(CC(=O)Nc3cc(F)cc(Cl)c3)C2)C1 ; CC(C)(C)NC(=O)CN1CC2(C1)CN(CC(=O)NC13CC4CC(CC(C4)C1)C3)C2 ; CC(C)(C)c1nc(CN2CC3(CN(CC(=O)Nc4cc(F)cc(Cl)c4)C3)C2)co1 ; CC1(NC(=O)CN2CC3(CN(CC(=O)Nc4cc(F)cc(Cl)c4)C3)C2)CCC1]

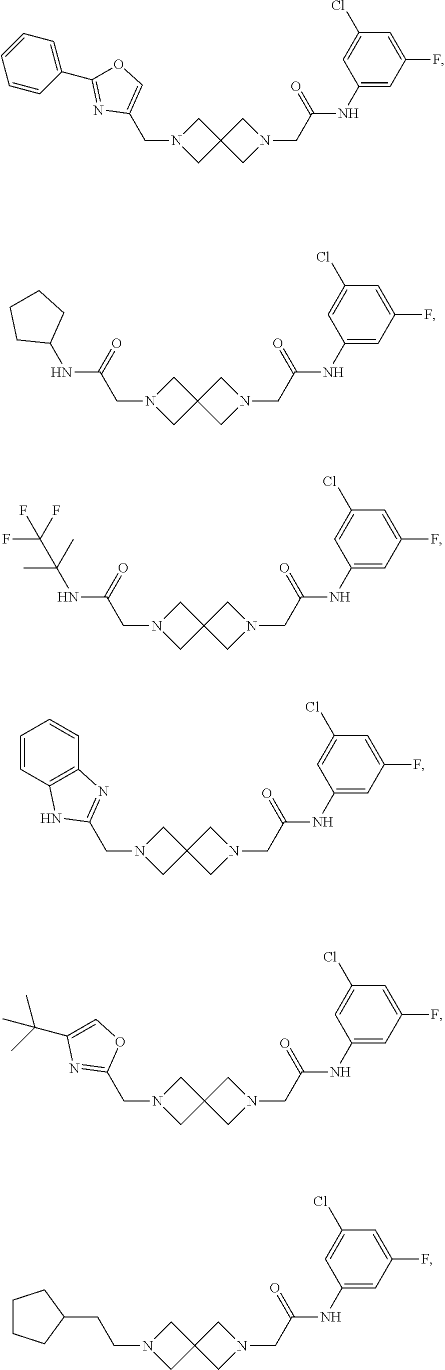 [SMILES: O=C(CN1CC2(C1)CN(Cc1coc(-c3ccccc3)n1)C2)Nc1cc(F)cc(Cl)c1 ; O=C(CN1CC2(C1)CN(CC(=O)NC1CCCC1)C2)Nc1cc(F)cc(Cl)c1 ; CC(C)(NC(=O)CN1CC2(CN(CC(=O)Nc3cc(F)cc(Cl)c3)C2)C1)C(F)(F)F ; O=C(CN1CC2(C1)CN(Cc1nc3ccccc3[nH]1)C2)Nc1cc(F)cc(Cl)c1 ; CC(C)(C)c1coc(CN2CC3(CN(CC(=O)Nc4cc(F)cc(Cl)c4)C3)C2)n1 ; O=C(CN1CC2(CN(CCC3CCCC3)C2)C1)Nc1cc(F)cc(Cl)c1]

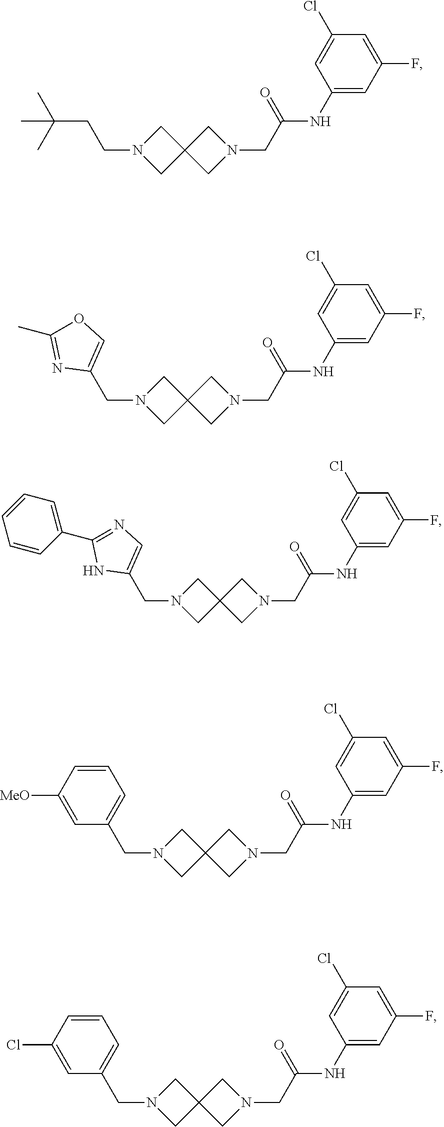 [SMILES: CC(C)(C)CCN1CC2(C1)CN(CC(=O)Nc1cc(F)cc(Cl)c1)C2 ; Cc1nc(CN2CC3(CN(CC(=O)Nc4cc(F)cc(Cl)c4)C3)C2)co1 ; O=C(CN1CC2(C1)CN(Cc1cnc(-c3ccccc3)[nH]1)C2)Nc1cc(F)cc(Cl)c1 ; COc1cccc(CN2CC3(CN(CC(=O)Nc4cc(F)cc(Cl)c4)C3)C2)c1 ; O=C(CN1CC2(C1)CN(Cc1cccc(Cl)c1)C2)Nc1cc(F)cc(Cl)c1]

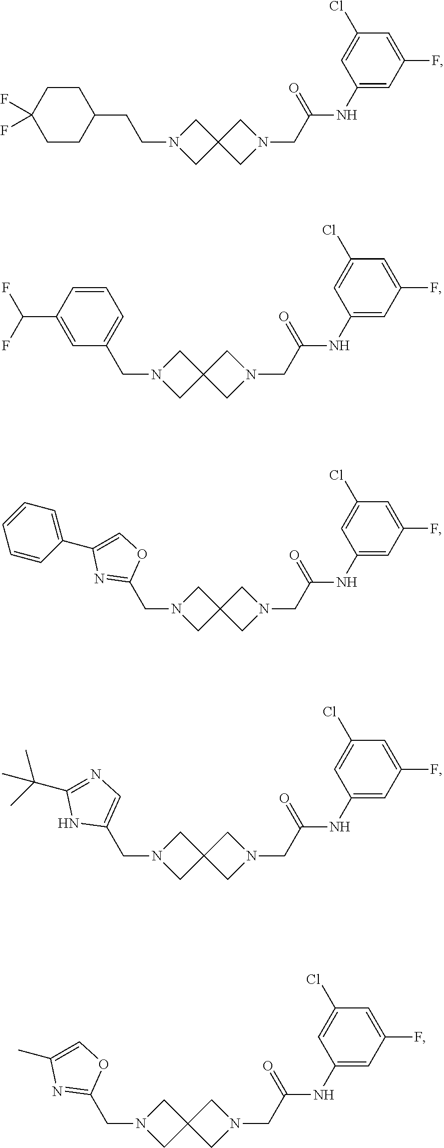 [SMILES: O=C(CN1CC2(CN(CCC3CCC(F)(F)CC3)C2)C1)Nc1cc(F)cc(Cl)c1 ; O=C(CN1CC2(C1)CN(Cc1cccc(C(F)F)c1)C2)Nc1cc(F)cc(Cl)c1 ; O=C(CN1CC2(C1)CN(Cc1nc(-c3ccccc3)co1)C2)Nc1cc(F)cc(Cl)c1 ; CC(C)(C)c1ncc(CN2CC3(CN(CC(=O)Nc4cc(F)cc(Cl)c4)C3)C2)[nH]1 ; Cc1coc(CN2CC3(CN(CC(=O)Nc4cc(F)cc(Cl)c4)C3)C2)n1]

or pharmaceutically acceptable salts thereof.

In another embodiment, the compounds disclosed herein comprise a compound of Formula (IA), wherein the compound has a structure as set forth below:

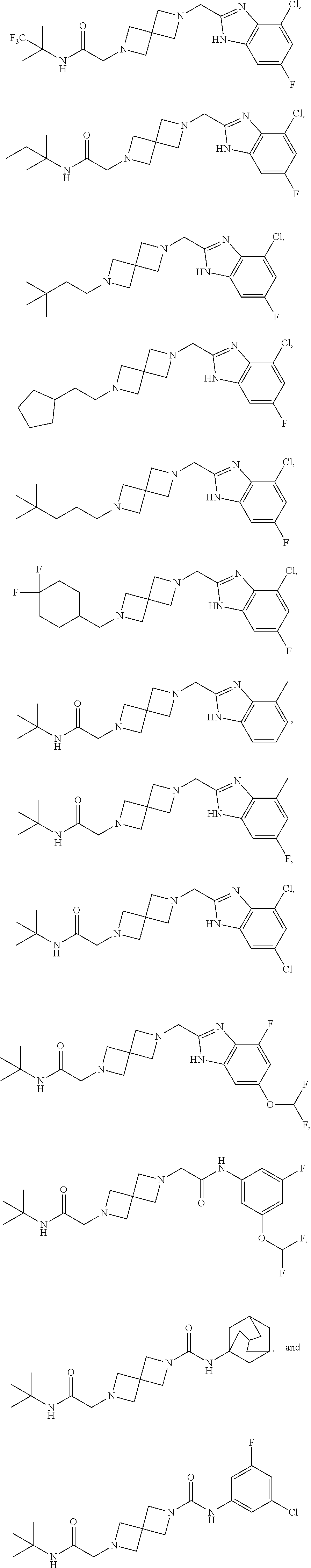

or pharmaceutically acceptable salts thereof.

### Compositions

In one aspect, the compound of Formula (I) or Formula (IA), as disclosed herein, or a pharmaceutically acceptable salt thereof, may be in a pharmaceutical composition, such as in a dosage form. As used herein, the terms pharmaceutical composition and dosage form may be used interchangeably.

In some embodiments, a composition that can be used in a method described herein may be a pharmaceutical composition comprising the compound of Formula (I) or Formula (IA), as disclosed herein, or a pharmaceutically acceptable salt thereof, and an excipient that functions to modify the release rate of the compound of Formula (I) or Formula (IA), as disclosed herein, or a pharmaceutically acceptable salt thereof. In some embodiments, the pharmaceutical composition may be a swellable core technology formulation.

In certain embodiments, a dosage form that can be used in a method described herein may be a dosage form, such as an oral dosage form, comprising the compound of Formula (I) or Formula (IA), as disclosed herein, or a pharmaceutically acceptable salt thereof, and a modified-release polymer (e.g., a controlled-release polymer, hydrophilic matrix polymers, e.g., an HPMC polymer, hydrophobic matrix polymers (e.g., ethyl cellulose, ethocel), or polyacrylate polymers (e.g., Eudragit® RL100, Eudragit® RS100)), in an amount sufficient to modify the release rate of the compound of Formula (I) or Formula (IA), as disclosed herein, or a pharmaceutically acceptable salt thereof.

In some embodiments, the dosage form may comprises from about 0.9% by weight to about 40% by weight (e.g., from about 0.9% by weight to about 30%, from about 1% by weight to about 25% by weight, from about 2% by weight to about 25% by weight, from about 3% by weight to about 20% by weight, from about 4% by weight to about 20% by weight, from about 5% by weight to about 20% by weight, from about 5% by weight to about 15% by weight, from about 5% by weight to about 10% by weight, or about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 40% by weight) of the compound of Formula (I) or Formula (IA), as disclosed herein, or a pharmaceutically acceptable salt thereof. In some embodiments, the dosage form comprises about 30% by weight to about 40% by weight of the compound of Formula (I) or Formula (IA), as disclosed herein, or a pharmaceutically acceptable salt thereof.

In some embodiments, the dosage form may comprise from about 4% by weight to about 25% by weight of the compound of Formula (I) or Formula (IA), as disclosed herein, or a pharmaceutically acceptable salt thereof, such as, for example from about 19% to about 20%, from about 21% to about 22%, from about 4% to about 15%, from about 4% to about 10%, from about 4% to about 5%, from about 5% to about 6%, or from about 9% to about 10%, by weight.

In some embodiments, a dosage form that can be used in a method described herein may be a dosage form or composition comprising from about 0 mg to about 60 mg (e.g., about 1 mg to about 20 mg, about 5 mg to about 25 mg, about 10 mg to about 30 mg, about 15 mg to about 35 mg, about 20 mg to about 40 mg, about 25 mg to about 55 mg or about 30 mg to about 60 mg of the compound of Formula (I) or Formula (IA), as disclosed herein, or a pharmaceutically acceptable salt thereof. In other embodiments, a dosage form that can be used in a method described herein may be a dosage form or composition comprising from about 1 mg to about 60 mg (e.g., about 5 mg, about 10 mg, about 15 mg, about 20 mg, about 25 mg, about 30 mg, about 35 mg, about 40 mg, about 45 mg, about 50 mg, about 55 mg, or about 60 mg) of the compound of Formula (I) or Formula (IA), as disclosed herein, or a pharmaceutically acceptable salt thereof, and a modified-release polymer (e.g., a controlled-release polymer, hydrophilic matrix polymers, e.g., an HPMC polymer, hydrophobic matrix polymers (e.g., ethyl cellulose, ethocel), or polyacrylate polymers (e.g., Eudragit® RL100, Eudragit® RS100)), for example, in an amount sufficient to modify the release rate of the compound of Formula (I) or Formula (IA), as disclosed herein, or a pharmaceutically acceptable salt thereof upon an administration to the subject.

In other embodiments, the dosage form comprises from about 4 mg to about 6 mg (e.g., about 5 mg) of the compound of Formula (I) or Formula (IA), as disclosed herein, or a pharmaceutically acceptable salt thereof. In certain embodiments, the dosage form comprises from about 15 mg to about 45 mg (e.g., about 20 mg) of the compound of Formula (I) or Formula (IA), as disclosed herein, or a pharmaceutically acceptable salt thereof, such as from about 5 mg to about 15 mg (e.g., about 10 mg), from about 15 mg to about 25 mg, from about 25 mg to about 35 mg (e.g., about 30 mg), or from about 35 mg to about 45 mg (e.g., about 40 mg).

In some embodiments, the dosage form comprises from about 55 mg to 65 mg of a modified-release polymer (e.g., an HPMC polymer). In some embodiments, the dosage form comprises from about 10% by weight to about 70% by weight of the modified-release polymer (e.g., an HPMC polymer). In some embodiments, the dosage form comprises from about 50% by weight to about 60% by weight of the modified-release polymer (e.g., an HPMC polymer).

In some embodiments, the dosage form further comprises a diluent. In some embodiments, the diluent comprises microcrystalline cellulose. In some embodiments, the dosage form comprises from about 15 mg to 40 mg (e.g., from about 15 mg to about 25 mg, from about 20 mg to about 25 mg, from about 25 mg to about 30 mg, from about 30 mg to about 40 mg) microcrystalline cellulose. In some embodiments, the dosage form comprises from about 15 mg to about 25 mg microcrystalline cellulose. In some embodiments, the dosage form comprises from about 30 mg to about 40 mg microcrystalline cellulose. In some embodiments, the dosage form comprises from about 15% to about 35% by weight (e.g., from about 15% to about 20%, from about 20% to about 25%, from 25% to about 30%, or from 30% to about 35% by weight) microcrystalline cellulose.

In some embodiments, the dosage form further comprises a glidant. In some embodiments, the glidant comprises colloidal silicon dioxide. In some embodiments, the dosage form further comprises a lubricant. In some embodiments, the lubricant comprises magnesium stearate. In some embodiments, the dosage form further comprises a coating.

In some embodiments, about 80% of the compound of Formula (I) or Formula (IA), as disclosed herein, or a pharmaceutically acceptable salt thereof, is released within 7 hours upon administration to a subject. In certain embodiments, about 80% of the compound of Formula (I) or Formula (IA), as disclosed herein, or a pharmaceutically acceptable salt thereof, is released in 7 hours using USP apparatus type-I, media containing 900 mL 0.1 M HCl, and a paddle speed of 100 rpm.

In some embodiments, the dosage form, upon administration to a subject, has a reduced C, value than a reference oral dosage form (e.g., a dosage form without any intended release rate profile (e.g., without a modified release rate profile or a dosage form that does not have a modified-release polymer, e.g., an HPMC polymer)). In some embodiments, the dosage form, upon administration to a subject, has a greater t, value than a reference oral dosage form (e.g., a dosage form without any intended release rate profile (e.g., without a modified release rate profile or a dosage form that does not have a modified-release polymer, e.g., an HPMC polymer)).

In other embodiments, the dosage form is administered to a patient once daily. In certain embodiments, the dosage form is administered to a patient twice daily. In some embodiments, the dosage form is a tablet. In other embodiments, the dosage form is a capsule. In certain embodiments, the dosage form is a suspension.

In some embodiments, a dosage form that can be used in a method described herein may be an oral dosage form (e.g., particulate) comprising from about 15 mg to 25 mg of the compound of Formula (I) or Formula (IA) or a pharmaceutically acceptable salt thereof; and from about 55 mg to 65 mg of an HPMC polymer.

In other embodiments, a dosage form that can be used in a method described herein may be an oral dosage form (e.g., particulate) comprising from about 14% by weight to about 25% by weight of the compound of Formula (I) or Formula (IA) or a pharmaceutically acceptable salt thereof; and from about 53% to about 64% by weight of an HPMC polymer.

In certain embodiments, a dosage form that can be used in a method described herein may be an oral dosage form (e.g., particulate) comprising from about 3 mg to 8 mg of the compound of Formula (I) or Formula (IA) or a pharmaceutically acceptable salt thereof; and from about 55 mg to 65 mg of an HPMC polymer.

In some embodiments, a dosage form that can be used in a method described herein may be an oral dosage form (e.g., particulate) comprising from about 3% by weight to about 8% by weight of the compound of Formula (I) or Formula (IA) or a pharmaceutically acceptable salt thereof; and from about 53% to about 64% by weight of an HPMC polymer.

In other embodiments, a dosage form that can be used in a method described herein may be an oral (e.g., particulate) composition comprising the compound of Formula (I) or Formula (IA) or a pharmaceutically acceptable salt thereof; and a modified-release polymer (e.g., a controlled-release polymer, e.g., an HPMC polymer as a hydrophilic matrix polymer).

In some embodiments, the composition comprises from about 0.9% by weight to about 40% by weight of the compound of Formula (I) or Formula (IA) or a pharmaceutically acceptable salt thereof. In some embodiments, the composition comprises from about 14% to about 25%, about 19% to about 20%, about 21% to about 22%, about 4% to about 15%, about 4% to about 10%, about 4% to about 5%, about 5% to about 6%, or about 9% to about 10%, by weight of the compound of Formula (I) or Formula (IA) or a pharmaceutically acceptable salt thereof.

In some embodiments, the composition comprises from about 1 mg to about 60 mg (e.g., about 5 mg, about 10 mg, about 15 mg, about 20 mg, about 25 mg, about 30 mg, about 35 mg, about 40 mg, about 45 mg, about 50 mg, about 55 mg, or about 60 mg) of the compound of Formula (I) or Formula (IA) or a pharmaceutically acceptable salt thereof. In certain embodiments, the composition comprises from about 4 mg to about 6 mg (e.g., about 5 mg) of the compound of Formula (I) or Formula (IA) or a pharmaceutically acceptable salt thereof. In other embodiments, the composition comprises from about 15 mg to about 25 mg (e.g., about 20 mg) of the compound of Formula (I) or Formula (IA) or a pharmaceutically acceptable salt thereof.

In some embodiments, the composition comprises a diluent. In some embodiments, the diluent comprises microcrystalline cellulose. In other embodiments, the composition comprises from about 15 mg to 40 mg (e.g., from about 15 mg to about 25 mg, from about 20 mg to about 25 mg, from about 25 mg to about 30 mg, or from about 30 mg to about 40 mg) microcrystalline cellulose. In some embodiments, the composition comprises from about 15% to about 35% by weight (e.g., from about 15% to about 20%, from about 20% to about 25%, from 25% to about 30%, or from 30% to about 35% by weight) microcrystalline cellulose.

In some embodiments, the composition comprises from about 15 mg to about 25 mg microcrystalline cellulose. In some embodiments, the composition comprises from about 30 mg to about 40 mg microcrystalline cellulose. In some embodiments, the composition further comprises a glidant. In some embodiments, the glidant comprises colloidal silicon dioxide. In some embodiments, the composition further comprises a lubricant. In some embodiments, the lubricant comprises magnesium stearate. In some embodiments, the composition further comprises a coating. In some embodiments, the compound of Formula (I) or Formula (IA) or a pharmaceutically acceptable salt thereof, is stable within the formulation at about 25° C. at 60% relative humidity for at least 24 months. In some embodiments, the compound is stable at about 25° C. at 60% relative humidity for at least 36 months. In some embodiments, the compound is stable at about 25° C. at 60% relative humidity for at least 48 months. In other embodiments, the compound is stable at about 25° C. at 60% relative humidity for at least 60 months. In some embodiments, the compound is stable at about 40° C. at 75% relative humidity for at least 6 months.

### Immediate Release Formulations

In some embodiments, a dosage form or composition that can be used in the methods described herein may be a dosage form or composition comprising the compound of Formula (I) or Formula (IA) or a pharmaceutically acceptable salt thereof, where the compound Formula (I) or Formula (IA) or a pharmaceutically acceptable salt thereof is released immediately upon an administration to the subject.

In other embodiments, a dosage form that can be used in a method described herein may be an oral capsule for immediate release comprising from about 15 mg to about 20 mg of the compound of Formula (I) or Formula (IA) or a pharmaceutically acceptable salt thereof; and from about 75 mg to 85 mg diluent; from about 2 mg to 10 mg binder; from about 1% to about 5% disintegrant; and from about 0.1 mg to 5 mg lubricant.

### Administrations

In one aspect, the compounds, compositions, dosage forms, and the like described herein may be administered to a subject. In some embodiments, the dosage form is administered to the subject more than once a day (e.g., twice a day, three times a day, or four times a day).

In some embodiments, the dosage form is administered to the subject once a day (e.g., one 20 mg tablet once a day, two 20 mg tablets once a day, or three 20 mg tablets once a day). In some embodiments, the dosage form is administered to the subject twice a day (e.g., one 10 mg tablet twice a day, one 20 mg tablet twice a day, two 20 mg tablets twice a day, three 20 mg tablets twice a day). In some embodiments, the dosage form is administered to the subject every other day. In certain embodiments, about 1 mg to about 60 mg, such as about 20 mg to about 40 mg, of the compound of Formula (I) or Formula (IA) or a pharmaceutically acceptable salt thereof, is administered to the subject daily. In other embodiments, about 15 mg to 25 mg of the compound of Formula (I) or Formula (IA) or a pharmaceutically acceptable salt thereof is administered to the subject daily. In certain embodiments, about 30 mg to 40 mg of the compound of Formula (I) or Formula (IA) or a pharmaceutically acceptable salt thereof is administered to the subject daily.

Compounds provided in accordance with the present disclosure are usually administered in the form of pharmaceutical compositions. This invention therefore provides pharmaceutical compositions that contain, as the active ingredient, one or more of the compounds described, or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. General techniques for preparing pharmaceutical compositions are disclosed in the pharmaceutical art (see, e.g., Remington's Pharmaceutical Sciences, Mace Publishing Co., Philadelphia, Pa. 17^{th} Ed. (1985); and Modern Pharmaceutics, Marcel Dekker, Inc. 3^{rd} Ed. (G. S. Banker & C. T. Rhodes, Eds.).

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

In some embodiments, the compounds and compositions described herein are administered orally. The compound or a composition thereof may be formulated in a liquid or oral dosage form. Administration may be via capsule or tablet (e.g., an enteric coated tablet), or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of a tablet, pill, powder, lozenge, sachet, elixir, suspension, emulsion, solution, syrup, aerosol (as a solid or in a liquid medium), or ointment containing, for example, up to 10% by weight of the active compound, or capsule (e.g., soft or hard gelatin capsule).

In some embodiments, the compounds and compositions described herein are administered parenterally, e.g., by injection or intravenously. The compound or a composition thereof may be formulated in a liquid dosage form and may include one or more excipients.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions disclosed herein can be formulated so as to provide quick, sustained, or delayed release of the active ingredient after administration to the patient. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the compounds disclosed herein in controlled amounts. The general construction and use of transdermal patches for the delivery of pharmaceutical agents is described in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from 1 mg to 2 g of a compound described herein, and for parenteral administration, preferably from 0.1 to 700 mg of a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound as disclosed herein. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills disclosed herein may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

### Methods of Treating a Disease or Condition Relating to Aberrant Function or Activity of a T-Type Calcium Channel

In one aspect, the present disclosure provides a method of treating a disease or condition relating to aberrant function or activity of a T-type calcium channel in a subject in need thereof, the method comprising administering (e.g., once, twice, three times) daily to the subject a therapeutically effective amount of the compound of Formula (I) or Formula (IA), as disclosed herein, or a pharmaceutically acceptable salt thereof, or a pharmaceutical composition comprising the same. The present disclosure similarly provides a therapeutically effective amount of the compound of Formula (I) or Formula (IA), as disclosed herein, or a pharmaceutically acceptable salt thereof, or a pharmaceutical composition comprising the same, for use in treating a disease or condition relating to aberrant function or activity of a T-type calcium channel in a subject in need thereof.

In typical embodiments, the present disclosure is intended to encompass the compounds disclosed herein, and the pharmaceutically acceptable salts, tautomeric forms, polymorphs, and prodrugs of such compounds. In some embodiments, the present invention includes a pharmaceutically acceptable addition salt, a hydrate of an addition salt, a tautomeric form, a polymorph, an enantiomer, a mixture of enantiomers, a stereo isomer or mixture of stereoisomers (pure or as a racemic or non-racemic mixture) of a compound described herein. In all embodiments of the methods disclosed herein, the compound may be in the form of a composition, including a pharmaceutical composition or dosage form.

### Epilepsy and Epilepsy Syndromes

The compositions described herein are useful in the treatment of epilepsy and epilepsy syndromes. Epilepsy is a central nervous system disorder in which nerve cell activity in the brain becomes disrupted, causing recurrent seizures that can manifest as abnormal movements, periods of unusual behavior, sensations, and sometimes loss of consciousness. Seizure symptoms will vary widely, from a simple blank stare for a few seconds to repeated twitching of the arms or legs during a seizure.

Epilepsy may involve a generalized seizure, involving multiple areas of the brain, or a partial or focal seizure. All areas of the brain are involved in a generalized seizure. A person experiencing a generalized seizure may cry out or make some sound, stiffen for several seconds to a minute and then have rhythmic movements of the arms and legs. The eyes may be open, and/or the person may appear not to be breathing and turn blue. The return to consciousness may be gradual, and the person may be confused from minutes to hours. The following are the main types of generalized seizures: tonic-clonic, tonic, clonic, myoclonic, myoclonic-tonic-clonic, myoclonic-atonic, atonic, and absence (typical, atypical, myoclonic, eyelid myoclonia) seizures, and epileptic spasms. In a partial or focal seizure, only part of the brain is involved, so only part of the body is affected. Depending on the part of the brain having abnormal electrical activity, symptoms may vary.

Epilepsy, as described herein, includes a generalized, partial, complex partial (e.g., seizures involving only part of the brain, but where consciousness is compromised), tonic clonic, clonic, tonic, refractory seizures, status epilepticus, absence seizures, febrile seizures, or temporal lobe epilepsy.

The compositions described herein may also be useful in the treatment of epilepsy syndromes. Severe syndromes with diffuse brain dysfunction caused, at least partly, by some aspect of epilepsy, are also referred to as epileptic encephalopathies. These are associated with frequent seizures that are resistant to treatment and severe cognitive dysfunction, for instance West syndrome.

In some embodiments, the epilepsy syndrome comprises epileptic encephalopathy, Dravet syndrome, Angelman syndrome, CDKL5 disorder, frontal lobe epilepsy, infantile spasms, West's syndrome, Juvenile Myoclonic Epilepsy, Landau-Kleffner syndrome, Lennox-Gastaut syndrome, Ohtahara syndrome, PCDH19 epilepsy, or Glut1 deficiency. In some embodiments, the epilepsy syndrome is childhood absence epilepsy (CAE). In some embodiments, the epilepsy syndrome is juvenile absence epilepsy (JAE). In some embodiments, the epilepsy syndrome is Lennox-Gastaut syndrome. In some embodiments, the epilepsy syndrome is SLC6A1 epileptic encephalopathy. In some embodiments, the epilepsy syndrome is associated with mutations in the genes that code for T-type calcium channels (e.g., CACNA1G, EEF1A2, and GABRG2 for genetic generalized epilepsy (GGE) and LGI1, TRIM3, and GABRG2 for non-acquired focal epilepsy (NAFE)), as discussed, for example, in Feng, Y C A, et al., “Ultra-Rare Genetic Variation in the Epilepsies: A Whole-Exome Sequencing Study of 17,606 Individuals,” Am. J. Human Gen. 2019; 105(2):267-282. In some embodiments, the epilepsy syndrome is Doose syndrome or myoclonic astatic epilepsy. In some embodiments, the epilepsy syndrome is epileptic encephalopathy with continuous spike and wave during sleep (CSWS). In some embodiments, the epilepsy syndrome is Landau Kleffner Syndrome (LKS). In some embodiments, the epilepsy syndrome is Jeavons syndrome.

### Absence Seizures

Absence seizures are one of the most common seizure types in patients with idiopathic generalised epilepsy (IGE) (Berg et al., Epilepsia 2000). Absence seizures are relatively brief, non-convulsive seizures characterised by abrupt onset of loss of awareness and responsiveness, usually lasting between 10-30 seconds in duration, with a rapid return to normal consciousness without post-ictal confusion. The seizures are characterised on an accompanying EEG recording by the abrupt onset and offset of generalised 1-6 Hz (e.g., 3 Hz) spike and wave discharges. Absence seizure often occur multiple times per day, interrupt learning and psychosocial functioning, and present a risk of injury because of the frequent episodes of loss of awareness. Typically, absence seizures begin in early childhood and remit by teenage years. However, in a minority of patients they persist into adulthood where they are often drug resistant and may be accompanied by other seizure types such as generalised tonic-clonic seizures. In these adult patients, the absence seizures are usually highly disabling, in particular by disqualifying the sufferer from obtaining a motor vehicle license or pursuing occupations and hobbies in which the seizures-associated periods of loss of awareness pose a safety risk, and are associated with significant psychosocial disabilities (Wirrell et al., 1997).

While there is a common perception that absence seizures are relatively “easy” to treat, a randomised control trial in patients with childhood absence epilepsy showed that even the most effective anti-epileptic drugs, ethosuximide and valproate, only completely controlled the seizures in 53% and 58% of patients, respectively, at 16 weeks as assessed by video-EEG recordings (Glauser et al., 2010), and 45% and 44% respectively at 12 months (Glauser et al., 2013). Lamotrigine, another antiepileptic drug commonly used to treat absence seizures, only controlled the seizures in 29% of patients at 16 weeks, and 21% of patients at 12 months. Furthermore, both ethosuximide and valproate are commonly associated with intolerable side effects (occurring in 24% of patients treated with either of these drugs) (Glauser et al., 2010), and the latter is now generally considered to be contraindicated in girls and women of childbearing potential. Other treatment options for absence seizures are limited, with only benzodiazepines having established efficacy, and these are commonly poorly tolerated due to sedative and cognitive side effects. Absence seizures persisting into adult life are particularly difficult to treat, with patients often being treated with multiple drugs resulting in significant side-effects without attaining seizure control.

There is abundant evidence that low threshold (T-type) calcium channels play a role in the generation and maintenance of absence seizures, being a key component of the oscillatory burst firing that occurs in thalamocortical neurones during absence seizures (Pinault and O'Brien, 1997). In some embodiments, the present disclosure is directed towards a method for treating absence seizures with a composition comprising a compound of Formula (I) or Formula (IA) or a pharmaceutically acceptable salt thereof, as described herein. In some embodiments, the absence seizures are refractory absence seizures. In some embodiments, the absence seizures are refractory to an anti-epileptic drug (e.g., ethosuximide, valproic acid, or lamotrigine).

In some embodiments, the subject has epilepsy. In some embodiments, the absence seizures are atypical absence seizures. In some embodiments, the absence seizures comprise adult absence seizures, juvenile absence seizures, or childhood absence seizures.

In some embodiments, the methods described herein further comprise identifying a subject having absence seizures.

### Genetic Epilepsies

In some embodiments, the epilepsy or epilepsy syndrome is a genetic epilepsy or a genetic epilepsy syndrome. In some embodiments, the epilepsy or epilepsy syndrome is genetic generalized epilepsy. In some embodiments, epilepsy or an epilepsy syndrome comprises epileptic encephalopathy, epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, KCNQ2 epileptic encephalopathy, and KCNT1 epileptic encephalopathy.

In some embodiments, the methods described herein further comprise identifying a subject having epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, KCNQ2 epileptic encephalopathy, and KCNT1 epileptic encephalopathy) prior to administration of a composition described herein.

In one aspect, disclosed herein is a method of treating epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, KCNQ2 epileptic encephalopathy, and KCNT1 epileptic encephalopathy) comprising administering to a subject in need thereof a composition described herein.

A composition of the present invention may also be used to treat an epileptic encephalopathy, wherein the subject has a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, and WWOX.

In some embodiments, the methods described herein further comprise identifying a subject having a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, WWOX, CACNA1G, CACNA1H, and CACNA1I prior to administration of a composition described herein.

A composition of the present invention may also be used to treat an epileptic encephalopathy, wherein the subject has a mutation in one or more of ADSL, ALDH5A1, ALDH7A1, ALG13, ARG1, ARHGEF9, ARX, ATP1A2, ATP1A3, ATRX, BRAT1, C12orf57, CACNA1A, CACNA2D2, CARS2, CASK, CDKL5, CHD2, CHRNA2, CHRNA4, CHRNB2, CLCN4, CLN2 (TPP1), CLN3, CLN5, CLN6, CLN8, CNTNAP2, CSTB, CTSD, DDC, DEPDC5, DNAJC5, DNM1, DOCK7, m DYRK1A, EEF1A2, EFHC1, EHMT1, EPM2A, FARS2, FOLR1, FOXG1, FRRS1L, GABBR2, GABRA1, GABRB2, GABRB3, GABRG2, GAMT, GATM, GLRA1, GNAO1, GOSR2, GRIN1, GRIN2A, GRIN2B, HCN1, HNRNPU, IER3IP1, IQSEC2, ITPA, JMJD1C, KANSL1, KCNA2, KCNB1, KCNC1, KCNH2, KCNJ10, KCNMA1, KCNQ2, KCNQ3, KCNT1, KCTD7, LGI1, LIAS, MBD5, MECP2, MEF2C, MFSD8, MOCS1, MOCS2, MTOR, NEDD4L, NEXMIF, NGLY1, NHLRC1, NPRL3, NRXN1, PACS1, PCDH19, PIGA, PIGN, PIGO, PLCB1, PNKD, PNKP, PNPO, POLG, PPT1, PRICKLE1, PRIMA1, PRRT2, PURA, QARS, RELN, ROGDI, SATB2, SCARB2, SCN1A, SCN1B, SCN2A, SCN3A, SCN8A, SCN9A, SERPINI1, SGCE, SIK1, SLC12A5, SLC13A5, SLC19A3, SLC25A12, SLC25A22, SLC2A1, SLC35A2, SLC6A1, SLC6A8, SLC9A6, SMC1A, SNX27, SPATA5, SPTAN1, ST3GAL5, STRADA, STX1B, STXBP1, SUOX, SYN1, SYNGAP1, SYNJ1, SZT2, TBCID24, TCF4, TPK1, TSC1, TSC2, UBE3A, WDR45, WWOX, ZDHHC9, ZEB2, ABAT, ARHGEF15, ATP6AP2, CACNA1H, CACNB4, CASR, CERS1, CNTN2, CPA6, DIAPH1, FASN, GABRD, GAL, GPHN, KCNA1, KCND2, KCNH5, KPNA7, LMNB2, NECAP1, PIGG, PIGQ, PIK3AP1, PRDM8, PRICKLE2, RBFOX1, RBFOX3, RYR3, SCN5A, SETD2, SLC35A3, SNAP25, SRPX2, ST3GAL3, TBLIXR1, AMT, GCSH, GLDC, FLNA, PTEN, and RANBP2.

In some embodiments, the methods described herein further comprise identifying a subject having a mutation in one or more of ADSL, ALDH5A1, ALDH7A1, ALG13, ARG1, ARHGEF9, ARX, ATP1A2, ATP1A3, ATRX, BRAT1, C12orf57, CACNA1A, CACNA2D2, CARS2, CASK, CDKL5, CHD2, CHRNA2, CHRNA4, CHRNB2, CLCN4, CLN2 (TPP1), CLN3, CLN5, CLN6, CLN8, CNTNAP2, CSTB, CTSD, DDC, DEPDC5, DNAJC5, DNM1, DOCK7, DYRK1A, EEF1A2, EFHC1, EHMT1, EPM2A, FARS2, FOLR1, FOXG1, FRRS1L, GABBR2, GABRA1, GABRB2, GABRB3, GABRG2, GAMT, GATM, GLRA1, GNAO1, GOSR2, GRIN1, GRIN2A, GRIN2B, HCN1, HNRNPU, IER3IP1, IQSEC2, ITPA, JMJD1C, KANSL1, KCNA2, KCNB1, KCNC1, KCNH2, KCNJ10, KCNMA1, KCNQ2, KCNQ3, KCNT1, KCTD7, LGI1, LIAS, MBD5, MECP2, MEF2C, MFSD8, MOCS1, MOCS2, MTOR, NEDD4L, NEXMIF, NGLY1, NHLRC1, NPRL3, NRXN1, PACS1, PCDH19, PIGA, PIGN, PIGO, PLCB1, PNKD, PNKP, PNPO, POLG, PPT1, PRICKLE1, PRIMA1, PRRT2, PURA, QARS, RELN, ROGDI, SATB2, SCARB2, SCN1A, SCN1B, SCN2A, SCN3A, SCN8A, SCN9A, SERPINI1, SGCE, SIK1, SLC12A5, SLC13A5, SLC19A3, SLC25A12, SLC25A22, SLC2A1, SLC35A2, SLC6A1, SLC6A8, SLC9A6, SMC1A, SNX27, SPATA5, SPTAN1, ST3GAL5, STRADA, STX1B, STXBP1, SUOX, SYN1, SYNGAP1, SYNJ1, SZT2, TBC1D24, TCF4, TPK1, TSC1, TSC2, UBE3A, WDR45, WWOX, ZDHHC9, ZEB2, ABAT, ARHGEF15, ATP6AP2, CACNA1H, CACNB4, CASR, CERS1, CNTN2, CPA6, DIAPH1, FASN, GABRD, GAL, GPHN, KCNA1, KCND2, KCNH5, KPNA7, LMNB2, NECAP1, PIGG, PIGQ, PIK3AP1, PRDM8, PRICKLE2, RBFOX1, RBFOX3, RYR3, SCN5A, SETD2, SLC35A3, SNAP25, SRPX2, ST3GAL3, TBLIXR1, AMT, GCSH, GLDC, FLNA, PTEN, and RANBP2.

A composition as disclosed herein may also be used to treat an epileptic encephalopathy, wherein the subject has a mutation in one or more of ADSL, ALDH5A1, ALDH7A1, ALG13, ARHGEF9, ARX, ASNS, ATP1A2, ATP1A3, ATP6AP2, ATRX, BRAT1, CACNA1A, CASK, CDKL5, CHD2, CHRNA2, CHRNA4, CHRNA7, CHRNB2, CLCN4, CLN3, CLN5, CLN6, CLN8, CNTNAP2, CSTB, CTNNB1, CTSD (CLN10), CTSF, DDX3X, DEPDC5, DNAJC5 (CLN4B), DNM1, DYRK1A, EEF1A2, EHMT1, EPM2A, FLNA, FOLR1, FOXG1, FRRS1L, GABBR2, GABRA1, GABRB2, GABRB3, GABRG2, GAMT, GATM, GLDC, GNAO1, GOSR2, GRIN1, GRIN2A, GRIN2B, HNRNPU, IQSEC2, KANSL1, KCNA2, KCNB1, KCNC1, KCNH1, KCNJ10, KCNMA1, KCNQ2, KCNQ3, KCNT1, KCTD7 (CLN14), KDM6A, KIAA2022, LGI1, MAGI2, MBD5, MECP2, MEF2C, MFSD8 (CLN7), NALCN, NGLY1, NHLRC1 (EPM2B), NPRL3, NR2F1, NRXN1, PACS1, PCDH19, PIGA PIGO, PIGV, PLCB1, PNKP, PNPO, POLG, PPP2R5D, PPT1 (CLN1), PRRT2, PURA, QARS, SATB2, SCARB2, SCN1A, SCN1B, SCN2A, SCN8A, SLC13A5, SLC19A3, SLC25A22, SLC2A1, SLC6A1, SLC6A8, SLC9A6, SMC1A, SPATA5, SPTAN1, STX1B, STXBP1, SYNGAP1, SZT2, TBC1D24, TBLIXR1, TCF4, TPP1 (CLN2), TSC1, TSC2, UBE3A, WDR45, WWOX, and ZEB2.

In some embodiments, the methods described herein further comprise identifying a subject having a mutation in one or more of ADSL, ALDH5A1, ALDH7A1, ALG13, ARHGEF9, ARX, ASNS, ATP1A2, ATP1A3, ATP6AP2, ATRX, BRAT1, CACNA1A, CASK, CDKL5, CHD2, CHRNA2, CHRNA4, CHRNA7, CHRNB2, CLCN4, CLN3, CLN5, CLN6, CLN8, CNTNAP2, CSTB, CTNNB1, CTSD (CLN10), CTSF, DDX3X, DEPDC5, DNAJC5 (CLN4B), DNM1, DYRK1A, EEF1A2, EHMT1, EPM2A, FLNA, FOLR1, FOXG1, FRRS1L, GABBR2, GABRA1, GABRB2, GABRB3, GABRG2, GAMT, GATM, GLDC, GNAO1, GOSR2, GRIN1, GRIN2A, GRIN2B, HNRNPU, IQSEC2, KANSL1, KCNA2, KCNB1, KCNC1, KCNH1, KCNJ10, KCNMA1, KCNQ2, KCNQ3, KCNT1, KCTD7 (CLN14), KDM6A, KIAA2022, LGI1, MAGI2, MBD5, MECP2, MEF2C, MFSD8 (CLN7), NALCN, NGLY1, NHLRC1 (EPM2B), NPRL3. NR2F1, NRXN1, PACS1, PCDH19, PIGA PIGO, PIGV, PLCB1, PNKP, PNPO, POLG, PPP2R5D, PPT1 (CLN1), PRRT2, PURA, QARS, SATB2, SCARB2, SCN1A, SCN1B, SCN2A, SCN8A, SLC13A5, SLC19A3, SLC25A22, SLC2A1, SLC6A1, SLC6A8, SLC9A6, SMC1A, SPATA5, SPTAN1, STX1B, STXBP1, SYNGAP1, SZT2, TBC1D24, TBLIXR1, TCF4, TPP1 (CLN2), TSC1, TSC2, UBE3A, WDR45, WWOX, and ZEB2.

A composition as disclosed herein may also be used to treat an epileptic encephalopathy, wherein the subject has a mutation in one or more of ALDH7A1, ARHGEF9, ARX, ATP13A2, ATP1A2, CACNA1A, CASK, CDKL5, CHD2, CHRNA2, CHRNA4, CHRNB2, CLN3, CLN5, CLN6, CLN8, CNTNAP2, CRH, CSTB, CTSD, CTSF, DCX, DEPDC5, DNAJC5, DNM1, DYNC1H1, DYRK1A, EEF1A2, EPM2A, FLNA, FOLR1, FOXG1, GABRA1, GABRB3, GABRG2, GAMT, GATM, GNAO1, GOSR2, GRIN1, GRIN2A, GRIN2B, GRN, HCN1, HNRNPU, IQSEC2, KCNA2, KCNC1, KCNJ10, KCNQ2, KCNQ3, KCNT1, KCTD7, KIAA2022, LGI1, MECP2, MEF2C, MFSD8, NHLRC1, NRXN1, PCDH19, PIGA, PLCB1, PNKP, PNPO, POLG, PPT1, PRICKLE1, PRRT2, PURA, SCARB2, SCN1A, SCN1B, SCN2A, SCN8A, SIK1, SLC13A5, SLC25A22, SLC2A1, SLC35A2, SLC6A1, SLC9A6, SMC1A, SNAP25, SPTAN1, ST3GAL3, STX1B, STXBP1, SYN1, SYNGAP1, SZT2, TBC1D24, TBLIXR1, TCF4, TPP1, TSC1, TSC2, UBE3A, WDR45, and ZEB2.

In some embodiments, the methods described herein further comprise identifying a subject having a mutation in one or more of ALDH7A1, ARHGEF9, ARX, ATP13A2, ATP1A2, CACNA1A, CASK, CDKL5, CHD2, CHRNA2, CHRNA4, CHRNB2, CLN3, CLN5, CLN6, CLN8, CNTNAP2, CRH, CSTB, CTSD, CTSF, DCX, DEPDC5, DNAJC5, DNM1, DYNC1H1, DYRK1A, EEF1A2, EPM2A, FLNA, FOLR1, FOXG1, GABRA1, GABRB3, GABRG2, GAMT, GATM, GNAO1, GOSR2, GRIN1, GRIN2A, GRIN2B, GRN, HCN1, HNRNPU, IQSEC2, KCNA2, KCNC1, KCNJ10, KCNQ2, KCNQ3, KCNT1, KCTD7, KIAA2022, LGI1, MECP2, MEF2C, MFSD8, NHLRC1, NRXN1, PCDH19, PIGA, PLCB1, PNKP, PNPO, POLG, PPT1, PRICKLE1, PRRT2, PURA, SCARB2, SCN1A, SCN1B, SCN2A, SCN8A, SIK1, SLC13A5, SLC25A22, SLC2A1, SLC35A2, SLC6A1, SLC9A6, SMC1A, SNAP25, SPTAN1, ST3GAL3, STX1B, STXBP1, SYN1, SYNGAP1, SZT2, TBC1D24, TBLIXR1, TCF4, TPP1, TSC1, TSC2, UBE3A, WDR45, and ZEB2.

### Mood Disorders

Also provided herein are methods of using the compounds disclosed herein for treating a psychiatric disorder such as a mood disorder, for example clinical depression, postnatal depression or postpartum depression, perinatal depression, atypical depression, melancholic depression, psychotic major depression, catatonic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior. In some embodiments, the method described herein provides therapeutic effect to a subject suffering from depression (e.g., moderate or severe depression). In some embodiments, the mood disorder is associated with a disease or disorder described herein (e.g., neuroendocrine diseases and disorders, neurodegenerative diseases and disorders (e.g., epilepsy), movement disorders, tremor (e.g., Parkinson's Disease), women's health disorders or conditions).

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Peripartum depression refers to depression in pregnancy. Symptoms include irritability, crying, feeling restless, trouble sleeping, extreme exhaustion (emotional and/or physical), changes in appetite, difficulty focusing, increased anxiety and/or worry, disconnected feeling from baby and/or fetus, and losing interest in formerly pleasurable activities.

Postnatal depression (PND) is also referred to as postpartum depression (PPD) and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression, or a symptom of depression, during pregnancy. This depression is referred to herein as perinatal depression. In an embodiment, a subject experiencing perinatal depression is at increased risk of experiencing PND.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). During periods of mania the individual may feel or act abnormally happy, energetic, or irritable. They often make poorly thought out decisions with little regard to the consequences. The need for sleep is usually reduced. During periods of depression there may be crying, poor eye contact with others, and a negative outlook on life. The risk of suicide among those with the disorder is high at greater than 6% over 20 years, while self-harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, or chronic stress.

Treatment-resistant depression refers to a condition where the individuals have been treated for depression, but the symptoms do not improve. For example, antidepressants or psychological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to at least one standard pharmacological treatment, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Post-surgical depression refers to feelings of depression that follow a surgical procedure (e.g., as a result of having to confront one's mortality). For example, individuals may feel sadness or empty mood persistently, a loss of pleasure or interest in hobbies and activities normally enjoyed, or a persistent feeling of worthlessness or hopelessness.

Mood disorder associated with conditions or disorders of women's health refers to mood disorders (e.g., depression) associated with (e.g., resulting from) a condition or disorder of women's health (e.g., as described herein).

Suicidality, suicidal ideation, and suicidal behavior refer to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, and/or incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, and saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case to case basis. Symptoms of depression, and relief of the same, may be ascertained by a physician or psychologist (e.g., by a mental state examination).

In some embodiments, the mood disorder is selected from depression, major depressive disorder, bipolar disorder, dysthymic disorder, anxiety disorders, stress, post-traumatic stress disorder, bipolar disorder, and compulsive disorders. In some embodiments, the mood disorder is major depressive disorder.

In some embodiments, the method comprises monitoring a subject with a known depression scale, e.g., the Hamilton Depression (HAM-D) scale, the Clinical Global Impression-Improvement Scale (CGI), and the Montgomery-Asberg Depression Rating Scale (MADRS). In some embodiments, a therapeutic effect can be determined by reduction in Hamilton Depression (HAM-D) total score exhibited by the subject. The therapeutic effect can be assessed across a specified treatment period. For example, the therapeutic effect can be determined by a decrease from baseline in HAM-D total score after administering a composition described herein (e.g., 12, 24, or 48 hours after administration; or 24, 48, 72, or 96 hours or more; or 1 day, 2 days, 14 days, 21 days, or 28 days; or 1 week, 2 weeks, 3 weeks, or 4 weeks; or 1 month, 2 months, 6 months, or 10 months; or 1 year, 2 years, or for life).

In some embodiments, the subject has a mild depressive disorder, e.g., mild major depressive disorder. In some embodiments, the subject has a moderate depressive disorder, e.g., moderate major depressive disorder. In some embodiments, the subject has a severe depressive disorder, e.g., severe major depressive disorder. In some embodiments, the subject has a very severe depressive disorder, e.g., very severe major depressive disorder. In some embodiments, the baseline HAM-D total score of the subject (i.e., prior to treatment with a composition described herein), is at least 24. In some embodiments, the baseline HAM-D total score of the subject is at least 18. In some embodiments, the baseline HAM-D total score of the subject is between and including 14 and 18. In some embodiments, the baseline HAM-D total score of the subject is between and including 19 and 22. In some embodiments, the HAM-D total score of the subject before treatment with a composition described herein is greater than or equal to 23. In some embodiments, the baseline score is at least 10, 15, or 20. In some embodiments, the HAM-D total score of the subject after treatment with a compound or composition disclosed herein is about 0 to 10 (e.g., less than 10; 0 to 10, 0 to 6, 0 to 4, 0 to 3, 0 to 2, or 1.8). In some embodiments, the HAM-D total score after treatment with a compound or composition disclosed herein is less than 10, 7, 5, or 3. In some embodiments, the decrease in HAM-D total score is from a baseline score of about 20 to 30 (e.g., 22 to 28, 23 to 27, 24 to 27, 25 to 27, 26 to 27) to a HAM-D total score at about 0 to 10 (e.g., less than 10; 0 to 10, 0 to 6, 0 to 4, 0 to 3, 0 to 2, or 1.8) after treatment with a compound or composition disclosed herein. In some embodiments, the decrease in the baseline HAM-D total score to HAM-D total score after treatment with a compound or composition disclosed herein is at least 1, 2, 3, 4, 5, 7, 10, 25, 40, or 50). In some embodiments, the percentage decrease in the baseline HAM-D total score to HAM-D total score after treatment with a compound or composition disclosed herein is at least 50% (e.g., 60%, 70%, 80%, or 90%). In some embodiments, the therapeutic effect is measured as a decrease in the HAM-D total score after treatment with a compound or composition disclosed herein relative to the baseline HAM-D total score.

In some embodiments, the method of treating a depressive disorder, e.g., major depressive disorder, provides a therapeutic effect (e.g., as measured by reduction in the HAM-D score) within 14, 10, 4, 3, 2, or 1 days, or 24, 20, 16, 12, 10, or 8 hours or less. In some embodiments, the method of treating the depressive disorder, e.g., major depressive disorder, provides a therapeutic effect (e.g., as determined by a statistically significant reduction in HAM-D total score) within the first or second day of the treatment with a composition described herein. In some embodiments, the method of treating the depressive disorder, e.g., major depressive disorder, provides a therapeutic effect (e.g., as determined by a statistically significant reduction in HAM-D total score) within less than or equal to 14 days since the beginning of the treatment with a composition described herein. In some embodiments, the method of treating the depressive disorder, e.g., major depressive disorder, provides a therapeutic effect (e.g., as determined by a statistically significant reduction in HAM-D total score) within less than or equal to 21 days since the beginning of the treatment with a composition described herein. In some embodiments, the method of treating the depressive disorder, e.g., major depressive disorder, provides a therapeutic effect (e.g., as determined by a statistically significant reduction in HAM-D total score) within less than or equal to 28 days since the beginning of the treatment with a compound or composition disclosed herein. In some embodiments, the therapeutic effect is a decrease from baseline in HAM-D total score after treatment with a compound or composition disclosed herein. In some embodiments, the HAM-D total score of the subject before treatment with a compound or composition disclosed herein is at least 24. In some embodiments, the HAM-D total score of the subject before treatment with a compound or composition disclosed herein is at least 18. In some embodiments, the HAM-D total score of the subject before treatment with a compound or composition disclosed herein is between and including 14 and 18. In some embodiments, the decrease in HAM-D total score after treating the subject with a compound or composition disclosed herein relative to the baseline HAM-D total score is at least 10. In some embodiments, the decrease in HAM-D total score after treating the subject with a compound or composition disclosed herein relative to the baseline HAM-D total score is at least 15. In some embodiments, the HAM-D total score associated with treating the subject with a compound or composition disclosed herein is no more than a number ranging from 6 to 8. In some embodiments, the HAM-D total score associated with treating the subject with a compound or composition disclosed herein is no more than 7.

In some embodiments, the method provides therapeutic effect (e.g., as measured by reduction in Clinical Global Impression-Improvement Scale (CGI)) within 14, 10, 4, 3, 2, or 1 days, or 24, 20, 16, 12, 10, or 8 hours or less. In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, the therapeutic effect is a decrease from baseline in CGI score at the end of a treatment period (e.g., 14 days after administration).

A therapeutic effect for major depressive disorder can be determined by a reduction in Montgomery-Asberg Depression Rating Scale (MADRS) score exhibited by the subject. For example, the MADRS score can be reduced within 4, 3, 2, or 1 days; or 96, 84, 72, 60, 48, 24, 20, 16, 12, 10, 8 hours or less. The MADRS is a ten-item diagnostic questionnaire (regarding apparent sadness, reported sadness, inner tension, reduced sleep, reduced appetite, concentration difficulties, lassitude, inability to feel, pessimistic thoughts, and suicidal thoughts) that psychiatrists use to measure the severity of depressive episodes in patients with mood disorders. In some embodiments, the therapeutic effect is a decrease from baseline in MADRS score at the end of a treatment period (e.g., 14 days after administration).

### Pain

The compounds and compositions described herein may be useful in the treatment of pain. In some embodiments, the pain comprises acute pain, chronic pain, neuropathic pain, inflammatory pain, nociceptive pain, central pain (e.g., thalamic pain), or migraine. In some embodiments, the pain comprises acute pain or chronic pain. In some embodiments, the pain comprises neuropathic pain, inflammatory pain, or nociceptive pain. In some embodiments, the pain comprises central pain (e.g., thalamic pain). In some embodiments, the pain comprises migraine.

In some embodiments, the methods described herein further comprise identifying a subject having pain (e.g., acute pain, chronic pain, neuropathic pain, inflammatory pain, nociceptive pain, central pain (e.g., thalamic pain), or migraine) prior to administration of a dosage form or composition described herein (e.g., a dosage form or composition including a compound of Formula (I) or Formula (IA) or a pharmaceutically acceptable salt thereof.

### Tremor

The methods described herein can be used to treat tremor, for example a compound or composition disclosed herein can be used to treat cerebellar tremor or intention tremor, dystonic tremor, essential tremor, orthostatic tremor, Parkinsonian tremor, physiological tremor, or rubral tremor. Tremor includes hereditary, degenerative, and idiopathic disorders such as Wilson's disease (hereditary), Parkinson's disease (degenerative), and essential tremor (idiopathic); metabolic diseases; peripheral neuropathies (associated with Charcot-Marie-Tooth, Roussy-Levy, diabetes mellitus, complex regional pain syndrome); toxins (nicotine, mercury, lead, carbon monoxide, manganese, arsenic, toluene); drug-induced (neuroleptics tricyclics, lithium, cocaine, alcohol, adrenaline, bronchodilators, theophylline, caffeine, steroids, valproate, amiodarone, thyroid hormones, vincristine); and psychogenic disorders. Clinical tremor can be a neuropathic tremor, and can be classified into physiologic tremor, enhanced physiologic tremor, essential tremor syndromes (including classical essential tremor), primary orthostatic tremor, task- and position-specific tremor, dystonic tremor, parkinsonian tremor, cerebellar tremor, Holmes' tremor (i.e., rubral tremor), palatal tremor, toxic or drug-induced tremor, and psychogenic tremor. The tremor may be familial tremor.

In some embodiments, the subjects are selected for treatment with a compound of Formula (IA), Formula (I) or a pharmaceutical composition of a compound of Formula (IA) or Formula (I) due to a clinical diagnosis of essential tremor. In some embodiments, the subjects selected for treatment with a compound of Formula (IA), Formula (I) or a pharmaceutical composition of a compound of Formula (IA) or Formula (I) have essential tremor, but do not have intention tremor.

Tremor is an involuntary, rhythmic oscillation of one or more body parts (e.g., hands, arms, eyes, face, head, vocal folds, trunk, and/or legs).

Cerebellar tremor or intention tremor is a slow, broad tremor of the extremities that occurs after a purposeful movement. Cerebellar tremor is caused by lesions in or damage to the cerebellum or pathways resulting from, e.g., tumor, stroke or other focal lesion disease (e.g., multiple sclerosis) or a neurodegenerative disease.

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occur irregularly and often can be relieved by complete rest or certain sensory maneuvers.

Essential tremor or benign essential tremor is the most common type of tremor. Essential tremor may be mild and nonprogressive in some, and may be slowly progressive, starting on one side of the body but typically affecting both sides. The hands are most often affected, but the head, voice, tongue, legs, and trunk may also be involved. Tremor frequency may decrease as the person ages, but severity may increase. Heightened emotion, stress, fever, physical exhaustion, or low blood sugar may trigger tremors and/or increase their severity. Symptoms generally evolve over time and can be both visible and persistent following onset.

Orthostatic tremor is characterized by fast (e.g., greater than 12 Hz) rhythmic muscle contractions that occur in the legs and trunk immediately after standing. Cramps are felt in the thighs and legs and the patient may shake uncontrollably when asked to stand in one spot. Orthostatic tremor may occur in patients with essential tremor.

Parkinsonian tremor is caused by damage to structures within the brain that control movement. Parkinsonian tremor is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Rubral tremor is characterized by coarse slow tremor which can be present at rest, at posture, and with intention. The tremor is associated with conditions that affect the red nucleus in the midbrain, such as a stroke.

In some embodiments, the tremor is selected from essential tremor, Parkinson's tremor, or Cerebellar tremor.

The efficacy of the compound or composition disclosed herein for treating essential tremor can be measured by methods known in the art, such as the methods described in the following references: Ferreira, J. J. et al., “MDS Evidence-Based Review of Treatments for Essential Tremor,” Mov. Disord. 2019 July; 34(7):950-958; Elble, R. et al., “Task Force Report: Scales for Screening and Evaluating Tremor,” Mov. Disord. 2013 November; 28(13):1793-800; Deuschl G. et al., “Treatment of patients with essential tremor,” Lancet Neurol. 2011; 10:148-61; and Reich S. G. et al., “Essential Tremor,” Med. Clin. N. Am. 2019; 103:351-356. The disclosures of the references are herein incorporated in their entirety.

In some embodiments, the methods described herein result in at least 25% reduction in the upper limb tremor score, wherein the tremor score may be converted to amplitude, as compared to a baseline. For example, in certain embodiments, the methods described herein result in about 40% mean reduction in tremor amplitude as measured by The Essential Tremor Rating Assessment Scale (TETRAS) upper limb score, described, for example, in Elble, R. J., “The Essential Tremor Rating Assessment Scale,” J. Neurol. Neuromed. 2016; 1(4):34-38. In some embodiments, the methods described herein result in at least 25% reduction in TETRAS performance score as compared to the baseline. In some embodiments, the methods described herein result in at least 35% average reduction in symptom severity as compared to the baseline, as measured by TETRAS performance score.

### Ataxia

Ataxia, including both cerebellar ataxia and spinal ataxia (e.g., posterior spinal ataxia), generally involves the loss or failure of coordination. Patients exhibiting ataxia may have difficulty regulating the force, range, direction, velocity, and rhythm involved in posture, balance, and limb movement. Ataxia of the trunk, for example, can result in increased postural sway, and an inability to maintain the center of gravity over the base of support. Ataxia and primary or secondary symptoms of ataxic gait and tremor of the limbs may be accompanied by speech disturbance, dysphagia, abnormal ventilation and speech, and involuntary eye movements, dystonia, pyramidal or extrapyramidal symptoms, thereby substantially interfering with the activities of daily life.

As noted above, ataxia may result from a wide range of underlying diseases and conditions in a patient, including cerebellar and neurodegenerative disorders and diseases resulting from chronic or long-term exposure to toxins. Symptoms of ataxia may result from a wide range of diseases, disorders, and environmental factors, including infectious diseases, metabolic diseases, neurodegenerative diseases, genetic diseases, vascular diseases, neoplastic diseases, demyelinating diseases, neuromuscular diseases, and diseases resulting from long-term or chronic exposure to toxins (including drugs and alcohol), among a variety of others; in one embodiment, for example, the ataxia is the result of a metabolic disease, a neurodegenerative disease, a vascular disease, a neuromuscular disease, or a disease resulting from long-term or chronic exposure to toxins. Diseases, disorders, syndromes, and conditions that may result in ataxic symptoms that may be treated according to the methods described herein include, but are not limited to, amyotrophic lateral sclerosis, benign paroxysmal positional vertigo, cerebellar ataxia type 1 (autosomal recessive), cerebellar ataxias (autosomal recessive), cerebellar ataxias (dominant pure), cerebellar cortical atrophy, cerebellar degeneration (subacute), cerebellar dysfunction, cerebellar hypoplasia, cerebellar hypoplasia (endosteal sclerosis), cerebellar hypoplasia (tapetoretinal degeneration), cerebelloparenchymal autosomal recessive disorder 3, cerebelloparenchymal disorder V, cerebellum agenesis (hydrocephaly), cerebral amyloid angiopathy (familial), cerebral palsy, demyelinating disorder, dorsal column conditions, dysautonomia, dysequilibrium syndrome, dysethesis, endocrine diseases, diseases caused by chronic exposure to toxins (e.g., alcohol, drugs, antiepileptics, neuroleptics), Fragile X/Tremor ataxia syndrome, Friedreich's ataxia, frontal lobe dysfunction, genetic diseases, granulomatous angiitis of the central nervous system, Hallervorden-Spatz disease, hereditary motor and sensory neuropathy, hydrocephalus (e.g., low or normal pressure), hypotonia, congenital nystagmus, ataxia and abnormal auditory brainstem response, infantile onset spinocerebellar ataxia, Machado-Joseph disease, Meniere's disease, metabolic disorders, Miller Fisher Syndrome, Minamata disease, multiple sclerosis, muscular dystrophy, Myoclonus-ataxia, neurodegenerative diseases, olivopontocerebellar atrophy, paraneoplastic disorders, parkinsonism (atypical), peroneal muscular atrophy, phenyloin toxicity, posterior column ataxia with retinitis pigmentosa, post-polio syndrome, severe damage to the brain (caused by, e.g., head injury, brain surgery, multiple sclerosis or cerebral palsy, chronic alcohol/drug abuse, chronic exposure to toxins, viral infections, or brain tumor), spastic hemiparesis, spastic paraplegia 23, spastic paraplegia glaucoma precocious puberty, SPG, spinocerebellar ataxia, spinocerebellar ataxia (amyotrophy-deafness), spinocerebellar ataxia (dysmorphism), spinocerebellar ataxia 11, spinocerebellar ataxia 17, spinocerebellar ataxia 20, spinocerebellar ataxia 25, spinocerebellar ataxia 29, spinocerebellar ataxia 42, spinocerebellar ataxia 3, spinocerebellar ataxia (autosomal recessive 1), spinocerebellar ataxia (autosomal recessive 3), spinocerebellar ataxia (autosomal recessive 4), spinocerebellar ataxia (autosomal recessive 5), spinocerebellar ataxia (autosomal recessive, with axonal neuropathy), spinocerebellar ataxia (Machado-Joseph type II), spinocerebellar ataxia (X-linked, 2), spinocerebellar ataxia (X-linked, 3), spinocerebellar ataxia (X-linked, 4), spinocerebellar degenerescence (book type), stroke (e.g., acute or hemorrhagic), vertebral artery dissection, vertebral-basilar insufficiency, and diseases caused by vitamin deficiencies, among a variety of others. In one embodiment, the ataxia is the result of a disease selected from Spinocerebellar ataxia, Friedriech's ataxia, and fragile X/tremor ataxia syndrome. In another particular embodiment, the ataxia is the result of Spinocerebellar ataxia or Fragile X/tremor ataxia syndrome.

### Tinnitus

Methods of treating tinnitus in a subject in need thereof are provided herein and comprise administering a compound or composition as disclosed herein. Tinnitus is a condition in which those affected perceive sound in one or both ears or in the head when no external sound is present. Often referred to as “ringing” in the ears, tinnitus can occur intermittently or consistently with a perceived volume ranging from low to painfully high. However, the perceived volume of tinnitus can vary from patient to patient where an objective measure of tinnitus volume in one patient may be perceived as painful while, in another patient, the same volume may be perceived as subtle.

### Sleep Disorders

Methods of treating or preventing sleep disorder (e.g., narcolepsy) comprising administering a compound or composition disclosed herein are provided herein. For example, a sleep disorder may be a central disorder of hypersomnolence, narcolepsy type I, narcolepsy type II, idiopathic hypersomnia, Kleine-Levin syndrome, hypersomnia due to a medical disorder, hypersomnia due to a medication or substance, hypersomnia associated with a psychiatric disorder, insufficient sleep syndrome, circadian rhythm sleep-wake disorders, delayed sleep-wake phase disorder, advanced sleep-wake phase disorder, irregular sleep-wake rhythm, non-24-hour sleep-wake rhythm disorder, shift work disorder, jet lag disorder, or circadian rhythm sleep-wake disorder not otherwise specified (NOS).

### Combination Therapy

In another aspect, a compound or composition described herein (e.g., for use in modulating a T-type calcium ion channel) may be administered in combination with at least one other agent or therapy. A subject to be administered a compound or composition disclosed herein may have a disease, disorder, or condition, or at least one symptom thereof, that would benefit from treatment with another agent or therapy. In certain embodiments, these diseases or conditions can relate to epilepsy or an epilepsy syndrome (e.g., absence seizures, juvenile myoclonic epilepsy, or a genetic epilepsy) or tremor (e.g., essential tremor).

### Antiepilepsy Agents

Anti-epilepsy agents include brivaracetam, carbamazepine, clobazam, clonazepam, diazepam, divalproex, eslicarbazepine, ethosuximide, ezogabine, felbamate, gabapentin, lacosamide, lamotrigine, levetiracetam, lorazepam, oxcarbazepine, midazolam, permpanel, phenobarbital, phenytoin, pregabalin, primidone, rufinamide, tigabine, topiramate, valproic acid, vigabatrin, and zonisamide.

### Analgesics

Analgesics are therapeutic agents that are used to relieve pain. Examples of analgesics include opiates and morphinomimetics, such as fentanyl and morphine; paracetamol; NSAIDs, and COX-2 inhibitors. Given the ability of the compounds disclosed herein to treat pain via inhibition of T-type calcium channels (e.g., Cav3.1, Cav3.2, and Cav3.3), combination with analgesics are particularly envisioned.

### Tremor Medications

Tremor medications include propranolol, primidone, clonazepam, diazepam, lorazepam, alprazolam, gabapentin, topiramate, midazolam, atenolol, klonopin, alprazolam, nebivolol, carbidopa/levodopa, clonazepam, hydrochlorothiazide/metoprolol, gabapentin enacarbil, labetalol, lactulose, lamotrigine, metoprolol, nadolol, hydrochlorothiazide, and zonisamide.

The present disclosure will be more fully understood by reference to the following Examples.

### Examples

In order that the embodiments described herein may be more fully understood, the following examples are set forth. The synthetic and biological examples described in this application are offered to illustrate the compounds, pharmaceutical compositions, and methods provided herein and are not to be construed in any way as limiting their scope.

In the Examples that follow, purification by chromatography refers to purification using the Biotage® Isolera™ One purification system. Where products were purified using a Santai® SepaFlash™ Irregular Silica, “Santai SepaFlash™ Irregular Silica” refers to a pre-packed polypropylene column containing unbonded activated silica with irregular particles with average size of 40-63 μm and nominal 60 Å porosity. Fractions containing the required product (identified by TLC and/or LCMS analysis) were pooled, the organic fraction recovered by evaporation, to give the final product. Where thin layer chromatography (TLC) has been used, it refers to silica-gel TLC plates, typically 3×6 cm silica-gel on glass plates with a fluorescent indicator (254 nm), (e.g., UV—III). Microwave experiments were carried out using a Biotage Initiator+, which uses a single-mode resonator and dynamic field tuning. Temperatures from 40-300° C. can be achieved, and pressures of up to 30 bar can be reached.

NMR spectra were obtained on a Bruker Ascend™ 400 MHz, 5 mm BBFO probe H, C, F, P, single Z gradient, two channel instrument running TopSpin 4.1.

Compound names were standardly generated using the Structure to Name function in ChemDraw Professional 20.

### Abbreviations:

CD₃OD Deuterated methanol
DBU 1,8-Diazabicyclo[5.4.0]undec-7-ene
DCM Dichloromethane
DIPEA N,N-Diisopropylethylamine
DMF N,N-Dimethylformamide
DMSO Dimethylsulfoxide
DMSO-d6 Deuterated dimcthylsulfoxide-d6
DPPA Diphenylphosphoryl azide
FA Formic acid
HATU 2-(7-Azabenzotriazol-1-yl)-N,N,N′,N′-tetramethyluronium hexafluorophosphate
K₂CO₃ Potassium carbonate
MeCN Acetonitrile
McOH Methanol
PE Petroleum ether
TFA Trifluoroacetic acid
THF Tetrahydrofuran

### Analytical LC-MS Conditions

Method 1 included the following conditions: Column: HALO C18 2.7 μm, 3.0 mm×30 mm. Mobile Phase: MeCN (0.05% HCOOH)-Water (0.05% HCOOH). Gradient: MeCN from 5% to 95% over 1.4 min, hold 0.6 min, total run time is 2.5 min. Flow rate:1.8 mL/min. Column Temp: 50° C. Wavelength: 214 and 254 nm PDA. A Shimadzu LCMS 2020 Mass Spectrometer was used.

Method 2 included the following conditions: Column: XBridge C18 3.5 μm, 2.1 mm×50 mm. Mobile Phase: MeCN-Water (0.1% NH₄OH). Gradient: MeCN from 5% to 95% over 1.8 min, hold 0.7 min, total run time is 3.0 min. Flow rate: 1.0 mL/min. Column Temp: 50° C. Wavelength: 214 and 254 nm PDA. A Shimadzu LCMS 2020 Mass Spectrometer was used.

### Analytical HPLC Conditions

Method A included the following conditions: Column: ZORBAX ECLIPES PLUS C18, 1.8 μm, 4.6 mm×50 mm. Mobile phase: Water (0.05% TFA)-MeCN (0.05% TFA). Gradient: MeCN from 5% to 95% over 4.5 mins, hold 1 min, total run time is 7.0 mins. Column Temp: 55° C. Flow rate: 2.0 mL/min. Wavelength Range: from 190 nm to 800 nm. Instrument: SHIMADZU LC-2030C 3D Plus.

Method B included the following conditions: Column: XBridge BEH C18 2.5 μm 3.0 mm×30 mm. Mobile phase: Water (0.1% NH₃/H₂O)-MeCN (100%). Gradient: MeCN from 5% to 95% over 5.5 mins, hold 1 min, total run time is 8 mins. Column Temp: 45° C. Flow rate: 1.5 mL/min. Wavelength Range: from 190 nm to 800 nm. Instrument: SHIMADZU LC-2030C 3D Plus.

Analytical HNMR Conditions: HNMR: BRUKER 400; Temp: 25° C.

### Example 1—Preparation of N-(tert-butyl)-2-(6-(2-((3,5-dichlorophenyl)amino)-2-oxoethyl)-2,6-diazaspiro[3.3]heptan-2-yl)acetamide

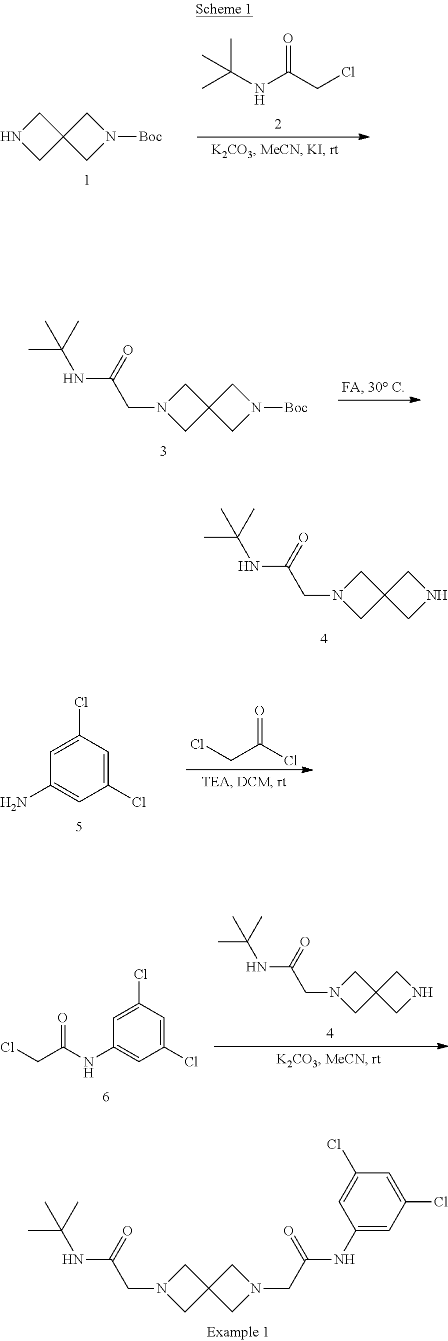

### General Method 1:

### Step 1: Preparation of tert-butyl 6-(2-(tert-butylamino)-2-oxoethyl)-2,6-diazaspiro[3.3]heptane-2-carbo late Intermediate 3

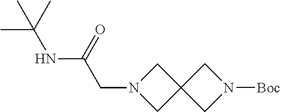 [SMILES: CC(C)(C)NC(=O)CN1CC2(C1)CN(C(=O)OC(C)(C)C)C2]

To a solution of tert-butyl 2,6-diazaspiro[3.3]heptane-2-carboxylate (2.0 g, 10 mmol) (Intermediate 3) in MeCN (20 mL) was added N-tert-butyl-2-chloro-acetamide (1.51 g, 10.mmol) (Intermediate 2), KI (1.67, 10 mmol) and K₂CO₃ (4.18 g 30 mmol). The reaction solution was stirred at room temperature overnight. The reaction was concentrated in vacuo, and the residue taken up in EtOAc (3×20 mL). The organics were washed with water (2×5 mL) and then saturated brine solution (1×5 mL). The combined organic layer was separated and dried (Na₂SO₄) before concentration in vacuo. The resultant residue was then purified by flash column chromatography eluting with 5% MeOH in DCM to provide tert-butyl 6-[2-(tert-butylamino)-2-oxo-ethyl]-2,6-diazaspiro[3.3]heptane-2-carboxylate (2.15 g, 65% yield, 95% purity) as a yellow oil. MS obsd. (ESI+): [(M+H)⁺]: 312.1 Rₜ:1.28 min (Method 2).

### Step 2: Preparation of N-(tert-butyl)-2-(2,6-diazaspiro[3.3]heptan-2-yl)acetamide (Intermediate 4)

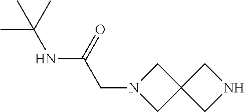

Tert-butyl 6-[2-(tert-butylamino)-2-oxo-ethyl]-2,6-diazaspiro[3.3]heptane-2-carboxylate (2.15 g, 7.0 mmol) (Intermediate 3) was added to FA (30 mL) and stirred at 30° C. for 18 hours. The reaction was concentrated in vacuo to provide N-tert-butyl-2-(2,6-diazaspiro[3.3]heptan-2-yl)acetamide (3.1 g, 100% yield, 47% purity) as a yellow oil. MS obsd. (ESI+): [(M+H)⁺]: 212.0 Rₜ: 0.70 min (Method 1).

### Step 3: Preparation of 2-chloro-N-(3,5-dichlorophenyl)acetamide (Intermediate 6)

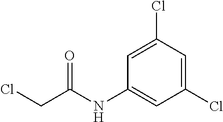 [SMILES: O=C(CCl)Nc1cc(Cl)cc(Cl)c1]

To a solution of 3,5-dichloroaniline (200 mg, 1.23 mmol) (Intermediate 5) in DCM (5 mL) was added chloroacetyl chloride (0.10 mL, 1.23 mmol) and TEA (0.51 mL, 3.7 mmol). The reaction solution was stiffed at room temperature overnight. The reaction was concentrated in vacuo, and the residue was taken up in EtOAc (3×5 mL). The organics were washed with water (2×3 mL) and then saturated brine solution (3 mL). The combined organics were separated and dried (Na₂SO₄) before concentration in vacuo. The resultant residue was purified by flash column chromatography eluting with 15% EtOAc in PE to provide 2-chloro-N-(3,5-dichlorophenyl)acetamide (208 mg, 67% yield, 95% purity) as an off-white solid.

¹H NMR (400 MHz, DMSO) δ 10.63 (s, 1H), 7.65 (d, J=2.0 Hz, 2H), 7.34 (t, J=1.8 Hz, 1H), 4.29 (s, 2H).

### Step 4: Preparation of N-(tert-butyl)-2-(6-(2-((3,5-dichlorophenyl)amino)-2-oxoethyl)-2,6-diazaspiro[3.3]heptan-2-yl)acetamide (Example 1)

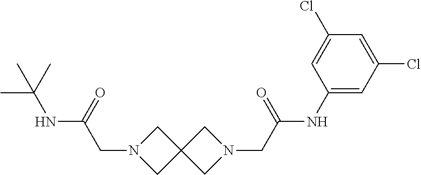 [SMILES: CC(C)(C)NC(=O)CN1CC2(CN(CC(=O)Nc3cc(Cl)cc(Cl)c3)C2)C1]

To a solution of N-tert-butyl-2-(2,6-diazaspiro[3.3]heptan-2-yl)acetamide (70 mg, 0.33 mmol) (Intermediate 4) in MeCN (3 mL) was added 2-chloro-N-(3,5-dichlorophenyl)acetamide (79 mg, 0.33 mmol) (Intermediate 6) and K₂CO₃ (137 mg, 0.99 mmol). The reaction was stirred at room temperature overnight and then concentrated in vacuo. The residue was taken up in EtOAc (3×5 mL), and the organics were washed with water (2×3 mL) and then saturated brine solution (3 mL). The combined organics were separated and dried (Na₂SO₄) before concentration in vacuo. The resultant residue was purified by flash column chromatography eluting with 5% MeOH in DCM to provide 2-[2-[2-(tert-butylamino)-2-oxo-ethyl]-2,6-diazaspiro[3.3]heptan-6-yl]-N-(3,5-dichlorophenyl) acetamide (26 mg, 18% yield) as a yellow solid.

MS obsd. (ESI+): [(M+H)⁺]: 413.3 Rₜ: 1.51 min (Method 2). ¹H NMR (400 MHz, DMSO-d6) δ: 9.99 (s, 1H), 7.75 (d, J=1.6 Hz, 2H), 7.27 (s, 1H), 7.03 (s, 1H), 3.36 (s, 4H), 3.32-3.30 (s, 4H), 3.20 (s, 2H), 2.89 (s, 2H), 1.24 (s, 9H).

The following compounds as listed in Table 1 below were prepared similarly, and certain compounds (as indicated) were tested for Human Cav3.1 calcium channel activity via a patch clamp assay as disclosed herein.

Inhibition of the T-type voltage gated calcium channel (Cav3.1) was evaluated using a HEK-293 natClytin/TASK1+Cav3.1 cell line. Currents were recorded using the SyncroPatch 384PE automated, patch clamp system. Pulse generation and data collection were performed with PatchController384 V1.3.0 and DataController384 V1.2.1 (Nanion Technologies). Off-line analysis was performed using Excel and Graphpad Prism (V 8.4.2) with complete data files uploaded to Dotmatics. The access resistance and apparent membrane capacitance were estimated using built-in protocols. Current was recorded in whole cell configuration from a population of cells. The cells were lifted, triturated, and resuspended at 800,000 cells/ml. The cells were allowed to recover in the cell hotel prior to experimentation. Currents were recorded at room temperature. The external solution contained the following (in mM): NaCl 80, NMDG 60, KCl 4, MgCl₂ 1, CaCl₂ 6, glucose 5 and HEPES 10 (pH=7.4, Osmolarity ˜300 mOsm). The extracellular solution was used as the wash, reference and compound delivery solution. The internal solution contained the following (in mM): CsF 110, CsCl 10, NaCl 10, EGTA 10, HEPES 10 (pH=7.2, Osmolarity ˜295 mOsm). The compound plate was created at 2× concentrated in the extracellular solution. The compound was diluted to 1:2 when added to the recording well. The amount of DM50 in the extracellular solution was held constant at the level used for the highest tested concentration. For the voltage clamp experiments on Cav3.1, data were sampled at 10 KHz. After establishment of the seal and the passage in the whole cell configuration, the cells were held at −120 mV. Cav3.1 current was evoked using a 100 ms step to −20 mV (to measure resting state block), followed by a 1600 ms step to −65 mV and a second 100 ms step to −20 mV (to measure voltage dependent block). The voltage protocol was applied every 15 seconds in the absence and in the presence of the compounds under investigation. 2.5 mM Nickel was used to completely inhibit Cav3.1 current to allow for offline subtraction of non-Cav3.1 current. Current amplitude (pA) was measured in the peak 1 and 2. The average of last 3 sweeps of each liquid period (vehicle, compound under investigation, full block) was calculated. Nickel-sensitive current was used to calculate the % of inhibition in the presence of the compound under investigation. In the Tables 1 and 2 below, the human Cav3.1 IC₅₀ is indicated by either A (<1 μM), B (1 μM-<10 μM), or C (≥10 μM).

TABLE 1
Examples 1-8

| | Human | MS | | |
|---|---|---|---|---|
| | Cav3.1 | obsv. | LCMS Rt | HPLC Rt |
| | IC₅₀ | (ESI+): | (min)/QC | (min)/QC |
| Structure | (μM) | [(M + H)⁺] | (Method) | (Method) |
| 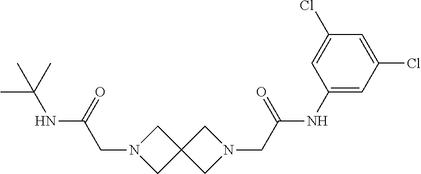 [SMILES: CC(C)(C)NC(=O)CN1CC2(CN(CC(=O)Nc3cc(Cl)cc(Cl)c3)C2)C1] | A | 413.3 | 1.51 (2) | NA |
| Example 1 | | | | |
| 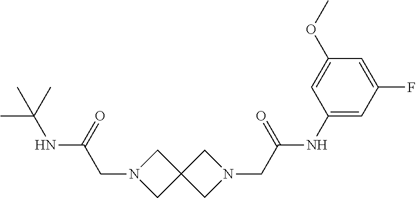 [SMILES: COc1cc(F)cc(NC(=O)CN2CC3(C2)CN(CC(=O)NC(C)(C)C)C3)c1] | B | 393.3 | 1.48 (2) | 1.77 (B) |
| Example 2 | | | | |
| 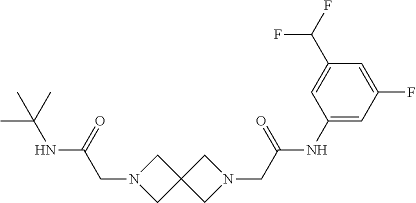 [SMILES: CC(C)(C)NC(=O)CN1CC2(CN(CC(=O)Nc3cc(F)cc(C(F)F)c3)C2)C1] | B | 413.3 | 1.55 (2) | 2.27 (B) |
| Example 3 | | | | |
| 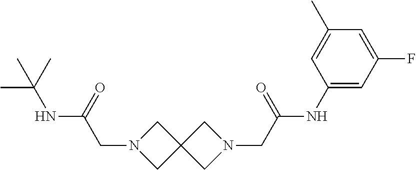 [SMILES: Cc1cc(F)cc(NC(=O)CN2CC3(C2)CN(CC(=O)NC(C)(C)C)C3)c1] | B | 377.3 | 1.54 (2) | 1.82 (B) |
| Example 4 | | | | |
| 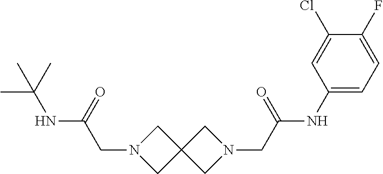 [SMILES: CC(C)(C)NC(=O)CN1CC2(CN(CC(=O)Nc3ccc(F)c(Cl)c3)C2)C1] | B | 397.3 | 1.55 (2) | 2.25 (B) |
| Example 5 | | | | |
| 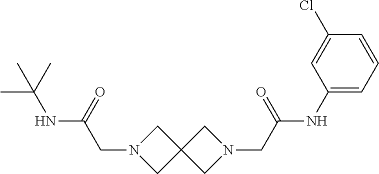 [SMILES: CC(C)(C)NC(=O)CN1CC2(CN(CC(=O)Nc3cccc(Cl)c3)C2)C1] | B | 379.3 | 1.53 (2) | 1.81 (B) |
| Example 6 | | | | |
| 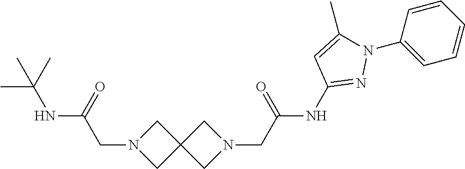 [SMILES: Cc1cc(NC(=O)CN2CC3(C2)CN(CC(=O)NC(C)(C)C)C3)nn1-c1ccccc1] | C | 425.4 | 1.49 (2) | 2.25 (B) |
| Example 7 | | | | |
| 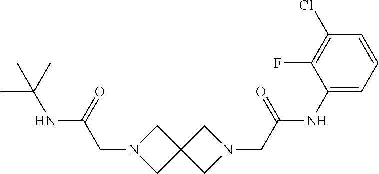 [SMILES: CC(C)(C)NC(=O)CN1CC2(CN(CC(=O)Nc3cccc(Cl)c3F)C2)C1] | C | 397.3 | 1.59 (2) | 2.13 (B) |
| Example 8 | | | | |

¹H NMR (400 MHz, DMSO-d6) δ data for Examples 2-8 were as follows:

Example 2: 9.79 (s, 1H), 7.17 (dd, J=11.2, 1.8 Hz, 1H), 7.08 (s, 1H), 7.03 (d, J=12.8 Hz, 1H), 6.51 (m, 1H), 3.73 (s, 3H), 3.35 (s, 4H), 3.30 (s, 4H), 3.16 (s, 2H), 2.87 (s, 2H), 1.24 (s, 9H).

Example 3: 10.06 (s, 1H), 7.72 (s, 1H), 7.70 (s, 1H), 7.10 (d, J=8.4 Hz, 1H), 7.02 (obs. s, 1H), 7.02 (t, J=56 Hz, 1H), 3.37 (s, 4H), 3.31 (s, 4H), 3.20 (s, 2H), 2.88 (s, 2H), 1.24 (s, 9H).

Example 4: 9.74 (s, 1H), 7.39 (d, J=11.6 Hz, 1H), 7.21 (s, 1H), 7.02 (s, 1H), 6.71 (d, J=9.6 Hz, 1H), 3.36 (s, 4H), 3.31 (s, 4H), 3.16 (s, 2H), 2.89 (s, 2H), 2.27 (s, 3H), 1.24 (s, 9H).

Example 5: 9.86 (s, 1H), 7.94 (dd, J=6.8, 2.4 Hz, 1H), 7.61-7.53 (m, 1H), 7.35 (s, 1H), 7.02 (s, 1H), 3.36 (s, 4H), 3.31 (s, 4H), 3.17 (s, 2H), 2.88 (s, 2H), 1.24 (s, 9H).

Example 6: 9.83 (s, 1H), 7.83 (s, 1H), 7.52 (d, J=8.2 Hz, 1H), 7.31 (t, J=8.2 Hz, 1H), 7.10 (d, J=7.8 Hz, 1H), 7.00 (s, 1H), 3.36 (s, 4H), 3.30 (s, 4H), 3.17 (s, 2H), 2.87 (s, 2H), 1.24 (s, 9H).

Example 7: 10.05 (s, 1H), 7.50 (d, J=3.8 Hz, 4H), 7.42-7.35 (m, 1H), 7.00 (s, 1H), 6.56 (s, 1H), 3.34 (s, 4H), 3.29 (s, 4H), 3.15 (s, 2H), 2.86 (s, 2H), 2.32 (s, 3H), 1.24 (s, 9H).

Example 8: 9.60 (s, 1H), 7.80 (t, J=7.6 Hz, 1H), 7.34 (t, J=7.6 Hz, 1H), 7.19 (t, J=8.2 Hz, 1H), 7.01 (s, 1H), 3.39 (s, 4H), 3.30 (s, 4H), 3.23 (s, 2H), 2.87 (s, 2H), 1.24 (s, 9H).

### Example 9—Preparation of N-(tert-butyl)-2-(6-((7-chloro-5-fluoro-1H-benzo[d]imidazol-2-yl)methyl)-2,6-diazaspiro[3.3]heptan-2-yl)acetamide

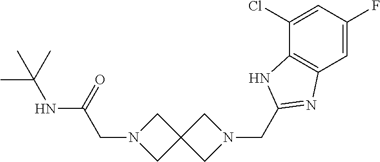 [SMILES: CC(C)(C)NC(=O)CN1CC2(C1)CN(Cc1nc3cc(F)cc(Cl)c3[nH]1)C2]

### Step 1: Preparation of 4-chloro-2-(chloromethyl)-6-fluoro-1H-benzo[d]imidazole (Intermediate 7)

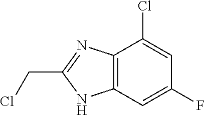 [SMILES: Fc1cc(Cl)c2nc(CCl)[nH]c2c1]

To a solution of 3-chloro-5-fluoro-benzene-1,2-diamine (2.0 g, 12.45 mmol) in DCM (21 mL) was added 2-chloro-1,1,1-trimethoxy-ethane (2.50 g, 16.19 mmol) and p-toluenesulfonic acid (2.38 g, 1.25 mmol). The reaction solution was stirred at room temperature for 12 hours. The reaction was concentrated to dryness, and the residue was taken up in DCM (80 mL). The organics were washed with water (2×13 mL) and then saturated brine solution (13 mL). The organics were then separated and dried (Na₂SO₄) before concentration in vacuo. The crude was purified by silica gel chromatography eluting with 1-2% MeOH in DCM to give 4-chloro-2-(chloromethyl)-6-fluoro-1H-benzimidazole (2.5 g, 84% yield, 92% purity) as an off-white solid. MS obsd. (ESI+): [(M+H)⁺]: 218.9. Rₜ: 0.83 min (Method 1).

### Step 2: Preparation of N-(tert-butyl)-2-(6-((7-chloro-5-fluoro-1H-benzo[d]imidazol-2-yl) methyl)-2,6-diazaspiro[3.3]heptan-2-yl)acetamide (Example 9)

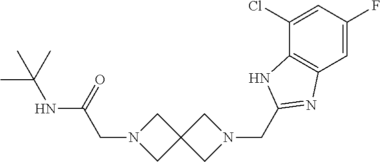 [SMILES: CC(C)(C)NC(=O)CN1CC2(C1)CN(Cc1nc3cc(F)cc(Cl)c3[nH]1)C2]

### N-(tert-butyl)-2-(6-((7-chloro-5-fluoro-1H-benzo[d]imidazol-2-yl)methyl)-2,6-diazaspiro[3.3]heptan-2-yl)acetamide

To a solution of N-(tert-butyl)-2-(2,6-diazaspiro[3.3]heptan-2-yl)acetamide (150 mg, 0.71 mmol) (Intermediate 4) in MeCN (10 mL) was added Et₃N (0.30 mL, 2.13 mmol) and 4-chloro-2-(chloromethyl)-6-fluoro-1H-benzo[d]imidazole (156 mg, 0.71 mmol) (Intermediate 7). The reaction was concentrated to dryness, and the residue was partitioned between water (3 mL) and EtOAc (3×10 mL). The organics were washed with water (3 mL) and then saturated brine solution (3 mL). The organics were then separated and dried (Na₂SO₄) before concentration in vacuo. The crude was purified by silica gel chromatography eluting with 10% MeOH in DCM to give N-(tert-butyl)-2-(6-((7-chloro-5-fluoro-1H-benzo[d]imidazol-2-yl) methyl)-2,6-diazaspiro[3.3]heptan-2-yl)acetamide (38 mg, 13% yield, 96.9% purity) as an off-white solid.

MS obsd. (ESI+): [(M+H)⁺]: 394.3. R: 1.41 min (Method 2). ¹H NMR (400 MHz, DMSO-d6) δ 12.69 (s, 1H), 7.20 (d, J=9.6 Hz, 2H), 7.10 (s, 1H), 3.75 (s, 2H), 3.37 (s, 4H), 3.32 (s, 4H), 2.96 (s, 2H), 1.24 (s, 9H).

### Example 10: Preparation of N-(3-chloro-5-fluoro-phenyl)-2-[2-[2-(1,1-dimethylpropyl amino)-2-oxo-ethyl]-2,6-diazaspiro[3.3]heptan-6-yl]acetamide

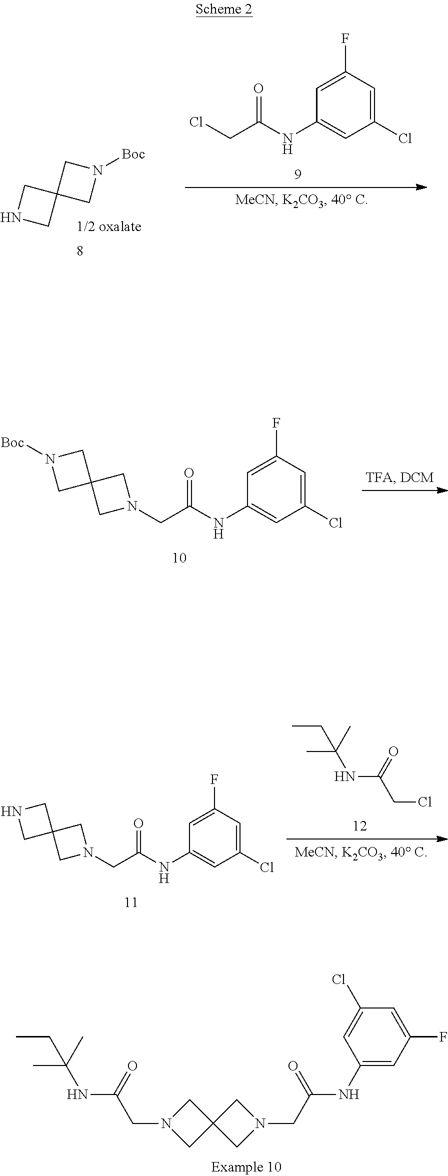

### General Method 2:

### Step 1: Preparation of 6-[2-(3-chloro-5-fluoro-anilino)-2-oxo-ethyl]-2,6-diazaspiro[3.3]heptane-2-carboxylate (Intermediate 10)

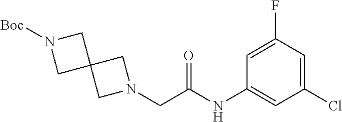 [SMILES: CC(C)(C)OC(=O)N1CC2(CN(CC(=O)Nc3cc(F)cc(Cl)c3)C2)C1]

To the solution of tert-butyl 2,6-diazaspiro[3.3]heptane-2-carboxylate oxalic acid (3.0 g, 10.4 mmol) (Intermediate 8) in MeCN (30 mL) was added 2-chloro-N-(3-chloro-5-fluoro-phenyl)acetamide (2.54 g, 11.5 mmol) (Intermediate 9) and K₂CO₃ (4.31 g, 31.2 mmol). The reaction mixture was stirred at 40° C. for 6 hours. The reaction was concentrated in vacuo, and the residue taken up in EtOAc (100 mL). The organic layer was washed with water (2×150 ml) and saturated brine solution (100 mL) and then separated, dried (Na₂SO₄), and concentrated in vacuo. The resultant residue was purified by flash column chromatography eluting with 10% EtOAc in PE to afford tert-butyl 6-[2-(3-chloro-5-fluoro-anilino)-2-oxo-ethyl]-2,6-diazaspiro[3.3]heptane-2-carboxylate (3.7 g, 83% yield, 90% purity) as a yellow solid. MS obsd. (ESI+): [(M+H)⁺]: 385.7. Rₜ:1.77 min (Method 2).

### Step 2: Preparation of N-(3-chloro-5-fluoro-phenyl)-2-(2,6-diazaspiro[3.3]heptan-2-yl) acetamide (Intermediate 11)

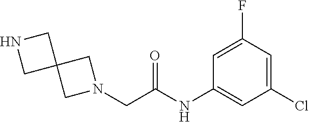 [SMILES: O=C(CN1CC2(CNC2)C1)Nc1cc(F)cc(Cl)c1]

To the solution of tert-butyl 6-[2-(3-chloro-5-fluoro-anilino)-2-oxo-ethyl]-2,6-diazaspiro[3.3]heptane-2-carboxylate (3.7 g, 9.64 mmol) (Intermediate 10) in DCM (30 mL) was added TFA (10 mL, 71 mmol). The reaction mixture was stirred at room temperature for 2 hours and then concentrated to dryness. The pH of the residue was adjusted to 8-9 prior to extracting with 10% DCM/MeOH (5×45 mL). The combined organic phase was dried with Na₂SO₄, filtered, and concentrated in vacuo to provide N-(3-chloro-5-fluoro-phenyl)-2-(2,6-diazaspiro[3.3]heptan-2-yl)acetamide (2.2 g, 6.97 mmol, 72.4% yield, 90% purity) as a yellow oil. MS obsd. (ESI+): [(M+H)⁺]: 284.0. Rₜ: 0.70 min (Method 1).

### Step 3: Preparation of N-(3-chloro-5-fluoro-phenyl)-2-[2-[2-(1,1-dimethylpropylamino)-2-oxo-ethyl]-2,6-diazaspiro[3.3]heptan-6-yl]acetamide (Example 10)

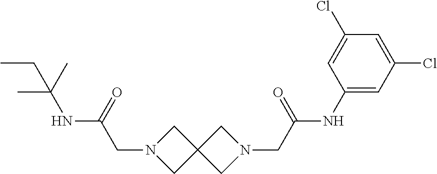 [SMILES: CCC(C)(C)NC(=O)CN1CC2(CN(CC(=O)Nc3cc(Cl)cc(Cl)c3)C2)C1]

To the solution of N-(3-chloro-5-fluoro-phenyl)-2-(2,6-diazaspiro[3.3]heptan-2-yl)acetamide (60 mg, 0.21 mmol) (Intermediate 11) in MeCN (1 mL) was added 2-chloro-N-(1,1-dimethylpropyl)acetamide (38 mg, 0.23 mmol) (Intermediate 12) and K₂CO₃ (88 mg, 0.63 mmol). The reaction mixture was stirred at room temperature for 16 hours and then concentrated in vacuo. The residue was taken up in DCM (10 mL), and the organic layer was washed with water (2×8 mL) and then saturated brine solution (10 mL). The organic layer was separated and dried (Na₂SO₄) before concentration in vacuo. The resultant residue was purified by prep-HPLC (15% to 95% [MeCN/0.05% NH₃ in H₂O] in MeCN over 15 mins) to give, after lyophilization, the title compound N-(3-chloro-5-fluoro-phenyl)-2-[2-[2-(1,1-dimethylpropylamino)-2-oxo-ethyl]-2,6-diazaspiro[3.3]heptan-6-yl]acetamide (33 mg, 37% yield, 97.3% purity) as a white solid.

MS obsd. (ESI+): [(M+H)⁺]: 411.4. Rₜ:1.68 min (Method 2). ¹H NMR (400 MHz, DMSO-d6) δ 10.01 (s, 1H), 7.62 (s, 1H), 7.58-7.50 (m, 1H), 7.11-7.08 (m, 1H), 6.86 (s, 1H), 3.36 (s, 4H), 3.30 (s, 4H), 3.19 (s, 2H), 2.88 (s, 2H), 1.62 (q, J=7.4 Hz, 2H), 1.19 (s, 6H), 0.75 (t, J=7.4 Hz, 3H).

The following compounds as listed in Table 2 were prepared similarly, and certain compounds (as indicated) were tested for Human Cav3.1 calcium channel activity via the methods disclosed in Example 1.

TABLE 2
Examples 9-43

| | Human | MS | | |
|---|---|---|---|---|
| | Cav3.1 | obsv. | LCMS Rt | HPLC Rt |
| | IC₅₀ | (ESI+): | (min)/QC | (min)/QC |
| Structure | (μM) | [(M + H)⁺] | (Method) | (Method) |
| 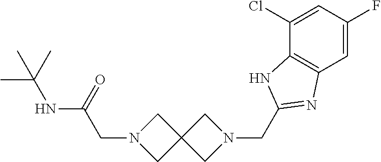 [SMILES: CC(C)(C)NC(=O)CN1CC2(C1)CN(Cc1nc3cc(F)cc(Cl)c3[nH]1)C2] | A | 394.3 | 1.41 (2) | NA |
| Example 9 | | | | |
| 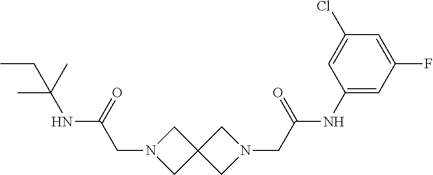 [SMILES: CCC(C)(C)NC(=O)CN1CC2(CN(CC(=O)Nc3cc(F)cc(Cl)c3)C2)C1] | A | 411.4 | 1.68 (2) | NA |
| Example 10 | | | | |
| 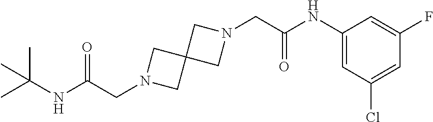 [SMILES: CC(C)(C)NC(=O)CN1CC2(CN(CC(=O)Nc3cc(F)cc(Cl)c3)C2)C1] | A | 397.4 | 1.55 (2) | 1.36 (A) |
| Example 11 | | | | |
| 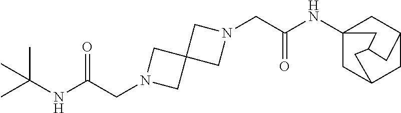 [SMILES: CC(C)(C)NC(=O)CN1CC2(C1)CN(CC(=O)NC13CC4CC(CC(C4)C1)C3)C2] | B | 403.3 | 1.61 (2) | 4.23 (B) |
| Example 12 | | | | |
| 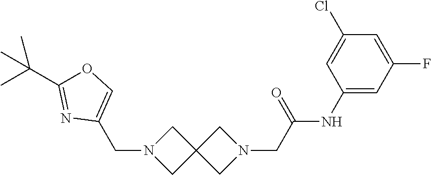 [SMILES: CC(C)(C)c1nc(CN2CC3(CN(CC(=O)Nc4cc(F)cc(Cl)c4)C3)C2)co1] | A | 421.2 | 1.73 (2) | 3.23 (B) |
| Example 13 | | | | |
| 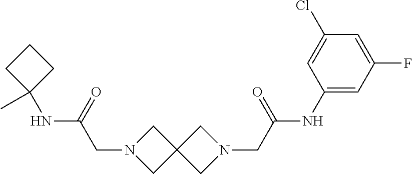 [SMILES: CC1(NC(=O)CN2CC3(CN(CC(=O)Nc4cc(F)cc(Cl)c4)C3)C2)CCC1] | A | 409.2 | 1.61 (2) | 2.89 (B) |
| Example 14 | | | | |
| 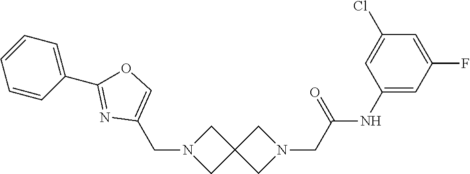 [SMILES: O=C(CN1CC2(C1)CN(Cc1coc(-c3ccccc3)n1)C2)Nc1cc(F)cc(Cl)c1] | A | 441.2 | 1.75 (2) | 3.30 (B) |
| Example 15 | | | | |
| 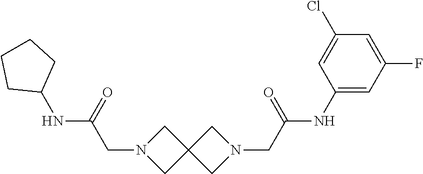 [SMILES: O=C(CN1CC2(C1)CN(CC(=O)NC1CCCC1)C2)Nc1cc(F)cc(Cl)c1] | B | 409.2 | 1.59 (2) | 2.84 (B) |
| Example 16 | | | | |
| 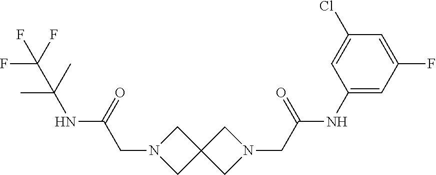 [SMILES: CC(C)(NC(=O)CN1CC2(CN(CC(=O)Nc3cc(F)cc(Cl)c3)C2)C1)C(F)(F)F] | B | 451.2 | 1.71 (2) | 3.17 (B) |
| Example 17 | | | | |
| 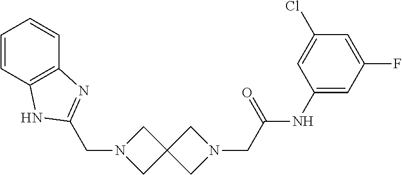 [SMILES: O=C(CN1CC2(C1)CN(Cc1nc3ccccc3[nH]1)C2)Nc1cc(F)cc(Cl)c1] | A | 414.3 | 1.50 (2) | 3.16 (B) |
| Example 18 | | | | |
| 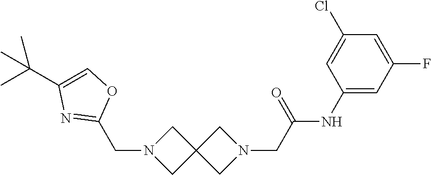 [SMILES: CC(C)(C)c1coc(CN2CC3(CN(CC(=O)Nc4cc(F)cc(Cl)c4)C3)C2)n1] | B | 421.7 | 1.76 (2) | 3.23 (B) |
| Example 19 | | | | |
| 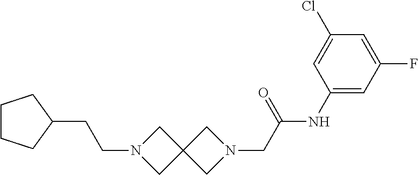 [SMILES: O=C(CN1CC2(CN(CCC3CCCC3)C2)C1)Nc1cc(F)cc(Cl)c1] | A | 380.2 | 2.03 (2) | 3.82 (B) |
| Example 20 | | | | |
| 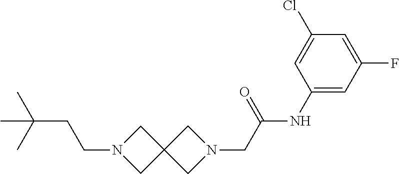 [SMILES: CC(C)(C)CCN1CC2(C1)CN(CC(=O)Nc1cc(F)cc(Cl)c1)C2] | A | 368.3 | 1.93 (2) | 3.65 (B) |
| Example 21 | | | | |
| 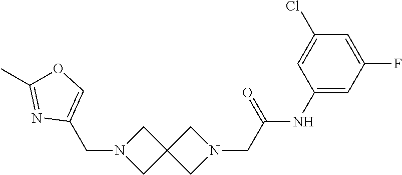 [SMILES: Cc1nc(CN2CC3(CN(CC(=O)Nc4cc(F)cc(Cl)c4)C3)C2)co1] | B | 379.2 | 1.47 (2) | 2.51 (B) |
| Example 22 | | | | |
| 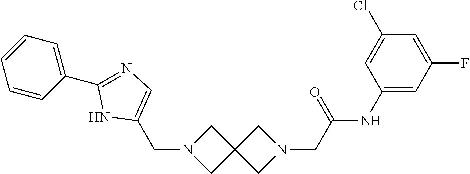 [SMILES: O=C(CN1CC2(C1)CN(Cc1cnc(-c3ccccc3)[nH]1)C2)Nc1cc(F)cc(Cl)c1] | A | 440.2 | 1.55 (2) | 2.71 (B) |
| Example 23 | | | | |
| 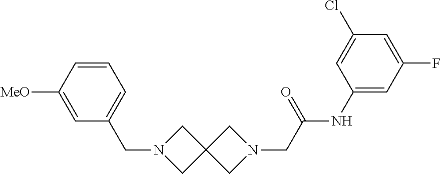 [SMILES: COc1cccc(CN2CC3(CN(CC(=O)Nc4cc(F)cc(Cl)c4)C3)C2)c1] | B | 404.7 | 1.75 (2) | 3.29 (B) |
| Example 24 | | | | |
| 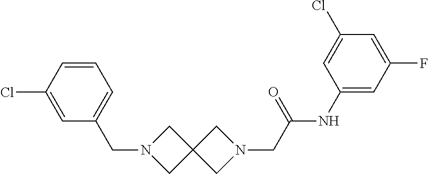 [SMILES: O=C(CN1CC2(C1)CN(Cc1cccc(Cl)c1)C2)Nc1cc(F)cc(Cl)c1] | A | 408.2 | 1.87 (2) | 3.62 (B) |
| Example 25 | | | | |
| 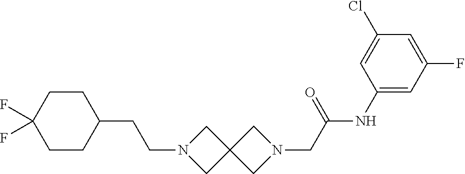 [SMILES: O=C(CN1CC2(CN(CCC3CCC(F)(F)CC3)C2)C1)Nc1cc(F)cc(Cl)c1] | A | 430.3 | 1.86 (2) | 3.60 (B) |
| Example 26 | | | | |
| 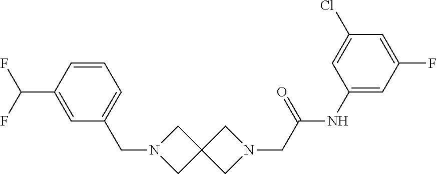 [SMILES: O=C(CN1CC2(C1)CN(Cc1cccc(C(F)F)c1)C2)Nc1cc(F)cc(Cl)c1] | A | 424.2 | 1.79 (2) | 3.44 (B) |
| Example 27 | | | | |
| 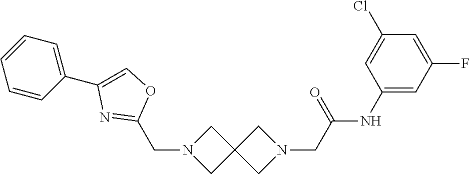 [SMILES: O=C(CN1CC2(C1)CN(Cc1nc(-c3ccccc3)co1)C2)Nc1cc(F)cc(Cl)c1] | B | 442.9 | 1.75 (2) | 3.30 (B) |
| Example 28 | | | | |
| 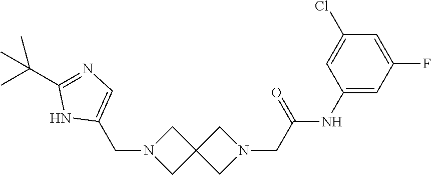 [SMILES: CC(C)(C)c1ncc(CN2CC3(CN(CC(=O)Nc4cc(F)cc(Cl)c4)C3)C2)[nH]1] | B | 420.3 | 1.54 (2) | 2.61 (B) |
| Example 29 | | | | |
| 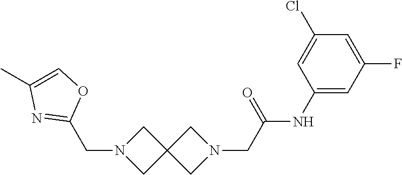 [SMILES: Cc1coc(CN2CC3(CN(CC(=O)Nc4cc(F)cc(Cl)c4)C3)C2)n1] | B | 379 | 1.48 (2) | 2.58 (B) |
| Example 30 | | | | |
| 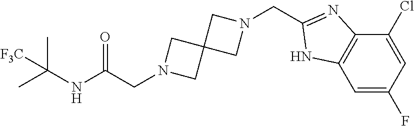 [SMILES: CC(C)(NC(=O)CN1CC2(C1)CN(Cc1nc3c(Cl)cc(F)cc3[nH]1)C2)C(F)(F)F] | A | 448.3 | 1.48 (2) | 2.63 (B) |
| Example 31 | | | | |
| 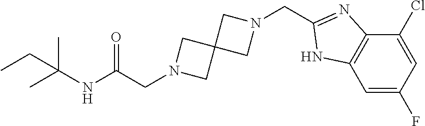 [SMILES: CCC(C)(C)NC(=O)CN1CC2(C1)CN(Cc1nc3c(Cl)cc(F)cc3[nH]1)C2] | A | 408.3 | 1.45 (2) | 2.57 (A) |
| Example 32 | | | | |
| 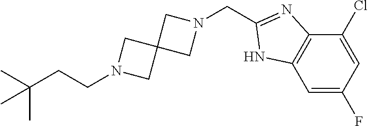 [SMILES: CC(C)(C)CCN1CC2(C1)CN(Cc1nc3c(Cl)cc(F)cc3[nH]1)C2] | A | 365.4 | 1.65 (2) | 3.00 (B) |
| Example 33 | | | | |
| 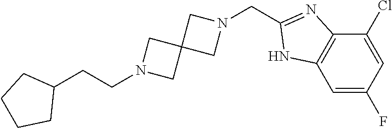 [SMILES: Fc1cc(Cl)c2nc(CN3CC4(CN(CCC5CCCC5)C4)C3)[nH]c2c1] | A | 377.4 | 1.66 (2) | 3.21 (B) |
| Example 34 | | | | |
| 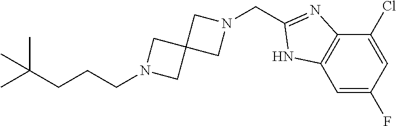 [SMILES: CC(C)(C)CCCN1CC2(C1)CN(Cc1nc3c(Cl)cc(F)cc3[nH]1)C2] | A | 379.3 | 1.72 (2) | 3.34 (B) |
| Example 35 | | | | |
| 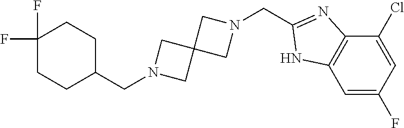 [SMILES: Fc1cc(Cl)c2nc(CN3CC4(C3)CN(CC3CCC(F)(F)CC3)C4)[nH]c2c1] | A | 413.3 | 1.55 (2) | 2.87 (B) |
| Example 36 | | | | |
| 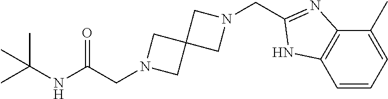 [SMILES: Cc1cccc2[nH]c(CN3CC4(CN(CC(=O)NC(C)(C)C)C4)C3)nc12] | B | 355.48 | 1.30 (2) | 2.15 (B) |
| Example 37 | | | | |
| 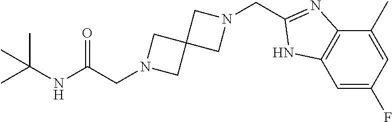 [SMILES: Cc1cc(F)cc2[nH]c(CN3CC4(CN(CC(=O)NC(C)(C)C)C4)C3)nc12] | B | 373.4 | 1.34 (2) | 2.27 (B) |
| Example 38 | | | | |
| 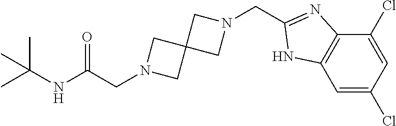 [SMILES: CC(C)(C)NC(=O)CN1CC2(C1)CN(Cc1nc3c(Cl)cc(Cl)cc3[nH]1)C2] | A | 410.3 | 1.46 (2) | 2.52 (B) |
| Example 39 | | | | |
| 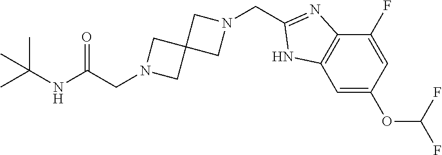 [SMILES: CC(C)(C)NC(=O)CN1CC2(C1)CN(Cc1nc3c(F)cc(OC(F)F)cc3[nH]1)C2] | B | 426.4 | 1.39 (2) | 2.41 (B) |
| Example 40 | | | | |
| 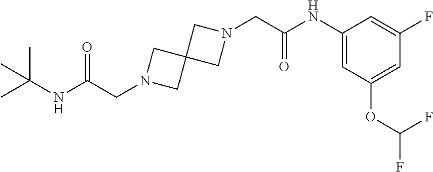 [SMILES: CC(C)(C)NC(=O)CN1CC2(CN(CC(=O)Nc3cc(F)cc(OC(F)F)c3)C2)C1] | B | 429.3 | 1.56 (2) | 1.54 (A) |
| Example 41 | | | | |
| 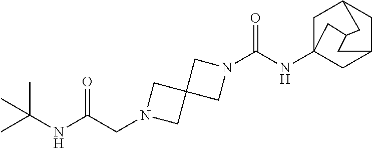 [SMILES: CC(C)(C)NC(=O)CN1CC2(C1)CN(C(=O)NC13CC4CC(CC(C4)C1)C3)C2] | | 389.2 | 1.58 (2) | 4.22 (B) |
| Example 42 | | | | |
| 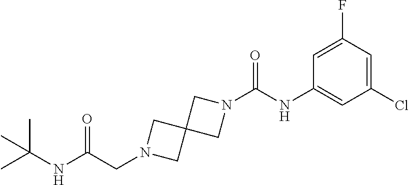 [SMILES: CC(C)(C)NC(=O)CN1CC2(C1)CN(C(=O)Nc1cc(F)cc(Cl)c1)C2] | A | 383.3 | 1.52 (2) | 2.34 (A) |
| Example 43 | | | | |

¹H NMR (400 MHz, DMSO-d6) δ data for Examples 11-29 were as follows:

Example 11: 10.02 (s, 1H), 7.64-7.59 (m, 1H), 7.58-7.51 (m, 1H), 7.14-7.00 (m, 2H), 3.37 (s, 4H), 3.35 (s, 4H), 3.20 (s, 2H), 2.93 (s, 2H), 1.24 (s, 9H).

Example 12: 6.99 (s, 1H), 6.85 (s, 1H), 3.26 (s, 8H), 2.85 (d, J=3.1 Hz, 4H), 2.00 (s, 3H), 1.89 (d, J=2.6 Hz, 6H), 1.61 (s, 6H), 1.24 (s, 9H).

Example 13: 10.00 (s, 1H), 7.71 (s, 1H), 7.62 (s, 1H), 7.56-7.53 (m, 1H), 7.11-7.08 (m, 1H), 3.36-3.35 (m, 6H), 3.25 (s, 4H), 3.19 (s, 2H), 1.28 (s, 9H).

Example 14: 10.03 (s, 1H), 7.62 (s, 1H), 7.55 (dt, J=11.2, 2.0 Hz, 1H), 7.49 (s, 1H), 7.09 (dt, J=8.6, 2.0 Hz, 1H), 3.36 (s, 4H), 3.30 (s, 4H), 3.19 (s, 2H), 2.88 (s, 2H), 2.24-2.17 (m, 2H), 1.90-1.82 (m, 2H), 1.78-1.69 (m, 2H), 1.33 (s, 3H).

Example 15: 10.03 (s, 1H), 7.99 (s, 1H), 7.96-7.94 m, 2H), 7.62 (s, 1H), 7.56-7.52 (m, 4H), 7.11-7.08 (m, 1H), 3.46 (s, 2H), 3.37 (s, 4H), 3.30 (s, 4H), 3.20 (s, 2H).

Example 16: 10.03 (s, 1H), 7.62 (s, 1H), 7.56-7.53 (m, 1H), 7.45 (d, J=7.6 Hz, 1H), 7.11-7.08 (m, 1H), 4.04-3.89 (m, 1H), 3.36 (s, 5H), 3.29 (s, 5H), 3.19 (s, 2H), 2.91 (s, 2H), 1.78-1.72 (m, 2H), 1.67-1.57 (m, 2H), 1.53-1.46 (m, 2H), 1.42-1.31 (m, 2H).

Example 17: 10.39 (s, 1H), 7.55-7.45 (m, 4H), 7.42-7.32 (m, 1H), 7.01 (s, 1H), 6.56 (s, 1H), 3.03 (s, 2H), 2.94-2.93 (m, 4H), 2.31 (s, 4H), 1.93-1.90 (m, 2H), 1.68-1.65 (m, 2H), 1.47-1.31 (m, 4H), 1.26 (s, 9H).

Example 18: 12.23 (s, 1H), 10.01 (s, 1H), 7.62 (s, 1H), 7.56-7.51 (m, J=9.0 Hz, 2H), 7.41-7.39 (m, 1H), 7.15-7.07 (m, 3H), 3.74 (s, 2H), 3.38 (s, 4H), 3.35 (s, 4H), 3.20 (s, 2H).

Example 19: 10.00 (s, 1H), 7.67 (s, 1H), 7.62 (s, 1H), 7.55 (dt, J=11.2, 2.0 Hz, 1H), 7.09 (dt, J=8.6, 2.0 Hz, 1H), 3.55 (s, 2H), 3.34 (s, 4H), 3.32 (s, 4H), 3.19 (s, 2H), 1.18 (s, 9H).

Example 20: 10.01 (s, 1H), 7.62 (s, 1H), 7.54 (d, J=11.2 Hz, 1H), 7.09 (d, J=8.6 Hz, 1H), 3.33 (s, 4H), 3.19 (s, 2H), 3.16 (s, 4H), 2.35-2.26 (m, 2H), 1.73-1.65 (m, 3H), 1.58-1.52 (m, 2H), 1.49-1.44 (m, 2H), 1.26-1.21 (m, 2H), 1.06-0.98 (m, 2H).

Example 21: 10.06 (s, 1H), 8.20 (s, 2H), 7.61 (s, 1H), 7.54 (dt, J=11.2, 2.0 Hz, 1H), 7.10 (dt, J=8.6, 2.0 Hz, 1H), 3.62 (s, 4H), 3.41 (s, 4H), 3.22 (s, 2H), 2.68-2.67 (m, 2H), 1.32-1.16 (m, 2H), 0.86 (s, 9H).

Example 22: 10.00 (s, 1H), 7.70 (s, 1H), 7.62 (s, 1H), 7.56-7.53 (m, 1H), 7.11-7.08 (m, 1H), 3.34 (s, 4H), 3.31 (s, 2H), 3.23 (s, 4H), 3.19 (s, 2H), 2.34 (s, 3H).

Example 23: 12.34 (d, J=37.8 Hz, 1H), 10.00 (s, 1H), 7.93-7.87 (m, 2H), 7.62 (s, 1H), 7.55 (d, J=11.2 Hz, 1H), 7.43-7.39 (m, 2H), 7.33-7.29 (m 1H), 7.11-7.07 (m, 1H), 6.91 (d, J=72.5 Hz, 1H), 3.45 (d, J=30.6 Hz, 2H), 3.35 (s, 4H), 3.25 (s, 2H), 3.22 (s, 2H), 3.19 (s, 2H).

Example 24: 10.00 (s, 1H), 7.62 (s, 1H), 7.56-7.53 (m, 1H), 7.22-7.18 (m, 1H), 7.12-7.08 (m, 1H), 6.82-6.78 (m, 3H), 3.73 (s, 3H), 3.46 (s, 2H), 3.36 (s, 4H), 3.21 (s, 4H), 3.19 (s, 2H).

Example 25: 10.00 (s, 1H), 7.62 (s, 1H), 7.54 (d, J=11.2 Hz, 1H), 7.36-7.30 (m, 1H), 7.29 (s, 2H), 7.21 (d, J=7.2 Hz, 1H), 7.09 (d, J=8.6 Hz, 1H), 3.50 (s, 2H), 3.36 (s, 4H), 3.23 (s, 4H), 3.19 (s, 2H).

Example 26: 10.01 (s, 1H), 7.62 (s, 1H), 7.54 (d, J=11.2 Hz, 1H), 7.09 (d, J=8.6 Hz, 1H), 3.33 (s, 4H), 3.19 (s, 2H), 3.13 (s, 4H), 2.30 (t, J=7.2 Hz, 2H), 1.95-1.93 (m, 2H), 1.82-1.78 (m, 1H), 1.72-1.69 (m, 4H), 1.19-1.14 (m, 2H), 1.11-1.09 (m, 2H).

Example 27: 9.99 (s, 1H), 7.62 (s, 1H), 7.54 (d, J=11.2 Hz, 1H), 7.46-7.39 (m, 4H), 7.09 (d, J=8.6 Hz, 1H), 7.01 (t, 56.0 Hz, 1H), 3.56 (s, 2H), 3.37 (s, 4H), 3.24 (s, 4H), 3.19 (s, 2H).

Example 28: 10.00 (s, 1H), 8.54 (s, 1H), 7.76 (d, J=7.8 Hz, 2H), 7.62 (s, 1H), 7.55 (d, J=11.2 Hz, 1H), 7.42 (t, J=7.6 Hz, 2H), 7.32 (t, J=7.2 Hz, 1H), 7.09 (d, J=8.6 Hz, 1H), 3.67 (s, 2H), 3.37 (d, J=1.8 Hz, 8H), 3.20 (s, 2H).

Example 29: 11.42-11.32 (m, 1H), 10.00 (s, 1H), 7.62 (s, 1H), 7.54 (d, J=11.2 Hz, 1H), 7.09 (d, J=8.6 Hz, 1H), 6.69-6.47 (m, 1H), 3.37-3.33 (m, 4H), 3.30 (s, 2H), 3.20-3.16 (m, 6H), 1.23 (s, 9H).

Example 30: 10.00 (s, 1H), 7.70 (d, J=1.2 Hz, 1H), 7.62 (s, 1H), 7.55 (dt, J=11.2, 2.0 Hz, 1H), 7.09 (dt, J=8.6, 2.0 Hz, 1H), 3.54 (s, 2H), 3.34 (s, 4H), 3.30 (s, 4H), 3.19 (s, 2H), 2.04 (d, J=1.2 Hz, 3H).

Example 31: 12.69 (s, 1H), 7.51 (s, 1H), 7.21 (s, 2H), 3.75 (s, 2H), 3.34 (s, 8H), 3.00 (s, 2H), 1.47 (s, 6H).

Example 32: 12.70 (s, 1H), 7.20 (d, J=9.3 Hz, 2H), 6.98 (s, 1H), 3.75 (s, 2H), 3.41 (s, 4H), 3.35 (s, 4H), 3.01 (s, 2H), 1.62 (q, J=7.4 Hz, 2H), 1.18 (s, 6H), 0.75 (t, J=7.5 Hz, 3H).

Example 33: 12.72 (s, 1H), 7.20 (d, J=9.7 Hz, 2H), 3.74 (s, 2H), 3.31 (s, 4H), 3.20 (s, 4H), 2.37-2.28 (m, 2H), 1.16-1.07 (m, 2H), 0.84 (s, 9H).

Example 34: 12.73 (s, 1H), 7.21 (d, J=9.7 Hz, 2H), 3.83 (s, 2H), 3.76 (s, 2H), 3.33 (s, 4H), 2.82 (s, 2H), 1.70 (s, 3H), 1.60-1.44 (m, 4H), 1.34 (d, J=4.6 Hz, 2H), 1.23 (s, 2H), 1.05 (s, 2H).

Example 35: 12.73 (s, 1H), 7.21 (d, J=9.8 Hz, 2H), 3.76 (s, 5H), 3.40 (s, 4H), 2.71 (s, 2H), 1.26 (dd, J=15.8, 7.0 Hz, 3H), 1.11 (dd, J=11.1, 5.6 Hz, 2H), 0.85 (s, 9H).

Example 36: 7.20 (ddd, J=12.0, 9.3, 2.1 Hz, 2H), 3.73 (s, 2H), 3.30 (s, 4H), 3.15 (s, 4H), 2.18 (d, J=6.8 Hz, 2H), 1.94 (d, J=7.8 Hz, 2H), 1.79-1.65 (m, 4H), 1.33 (s, 1H), 1.16-0.98 (m, 2H).

Example 37: 12.20 (d, J=46.2 Hz, 1H), 7.27 (dd, J=51.9, 7.8 Hz, 1H), 7.00 (dd, J=9.1, 5.9 Hz, 2H), 6.90 (d, J=6.5 Hz, 1H), 3.71 (d, J=8.9 Hz, 2H), 3.30 (d, J=11.1 Hz, 8H), 2.86 (s, 2H), 2.47 (d, J=6.0 Hz, 3H), 1.23 (s, 9H).

Example 38: 12.34 (d, J=61.7 Hz, 1H), 7.13-6.97 (m, 2H), 6.81 (t, J=11.9 Hz, 1H), 3.71 (s, 2H), 3.33 (s, 8H), 2.93 (s, 2H), 2.47 (d, J=5.7 Hz, 3H), 1.23 (s, 9H).

Example 39: 12.78 (s, 1H), 7.47 (s, 1H), 7.31 (d, J=1.8 Hz, 1H), 7.00 (s, 1H), 3.76 (s, 2H), 3.33 (s, 4H), 3.29 (s, 4H), 2.86 (s, 2H), 1.24 (s, 9H).

Example 40: 7.15-7.10 (m, 1H), 6.88-6.82 (m, 1H), 6.81 (t, J=72 Hz, 1H), 3.81 (s, 2H), 3.48-3.43 (m, 8H), 3.01 (s, 2H), 1.32 (s, 9H).

Example 41: 10.93 (s, 1H), 8.09 (s, 1H), 7.35-7.31 (m, 1H), 7.27 (t, J=73.2 Hz, 1H), 7.23 (s, 1H), 6.94-6.88 (m, 1H), 4.47-4.18 (m, 1OH), 3.91 (s, 2H), 1.27 (s, 9H).

Example 42: 7.02 (s, 1H), 5.42 (s, 1H), 3.78 (s, 4H), 3.29 (s, 4H), 2.86 (s, 2H), 1.98 (s, 3H), 1.87-1.86 (m, 6H), 1.64-1.51 (m, 6H), 1.24 (s, 9H).

Example 43: 8.77 (s, 1H), 7.45-7.44 (m, 1H), 7.42-7.37 (m, 1H), 7.06 (s, 1H), 6.94-6.90 (m, 1H), 4.03 (s, 4H), 3.36 (s, 4H), 2.90 (s, 2H), 1.25 (s, 9H).

What is claimed is:

1. A compound of Formula (IA) having a diazaspiroheptane core: 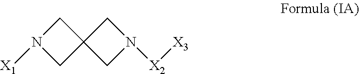
wherein X₁ is a left-hand substitution of the diazaspiroheptane core chosen from: 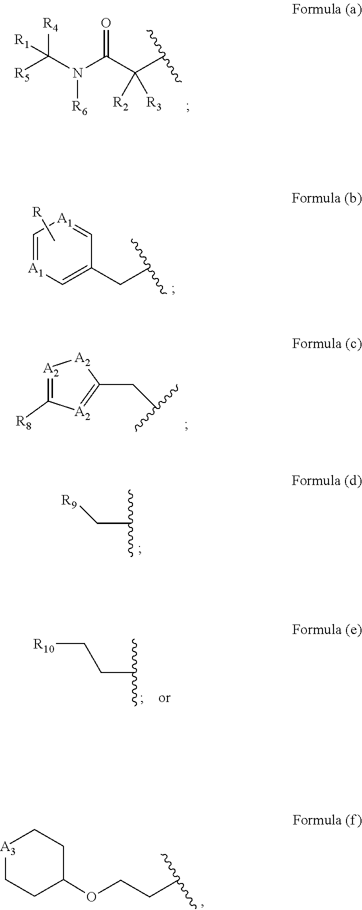
wherein R₁ is chosen from —H, —CH₃, —CH₂OCH₃, —CF₃, —CH₂CH₃, or —(CH₂)₂OCH₃;
R₂ is —H;
R₃ is —H:
R₄ is chosen from —H or —CH₃, or R₁ and R₄ together form a cyclopropane, a cyclobutane, a cyclopentane, or an oxetane ring;
R₅ is chosen from —H, —CH₃, CF₃, —CH₂OH, —COOCH₃, —COOH, or —CH₂OCH₃;
R₆ is chosen from —H or —CH₃, or R₅ and R₆ together form an azetidine, pyrrolidine, morpholine or piperidine ring, each of which optionally comprises at least one substituent chosen from —CH₃, —OH, —CF₃, or —F;
R₇ is 1, 2, or 3 and independently chosen from —Cl, —F, —CF₃, —CH₃, —OCHF₂, or —OCH₃;
R₈ is chosen from benzene, —CH₃, or tertbutyl;
R₉ is chosen from a cyclohexane optionally comprising at least one —F or —CH₃ substituent and optionally substituted with —O—, or a benzimidazole;
R₁₀ is —C(CH₃)₃, cyclopentyl, —(CH₂) C(CH₃)₃, or a cyclohexane optionally substituted with —N— and optionally comprising at least one substituent chosen from —F or ═O, a cyclopentane optionally comprising an —OCH₃ or an —OH substituent, a tertbutyl, —CF₃, or a cyclopropyl optionally comprising a methyl substituent;
A₁ is chosen from —CH or —N;
A₂ is independently chosen from —CH, —N, or —O; and
A₃ is chosen from —O, CH₂, or CF₂;
X₂ is chosen from —CH₂CONH—, —CH₂—, —CH₂NHCO—, —CH₂NHCOCH₂—, —CH₂NHCO(CH₂)₂—, —NHCO—, —NHCH₂CONH—, —N(CH₃)CH₂CONH—, —CH₂N(CH₃)CO—; —CONH—, —CONHCH₂—, CONHCH₂C(CH₂CH₃)₂—, or —CH₂NH—; and
X₃ is a right-hand substitution of the diazaspiroheptane core chosen from:
an adamantane ring,
a benzofuran,
a phenyl group optionally comprising at least one substituent chosen independently from a halogen, —CH₃, —CF₃, —CHF₂, —OCHF₂, —CN, —OCH(CH₃)₂, —CH₂CH₃, —OCH₂CHF₂, —OCH₃, or cyclopentane,
a cyclohexane optionally comprising a 1,4-CH₂CH₂ bridge,
an imidazole or a benzimidazole, optionally comprising at least one substituent chosen independently from fluorine, chlorine, cyclohexane, —CH₃, —OCHF₂, benzene optionally comprising a halogen substituent, isobutyl, cyclopropyl, tertbutyl, methylpyrazole, —CH(CH₃)₂, or a methyl piperidine,
a pyridine optionally comprising at least one substituent chosen independently from methyl pyrazole, cyclopropyl, or —CH₃,
a naphthalene, or
an isoquinoline optionally comprising at least one halogen substituent,
or a pharmaceutically acceptable salt thereof.

2. A compound of Formula (I) having a diazaspiroheptane core: 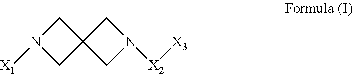
wherein X₁ is a left-hand substitution of the diazaspiroheptane core chosen from: 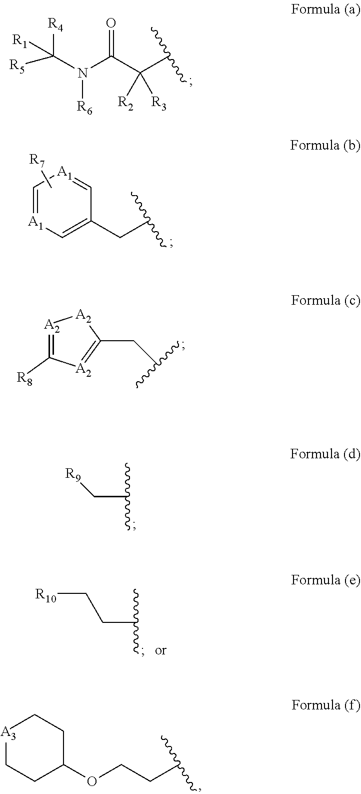
wherein R₁ is chosen from —H, —CH₃, —CH₂OCH₃, —CF₃, —CH₂CH₃, or —(CH₂)₂OCH₃;
R₂ is —H;
R₃ is —H;
R₄ is chosen from —H, —CH₃ or R₁ and R₄ together form a cyclobutane, a cyclopentane, or an oxetane ring;
R₅ is chosen from —H, —CH₃, —CH₂OH, —COOCH₃, —COOH, or —CH₂OCH₃;
R₆ is chosen from —H or —CH₃, or R₅ and R₆ together form an azetidine, pyrrolidine, morpholine or piperidine ring, each of which optionally comprises at least one substituent chosen from —CH₃, —OH, —CF₃, or —F;
R₇ is 1, 2, or 3, and independent chosen from —Cl, —F, —CF₃, —CHF₂, —CH₃, —OCHF₂, or —OCH₃;
R₅ is chosen from benzene, —CH₃, or tertbutyl;
R₉ is chosen from a cyclohexane optionally comprising at least one —F or —CH₃ substituent and optionally substituted with —O—, a benzimidazole, or —CO(CH₂)₂C(CH₃)₃;
R₁₀ is a cyclohexane optionally substituted with —N— and optionally comprising at least one substituent chosen from —F or ═O, a cyclopentane optionally comprising an —OCH₃ or an —OH substituent, a tertbutyl, or —CF₃;
A₁ is chosen from —CH or —N;
A₂ is independently chosen from —CH, —N, or —O; and
A₃ is chosen from —O, CH₂, or CF₂;
wherein X₂ is chosen from —CH₂CONH—, —CH₂—, —CH₂NHCO—, —CH₂NHCOCH₂—, —CH₂NHCO(CH₂)₂—, —NHCO—, —NHCH₂CONH—, —N(CH₃)CH₂CONH—, —CH₂N(CH₃)CO—; —CONH—, —CONHCH₂—, CONHCH₂C(CH₂CH₃)₂—, or —CH₂NH—; and
wherein X₃ is a right-hand substitution of the diazaspiroheptane core chosen from:
an adamantane ring,
a benzofuran,
a phenyl group optionally comprising at least one substituent chosen independently from a halogen, —CH₃, —CF₃, —CHF₂, —OCHF₂, —CN, —OCH(CH₃)₂, —CH₂CH₃, —OCH₂CHF₂, —OCH₃, or cyclopentane,
a cyclohexane optionally comprising a 1,4-CH₂CH₂ bridge,
an imidazole or a benzimidazole, optionally comprising at least one substituent chosen independently from chlorine, cyclohexane, —CH₃, benzene optionally comprising a halogen substituent, isobutyl, cyclopropyl, tertbutyl, methylpyrazole, —CH(CH₃)₂, or a methyl piperidine,
a pyridine optionally comprising at least one substituent chosen independently from methyl pyrazole, cyclopropyl, or —CH₃,
a naphthalene, or
an isoquinoline optionally comprising at least one halogen substituent,
or a pharmaceutically acceptable salt thereof.

3. The compound according to claim 1 or 2, wherein X₁ is Formula (a).

4. The compound according to claim 3, wherein in Formula (a), each of R₁, R₄, and R₅ is —CH₃.

5. The compound according to claim 3 or 4, wherein each of R₂, R₃, and R₆ is —H.

6. The compound according to any of claims 1-5, wherein X₂ is chosen from —CH₂CONH— or —CH₂—.

7. The compound according to any of claims 1-6, wherein X₂ is —CH₂CONH—.

8. The compound according to any of claims 1-7, wherein X₃ is an adamantane ring.

9. The compound according to any of claims 1-7, wherein X₃ is a phenyl group.

10. The compound according to claim 9, wherein the phenyl group comprises at least one halogen substituent.

11. The compound according to claim 10, wherein the at least one halogen is chosen from fluorine or chlorine.

12. The compound according to claim 11, wherein the at least one halogen comprises fluorine and chlorine.

13. The compound according to any of claims 1-12, wherein the compound comprises at least one deuterium.

14. The compound according to claim 13, wherein the at least one deuterium is in X₁.

15. The compound according to claim 2, wherein the compound is chosen from: 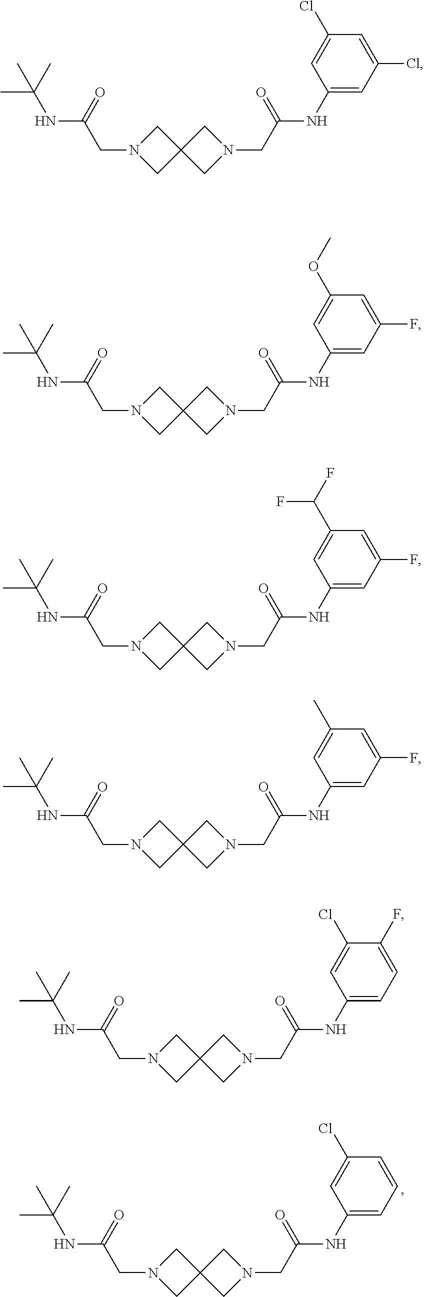 [SMILES: CC(C)(C)NC(=O)CN1CC2(CN(CC(=O)Nc3cc(Cl)cc(Cl)c3)C2)C1 ; COc1cc(F)cc(NC(=O)CN2CC3(C2)CN(CC(=O)NC(C)(C)C)C3)c1 ; CC(C)(C)NC(=O)CN1CC2(CN(CC(=O)Nc3cc(F)cc(C(F)F)c3)C2)C1 ; Cc1cc(F)cc(NC(=O)CN2CC3(C2)CN(CC(=O)NC(C)(C)C)C3)c1 ; CC(C)(C)NC(=O)CN1CC2(CN(CC(=O)Nc3ccc(F)c(Cl)c3)C2)C1 ; CC(C)(C)NC(=O)CN1CC2(CN(CC(=O)Nc3cccc(Cl)c3)C2)C1] 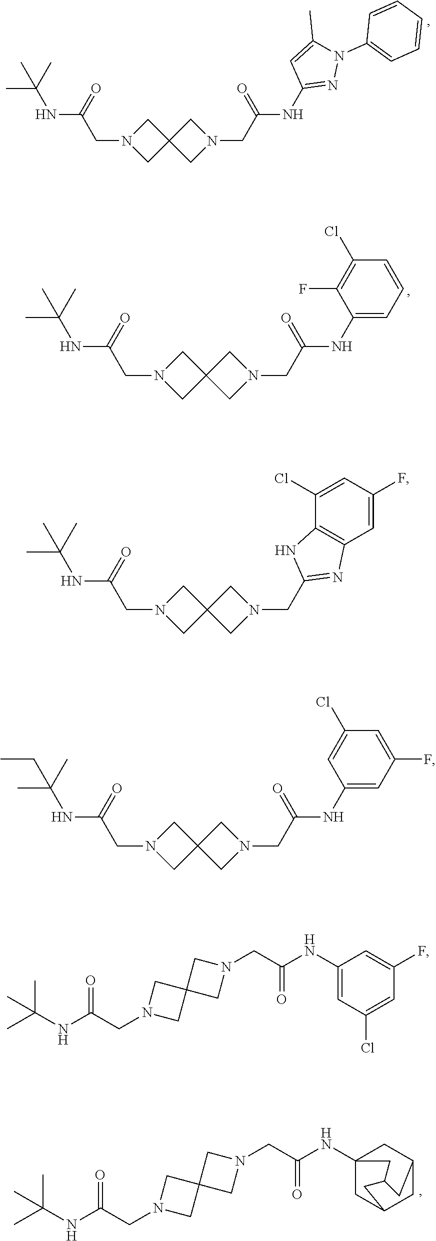 [SMILES: Cc1cc(NC(=O)CN2CC3(C2)CN(CC(=O)NC(C)(C)C)C3)nn1-c1ccccc1 ; CC(C)(C)NC(=O)CN1CC2(CN(CC(=O)Nc3cccc(Cl)c3F)C2)C1 ; CC(C)(C)NC(=O)CN1CC2(C1)CN(Cc1nc3cc(F)cc(Cl)c3[nH]1)C2 ; CCC(C)(C)NC(=O)CN1CC2(CN(CC(=O)Nc3cc(F)cc(Cl)c3)C2)C1 ; CC(C)(C)NC(=O)CN1CC2(CN(CC(=O)Nc3cc(F)cc(Cl)c3)C2)C1 ; CC(C)(C)NC(=O)CN1CC2(C1)CN(CC(=O)NC13CC4CC(CC(C4)C1)C3)C2] 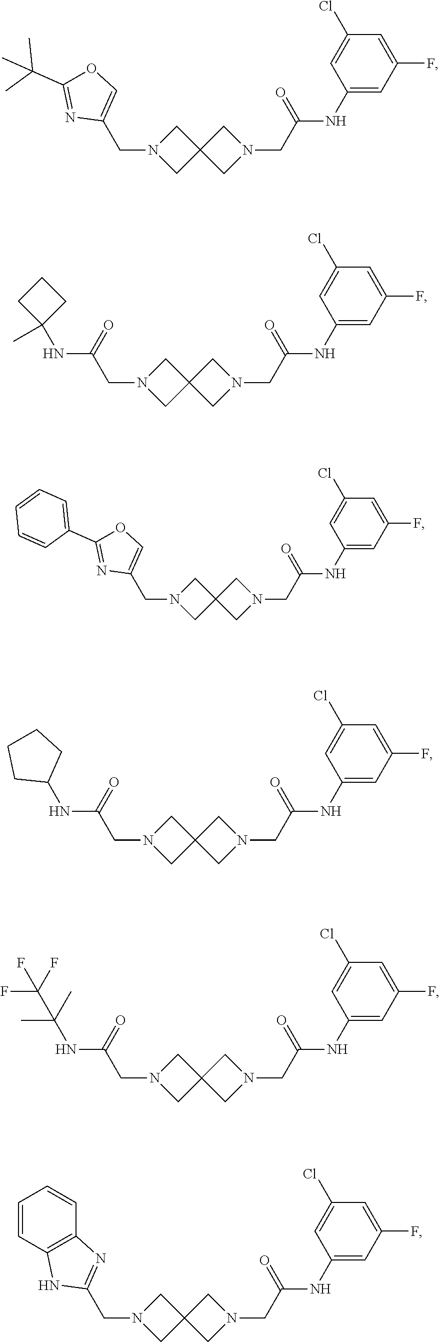 [SMILES: CC(C)(C)c1nc(CN2CC3(CN(CC(=O)Nc4cc(F)cc(Cl)c4)C3)C2)co1 ; CC1(NC(=O)CN2CC3(CN(CC(=O)Nc4cc(F)cc(Cl)c4)C3)C2)CCC1 ; O=C(CN1CC2(C1)CN(Cc1coc(-c3ccccc3)n1)C2)Nc1cc(F)cc(Cl)c1 ; O=C(CN1CC2(C1)CN(CC(=O)NC1CCCC1)C2)Nc1cc(F)cc(Cl)c1 ; CC(C)(NC(=O)CN1CC2(CN(CC(=O)Nc3cc(F)cc(Cl)c3)C2)C1)C(F)(F)F ; O=C(CN1CC2(C1)CN(Cc1nc3ccccc3[nH]1)C2)Nc1cc(F)cc(Cl)c1] 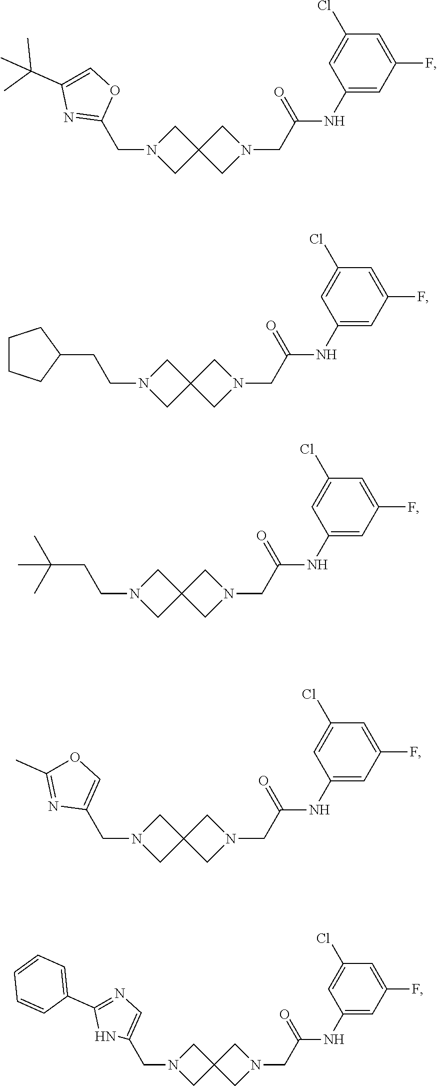 [SMILES: CC(C)(C)c1coc(CN2CC3(CN(CC(=O)Nc4cc(F)cc(Cl)c4)C3)C2)n1 ; O=C(CN1CC2(CN(CCC3CCCC3)C2)C1)Nc1cc(F)cc(Cl)c1 ; CC(C)(C)CCN1CC2(C1)CN(CC(=O)Nc1cc(F)cc(Cl)c1)C2 ; Cc1nc(CN2CC3(CN(CC(=O)Nc4cc(F)cc(Cl)c4)C3)C2)co1 ; O=C(CN1CC2(C1)CN(Cc1cnc(-c3ccccc3)[nH]1)C2)Nc1cc(F)cc(Cl)c1] 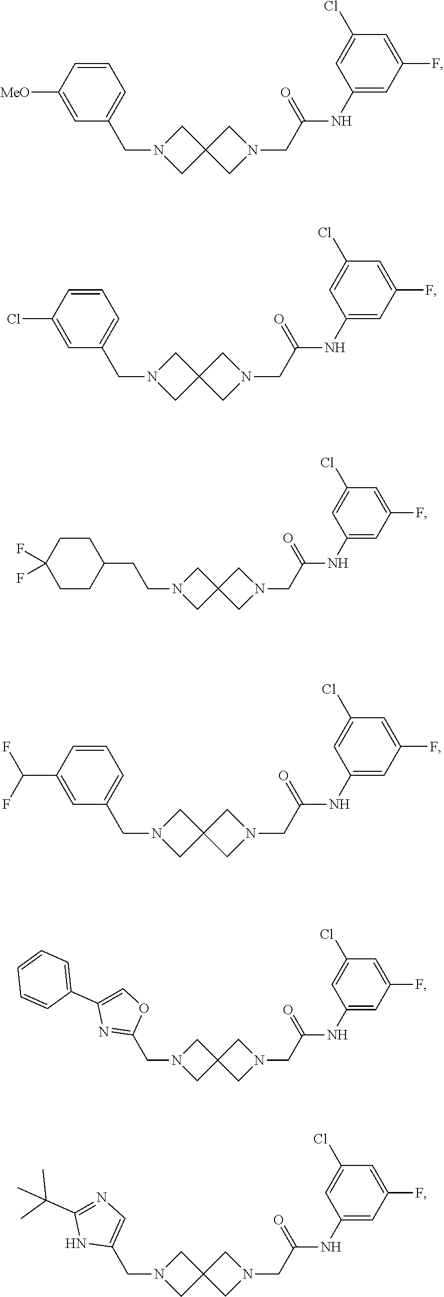 [SMILES: COc1cccc(CN2CC3(CN(CC(=O)Nc4cc(F)cc(Cl)c4)C3)C2)c1 ; O=C(CN1CC2(C1)CN(Cc1cccc(Cl)c1)C2)Nc1cc(F)cc(Cl)c1 ; O=C(CN1CC2(CN(CCC3CCC(F)(F)CC3)C2)C1)Nc1cc(F)cc(Cl)c1 ; O=C(CN1CC2(C1)CN(Cc1cccc(C(F)F)c1)C2)Nc1cc(F)cc(Cl)c1 ; O=C(CN1CC2(C1)CN(Cc1nc(-c3ccccc3)co1)C2)Nc1cc(F)cc(Cl)c1 ; CC(C)(C)c1ncc(CN2CC3(CN(CC(=O)Nc4cc(F)cc(Cl)c4)C3)C2)[nH]1] 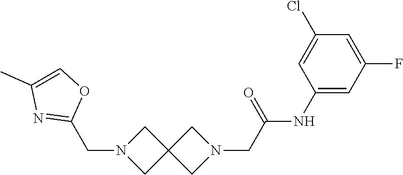 [SMILES: Cc1coc(CN2CC3(CN(CC(=O)Nc4cc(F)cc(Cl)c4)C3)C2)n1]
or a pharmaceutically acceptable salt thereof.

16. The compound according to claim 1, wherein the compound is chosen from: 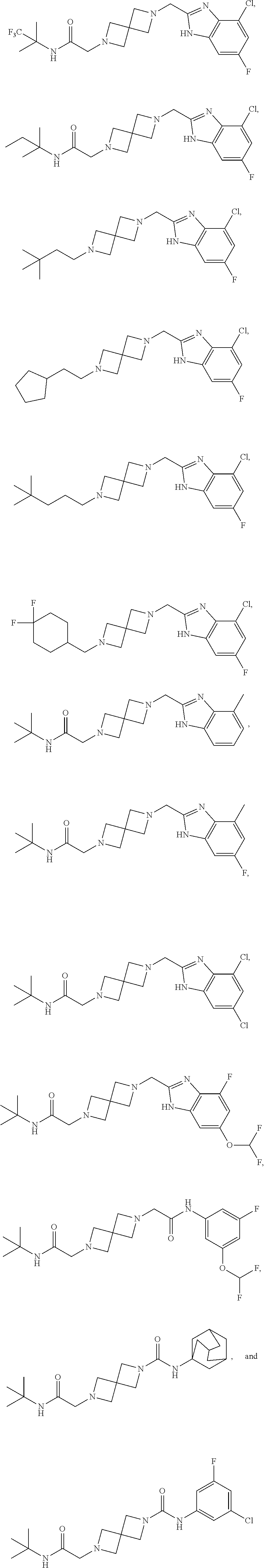
or a pharmaceutically acceptable salt thereof.

17. A pharmaceutical composition comprising the compound according to any of claims 1-16 and a pharmaceutically acceptable carrier.

18. The pharmaceutical composition according to claim 17, further comprising a modified-release polymer.

19. The pharmaceutical composition according to claim 18, wherein the modified-release polymer is hydroxypropyl methylcellulose, ethylcellulose, or a polyacrylate polymer.

20. A method of treating a disease or condition relating to aberrant function or activity of a T-type calcium channel in a subject in need thereof, the method comprising administering a therapeutically effective amount of the compound according to any of claims 1-16 or a therapeutically effective amount of the pharmaceutical composition according to any of claims 17-19 to the subject.

21. The method according to claim 20, wherein the disease or condition relating to aberrant function or activity of a T-type calcium channel is a psychiatric disorder, pain, tremor, seizures, epilepsy, or an epilepsy syndrome.

22. The method of claim 21, wherein the disease or condition relating to aberrant function or activity of a T-type calcium channel is tremor.

23. The method of claim 22, wherein the disease or condition relating to aberrant function or activity of a T-type calcium channel is essential tremor.
